SEQUENCE LISTING (578 sequences)

SEQ ID NO: 1 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 2 - protein, 515 aa, Homo sapiens [sequence omitted]

SEQ ID NO: 3 - protein, 513 aa, Mus sp.
MRSRCTVSAVGLLSLCLVVSASLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAIVGITTSCLVVMVFVSTIVMLKRIGYICLKDNLPNVLNFRHFLTWIIPERSPSEAIDRLEIIPTNKKKRLWNYDYEDGSDSDEEVPTASVTGYTMHGLTGKPLQQTSDTSASPEDPLHEEDSGAEESDEAGAGAGAEPELPTEAGAGPSEDPTGPYERRKSVLEDSFPREDNSSMDEPGDNIIFNVNLNSVFLRVLHDEDASETLSLEEDTILLDEGPQRTESDLRIAGGDRTQPPLPSLPSQDLWTEDGSSEKSDTSDSDADVGDGYIMR

SEQ ID NO: 4 - protein, 118 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSS

SEQ ID NO: 5 - protein, 107 aa, synthetic construct
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 6 - protein, 119 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 7 - protein, 107 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 8 - protein, 119 aa, synthetic construct
QVQLVQSGAEVKKPGASVKLSCKASGYNFKHTYAHWVRQAPGQGLEWIGRIDPANGNTKYDPKFQGRATMTADTASNTAYLELSSLRSEDTAVYYCVADHYGSSLLDYWGQGTLVTVSS

SEQ ID NO: 9 - protein, 112 aa, synthetic construct
DVVMTQTPLSLSVTPGQPASISCKSSQSLLDSDGKTYLNWFQQRPGQSPRRLIYLVSKLDSGVPDRFSGSGSGTDFTLKISRVEAEDVGVYYCWQGTYFPYTFGQGTKLEIK

SEQ ID NO: 10 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEYVRAEIMRSFSLSTNLQESLRSKE

SEQ ID NO: 11 - protein, 165 aa, synthetic construct
CDLPQTHSLGSRRTLMLLAQMRRISLFSCLKDDHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 12 - protein, 165 aa, synthetic construct
CDLPQTHSLGSRRTLMLLAQMRRISLFSCLKDRHDQGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 13 - protein, 165 aa, synthetic construct
CDLPQTHSLGSRRTLMLLAQMRRISLDSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 14 - protein, 165 aa, synthetic construct
CDLPQTHSLGSRRTLMLHAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 15 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRREIMRSFSLSTNLQESLRSKE

SEQ ID NO: 16 - protein, 165 aa, synthetic construct
CDLPQTHSLGSRRTLMLLAQMRRISLFSCLKDRHDEGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 17 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEEVRAEIMRSFSLSTNLQESLRSKE

SEQ ID NO: 18 - protein, 165 aa, synthetic construct
CDLPQTHSLGSRRTLMLLAQMRRISLFSCLKDGHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 19 - protein, 165 aa, synthetic construct
CDLPQTHSLGSRRTLMLLAQMRRISLFSCDKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 20 - protein, 165 aa, synthetic construct
CDLPQTHSLGSRRTLMLLAQMRRISLFSCEKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 21 - protein, 165 aa, synthetic construct
CDLPQTHSLGSRRTLMLLAQMRRISLFSCGKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 22 - protein, 165 aa, synthetic construct
CDLPQTHSLGSRRTLMLLAQMRRISLFSCAKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 23 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIRRSFSLSTNLQESLRSKE

SEQ ID NO: 24 - protein, 165 aa, synthetic construct
CDLPQTHSLGSRRTLMLLAQMRRISLFSCLKDKHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 25 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEHVRAEIMRSFSLSTNLQESLRSKE

SEQ ID NO: 26 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEKVRAEIMRSFSLSTNLQESLRSKE

SEQ ID NO: 27 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEISRSFSLSTNLQESLRSKE

SEQ ID NO: 28 - protein, 165 aa, synthetic construct
CDLPQTHSLGSRRTLMLDAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 29 - protein, 165 aa, synthetic construct
CDLPQTHSLGSRRTLMLLAQMRRISLFSCLKDRHDIGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 30 - protein, 165 aa, synthetic construct
CDLPQTHSLGSRRTLMLLAQMRRISLFSCLKDNHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 31 - protein, 165 aa, synthetic construct
CDLPQTHSLGSRRTLMLLAQMRRISLFSCLKDRPDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 32 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVWAEIMRSFSLSTNLQESLRSKE

SEQ ID NO: 33 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRHEIMRSFSLSTNLQESLRSKE

SEQ ID NO: 34 - protein, 165 aa, synthetic construct
CDLPQTHSLGSRRTLMLLAQMRRISLFSCLKDRHDNGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 35 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEITRSFSLSTNLQESLRSKE

SEQ ID NO: 36 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEWVRAEIMRSFSLSTNLQESLRSKE

SEQ ID NO: 37 - protein, 165 aa, synthetic construct
CDLPQTHSLGSRRTLMLLAQMRRISHFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 38 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEFVRAEIMRSFSLSTNLQESLRSKE

SEQ ID NO: 39 - protein, 165 aa, synthetic construct
CDLPQTHSLGSRRTLMLLAQMRRISLFSCNKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 40 - protein, 165 aa, synthetic construct
CDLPQTHSLGSRRTLMLLAQMRRISLFSCLKDRHDAGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 41 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRKEIMRSFSLSTNLQESLRSKE

SEQ ID NO: 42 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMDSFSLSTNLQESLRSKE

SEQ ID NO: 43 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVIAEIMRSFSLSTNLQESLRSKE

SEQ ID NO: 44 - protein, 165 aa, synthetic construct
CDLPQTHSLGSRRTLMLKAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 45 - protein, 165 aa, synthetic construct
CDLPQTHSLGSRRTLMLLAQMRRISLFSCLKDRHDGGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 46 - protein, 165 aa, synthetic construct
CDLPQTHSLGSRRTLMLLAQMRRISLFSCLKDVHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 47 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMWSFSLSTNLQESLRSKE

SEQ ID NO: 48 - protein, 165 aa, synthetic construct
CDLPQTHSLGSRRTLMLLAQMRRISLFSCLKDTHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 49 - protein, 165 aa, synthetic construct
CDLPQTHSLGSRRTLMLLAQMRRISIFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 50 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMNSFSLSTNLQESLRSKE

SEQ ID NO: 51 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEINRSFSLSTNLQESLRSKE

SEQ ID NO: 52 - protein, 165 aa, synthetic construct
CDLPQTHSLGSRRTLMLLAQMRRISKFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 53 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEQVRAEIMRSFSLSTNLQESLRSKE

SEQ ID NO: 54 - protein, 165 aa, synthetic construct
CDLPQTHSLGSRRTLMLLAQMRRISLFSCVKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 55 - protein, 165 aa, synthetic construct
CDLPQTHSLGSRRTLMLLAQMRRISLFSCSKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 56 - protein, 165 aa, synthetic construct
CDLPQTHSLGSRRTLMLGAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 57 - protein, 165 aa, synthetic construct
CDLPQTHSLGSRRTLMLLAQMRRISLFSCLKDAHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 58 - protein, 165 aa, synthetic construct
CDLPQTHSLGSRRTLMLLAQMRRISLFSCLKDRHDTGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 59 - protein, 165 aa, synthetic construct
CDLPQTHSLGSRRTLMLLAQMRRISLFSCLKDLHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 60 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRKFSLSTNLQESLRSKE

SEQ ID NO: 61 - protein, 165 aa, synthetic construct
CDLPQTHSLGSRRTLMLLAQMRRISLFSCLKDHHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 62 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVMAEIMRSFSLSTNLQESLRSKE

SEQ ID NO: 63 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 64 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVSAEIMRSFSLSTNLQESLRSKE

SEQ ID NO: 65 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMVSFSLSTNLQESLRSKE

SEQ ID NO: 66 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWELVRAEIMRSFSLSTNLQESLRSKE

SEQ ID NO: 67 - protein, 165 aa, synthetic construct
CDLPQTHSLGSRRTLMLLAQMRRPSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 68 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRRFSLSTNLQESLRSKE

SEQ ID NO: 69 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVEAEIMRSFSLSTNLQESLRSKE

SEQ ID NO: 70 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 71 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIYRSFSLSTNLQESLRSKE

SEQ ID NO: 72 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIWRSFSLSTNLQESLRSKE

SEQ ID NO: 73 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEMVRAEIMRSFSLSTNLQESLRSKE

SEQ ID NO: 74 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIFRSFSLSTNLQESLRSKE

SEQ ID NO: 75 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRYEIMRSFSLSTNLQESLRSKE

SEQ ID NO: 76 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWESVRAEIMRSFSLSTNLQESLRSKE

SEQ ID NO: 77 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWERVRAEIMRSFSLSTNLQESLRSKE

SEQ ID NO: 78 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFRLSTNLQESLRSKE

SEQ ID NO: 79 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 80 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMGSFSLSTNLQESLRSKE

SEQ ID NO: 81 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIKRSFSLSTNLQESLRSKE

SEQ ID NO: 82 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIERSFSLSTNLQESLRSKE

SEQ ID NO: 83 - protein, 165 aa, synthetic construct
CDLPQTHSLGSRRTLMLLAQMRPISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 84 - protein, 165 aa, synthetic construct
CDLPQTHSLGSRRTLMLLAQMRDISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 85 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMTSFSLSTNLQESLRSKE

SEQ ID NO: 86 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVLAEIMRSFSLSTNLQESLRSKE

SEQ ID NO: 87 - protein, 165 aa, synthetic construct
CDLPQTHSLGSRRTLMLLAQMRRISLFSCLKDRHDYGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 88 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSNSTNLQESLRSKE

SEQ ID NO: 89 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRATIMRSFSLSTNLQESLRSKE

SEQ ID NO: 90 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSGSTNLQESLRSKE

SEQ ID NO: 91 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVVAEIMRSFSLSTNLQESLRSKE

SEQ ID NO: 92 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMSSFSLSTNLQESLRSKE

SEQ ID NO: 93 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRHFSLSTNLQESLRSKE

SEQ ID NO: 94 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIGRSFSLSTNLQESLRSKE

SEQ ID NO: 95 - protein, 165 aa, synthetic construct
CDLPQTHSLGSRRTLMLLAQMRRISVFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 96 - protein, 165 aa, synthetic construct
CDLPQTHSLGSRRTLMLLAQMRRISLNSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 97 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVAAEIMRSFSLSTNLQESLRSKE

SEQ ID NO: 98 - protein, 165 aa, synthetic construct
CDLPQTHSLGSRRTLMLLAQMRRDSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 99 - protein, 165 aa, synthetic construct
CDLPQTHSLGSRRTLMLLAQMRFISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 100 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFKLSTNLQESLRSKE

SEQ ID NO: 101 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEILRSFSLSTNLQESLRSKE

SEQ ID NO: 102 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGWGVTETPLMKEDSILAVRKYFQRITLYLKEKKYSPCAWEVVRAEIMRSFSLSTNLQESLRSKE

SEQ ID NO: 103 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFMLSTNLQESLRSKE

SEQ ID NO: 104 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSHSTNLQESLRSKE

SEQ ID NO: 105 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRFEIMRSFSLSTNLQESLRSKE

SEQ ID NO: 106 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIIRSFSLSTNLQESLRSKE

SEQ ID NO: 107 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVGAEIMRSFSLSTNLQESLRSKE

SEQ ID NO: 108 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWENVRAEIMRSFSLSTNLQESLRSKE

SEQ ID NO: 109 - protein, 165 aa, synthetic construct
CDLPQTHSLGSRRTLMLLAQMRYISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 110 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIVRSFSLSTNLQESLRSKE

SEQ ID NO: 111 - protein, 165 aa, synthetic construct
CDLPQTHSLGSRRTHMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 112 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSASTNLQESLRSKE

SEQ ID NO: 113 - protein, 165 aa, synthetic construct
CDLPQTHSLGSRRTLMLAAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 114 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSPSTNLQESLRSKE

SEQ ID NO: 115 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSDSTNLQESLRSKE

SEQ ID NO: 116 - protein, 165 aa, synthetic construct
CDLPQTHSLGARRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 117 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWLRSKE

SEQ ID NO: 118 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLWDLEACVIQGVGVTETPLMKEDSILAVRKYFQRITLYLKEKKYSPCAWEVVRAEIMRSFSLSTNLQESLRSKE

SEQ ID NO: 119 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMREFSLSTNLQESLRSKE

SEQ ID NO: 120 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFLLSTNLQESLRSKE

SEQ ID NO: 121 - protein, 165 aa, synthetic construct
CDLPQTHSLGSRRTLMLLAQMRRISLGSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 122 - protein, 165 aa, synthetic construct
CDLPQTHSLGSRRTLMLLAQMRRISLFSCLKDRHDFGFPQEEFGNQFQKAETIPVLHEWIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 123 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSRSTNLQESLRSKE

SEQ ID NO: 124 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFPLSTNLQESLRSKE

SEQ ID NO: 125 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKI

SEQ ID NO: 126 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFILSTNLQESLRSKE

SEQ ID NO: 127 - protein, 165 aa, synthetic construct
CDLPQTHSLGSRRTLMLLAQMRRISLFSCLKTRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 128 - protein, 165 aa, synthetic construct
CDLPQTHSLGSRRTLMLLAQMRRISYFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 129 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMYSFSLSTNLQESLRSKE

SEQ ID NO: 130 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSYLAVRKYFQRITLYLKEKKYSPCAWEVVRAEIMRSFSLSTNLQESLRSKE

SEQ ID NO: 131 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSRE

SEQ ID NO: 132 - protein, 165 aa, synthetic construct
CDLPQTHSLGSRRTLMLLAQMRRISLFSCLKDRHDFGFPQEEFGNQFQKAETIPVLHEKIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 133 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLDTNLQESLRSKE

SEQ ID NO: 134 - protein, 165 aa, synthetic construct
CDLPQTHSLGSRRTLMLLAQMRRISLFSCLKDRHDFGFPQEEFGNQFQKAETIPVLHERIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 135 - protein, 165 aa, synthetic construct
CDLPQTHSLGSRRTLMLLAPMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 136 - protein, 165 aa, synthetic construct
CDLPQTHSLGSRRTLMLLAQMRRISLQSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 137 - protein, 165 aa, synthetic construct
CDLPQTHSLGSRRTLMLLAQMRNISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 138 - protein, 165 aa, synthetic construct
CDLPQTHSLGSRRTYMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 139 - protein, 165 aa, synthetic construct
CDLPQTHSLGSRRTLMLLAQMRRISLSSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 140 - protein, 165 aa, synthetic construct
CDLPQTHSLGSRRTGMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 141 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMLSFSLSTNLQESLRSKE

SEQ ID NO: 142 - protein, 165 aa, synthetic construct
CDLPQTHSLGSRRTLMLLAQMRRISLFSCLKDRHDFGFPEEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 143 - protein, 165 aa, synthetic construct
CDLPQTHSLGSRRTLMLLAQMRRISFFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 144 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQVVGVTETPLMKEDSILAVRKYFQRITLYLKEKKYSPCAWEVVRAEIMRSFSLSTNLQESLRSKE

SEQ ID NO: 145 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMMSFSLSTNLQESLRSKE

SEQ ID NO: 146 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSQSTNLQESLRSKE

SEQ ID NO: 147 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFVLSTNLQESLRSKE

SEQ ID NO: 148 - protein, 165 aa, synthetic construct
CDLPQTHSLGSRRTPMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 149 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSECAWEVVRAEIMRSFSLSTNLQESLRSKE

SEQ ID NO: 150 - protein, 165 aa, synthetic construct
CDLPQTHSLGSRRTLMLLAQMRSISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 151 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRNFSLSTNLQESLRSKE

SEQ ID NO: 152 - protein, 165 aa, synthetic construct
CDLPQTHSLGSRRTLMRLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 153 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQYITLYLKEKKYSPCAWEVVRAEIMRSFSLSTNLQESLRSKE

SEQ ID NO: 154 - protein, 165 aa, synthetic construct
CDLPQTHSLGSRRTLMLLAQMRRIYLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 155 - protein, 165 aa, synthetic construct
CDLPQTHSLGSRRTLMLLAQMRRISLMSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 156 - protein, 165 aa, synthetic construct
CDLPQTHSLGSRRTLMLLAQMRIISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 157 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDLFYTELYQQLNDLEACVIQGVGVTETPLMKEDSILAVRKYFQRITLYLKEKKYSPCAWEVVRAEIMRSFSLSTNLQESLRSKE

SEQ ID NO: 158 - protein, 165 aa, synthetic construct
CDLPQTHDLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 159 - protein, 165 aa, synthetic construct
CDLPQTHSLGSRRTLMLLAQMRRISLFSCLKDRHDFGGPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 160 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSFLAVRKYFQRITLYLKEKKYSPCAWEVVRAEIMRSFSLSTNLQESLRSKE

SEQ ID NO: 161 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLDSKE

SEQ ID NO: 162 - protein, 165 aa, synthetic construct
CDLPQTHSLGSRRTLMLLAQMRGISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 163 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFSRITLYLKEKKYSPCAWEVVRAEIMRSFSLSTNLQESLRSKE

SEQ ID NO: 164 - protein, 165 aa, synthetic construct
CDLPQTHSLGSRRTLMLLAQMPRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 165 - protein, 165 aa, synthetic construct
CDLPQTHSLGSRRTLMLLAQMRRISLFSCLKDRHDFGFPQEEFGNQFQKAETIPVLHEGIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 166 - protein, 165 aa, synthetic construct
CDLPQTHSLGSRRTLMLLAQMRRISGFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 167 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSESTNLQESLRSKE

SEQ ID NO: 168 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSLE

SEQ ID NO: 169 - protein, 165 aa, synthetic construct
CDLPQTHSLGSRRTLMGLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 170 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMKSFSLSTNLQESLRSKE

SEQ ID NO: 171 - protein, 165 aa, synthetic construct
CDLPQTHSLGSRRTLMLLAQMRRISLFSCLKDRHDFGFPQEEFGNQFQKAETIPELHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 172 - protein, 165 aa, synthetic construct
CDLPQTHSLGSRRTLMLLAQMRRISLFSCLKDRHDFGIPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 173 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSVSTNLQESLRSKE

SEQ ID NO: 174 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRASIMRSFSLSTNLQESLRSKE

SEQ ID NO: 175 - protein, 165 aa, synthetic construct
CDLPQTHSLGSRRTLMLLAQMRRISLFSCLLDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 176 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAGIMRSFSLSTNLQESLRSKE

SEQ ID NO: 177 - protein, 165 aa, synthetic construct
CDLPQTHSLGSRRTLMLLAQMRRISLFSCLKDRHDFGVPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 178 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRWFSLSTNLQESLRSKE

SEQ ID NO: 179 - protein, 165 aa, synthetic construct
CDLPQTHSLGSRRTLMLLAQMRRISLFSCLKDRHDFGPPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 180 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRLKE

SEQ ID NO: 181 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSEE

SEQ ID NO: 182 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSYE

SEQ ID NO: 183 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFWLSTNLQESLRSKE

SEQ ID NO: 184 - protein, 165 aa, synthetic construct
CDLPQTHSLGSRRTSMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 185 - protein, 165 aa, synthetic construct
CDLPQTHSLGSRRTLMLLAQMRRISEFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 186 - protein, 165 aa, synthetic construct
CDLPQTHSLGSRRTLMLLAQMRRISLFSCLKDRHDFGQPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 187 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYWQLNDLEACVIQGVGVTETPLMKEDSILAVRKYFQRITLYLKEKKYSPCAWEVVRAEIMRSFSLSTNLQESLRSKE

SEQ ID NO: 188 - protein, 165 aa, synthetic construct
CDLPQTHSLGSRRTLMLLAQMRRISLFSCLIDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 189 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEDKYSPCAWEVVRAEIMRSFSLSTNLQESLRSKE

SEQ ID NO: 190 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETWLMKEDSILAVRKYFQRITLYLKEKKYSPCAWEVVRAEIMRSFSLSTNLQESLRSKE

SEQ ID NO: 191 - protein, 165 aa, synthetic construct
CDLPQTHSLGSRRTLMLLAQMRRISLFDCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 192 - protein, 165 aa, synthetic construct
CDLPQTHSLGSRRTLMLLAQQRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 193 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLYACVIQGVGVTETPLMKEDSILAVRKYFQRITLYLKEKKYSPCAWEVVRAEIMRSFSLSTNLQESLRSKE

SEQ ID NO: 194 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRDEIMRSFSLSTNLQESLRSKE

SEQ ID NO: 195 - protein, 165 aa, synthetic construct
CDLPQTHSLGSRRTLMLLAQMRRISLHSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 196 - protein, 165 aa, synthetic construct
CDLPQTHSLGSRRTLMLLAQMRRISLFECLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 197 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEFLRSKE

SEQ ID NO: 198 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSFCAWEVVRAEIMRSFSLSTNLQESLRSKE

SEQ ID NO: 199 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTPLYQQLNDLEACVIQGVGVTETPLMKEDSILAVRKYFQRITLYLKEKKYSPCAWEVVRAEIMRSFSLSTNLQESLRSKE

SEQ ID NO: 200 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAELMRSFSLSTNLQESLRSKE

SEQ ID NO: 201 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFYLSTNLQESLRSKE

SEQ ID NO: 202 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFGLSTNLQESLRSKE

SEQ ID NO: 203 - protein, 165 aa, synthetic construct
CDLPQTHSLGSRRTLMQLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 204 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSVE

SEQ ID NO: 205 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKYYSPCAWEVVRAEIMRSFSLSTNLQESLRSKE

SEQ ID NO: 206 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLMDLEACVIQGVGVTETPLMKEDSILAVRKYFQRITLYLKEKKYSPCAWEVVRAEIMRSFSLSTNLQESLRSKE

SEQ ID NO: 207 - protein, 165 aa, synthetic construct
CDLPQTHSLGSRRTLMLLAQMRRISLFSCLKDRHDFGFPQEEFGNQFQKAETIPVLHEMIQQLFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 208 - protein, 165 aa, synthetic construct
CDLPQTHSLGSRRTLMLLAQMRRISLFSCLKDRHDFGFIQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 209 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKHFQRITLYLKEKKYSPCAWEVVRAEIMRSFSLSTNLQESLRSKE

SEQ ID NO: 210 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGYGVTETPLMKEDSILAVRKYFQRITLYLKEKKYSPCAWEVVRAEIMRSFSLSTNLQESLRSKE

SEQ ID NO: 211 - protein, 165 aa, synthetic construct
CDLPQTHSLGSRRTLMLLAQMRRISLFSCLKDRHDFGFPQEEFGNSFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 212 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSME

SEQ ID NO: 213 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLFDLEACVIQGVGVTETPLMKEDSILAVRKYFQRITLYLKEKKYSPCAWEVVRAEIMRSFSLSTNLQESLRSKE

SEQ ID NO: 214 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSWCAWEVVRAEIMRSFSLSTNLQESLRSKE

SEQ ID NO: 215 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSFE

SEQ ID NO: 216 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSIE

SEQ ID NO: 217 - protein, 165 aa, synthetic construct
CDLPQTHSLGSRRTTMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 218 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEMLRSKE

SEQ ID NO: 219 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEDVRAEIMRSFSLSTNLQESLRSKE

SEQ ID NO: 220 - protein, 165 aa, synthetic construct
CDLPQTHSLGSRRTLMLLGQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 221 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKELKYSPCAWEVVRAEIMRSFSLSTNLQESLRSKE

SEQ ID NO: 222 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEELRSKE

SEQ ID NO: 223 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFKTELYQQLNDLEACVIQGVGVTETPLMKEDSILAVRKYFQRITLYLKEKKYSPCAWEVVRAEIMRSFSLSTNLQESLRSKE

SEQ ID NO: 224 - protein, 165 aa, synthetic construct
CDLPQTHSLGSRRTLMLLAQMRRISLFSCLKNRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 225 - protein, 165 aa, synthetic construct
CDLPQTHSLGSRRTQMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 226 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLESKE

SEQ ID NO: 227 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRIKE

SEQ ID NO: 228 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFELSTNLQESLRSKE

SEQ ID NO: 229 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 230 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEYLRSKE

SEQ ID NO: 231 - protein, 165 aa, synthetic construct
CDLPQTHSLGSRRTLMLLAQMRAISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 232 - protein, 165 aa, synthetic construct
CDLPQTASLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 233 - protein, 165 aa, synthetic construct
CDLPQTHSLGSRRTLMLLAQMRHISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 234 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTSLYQQLNDLEACVIQGVGVTETPLMKEDSILAVRKYFQRITLYLKEKKYSPCAWEVVRAEIMRSFSLSTNLQESLRSKE

SEQ ID NO: 235 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEWKYSPCAWEVVRAEIMRSFSLSTNLQESLRSKE

SEQ ID NO: 236 - protein, 165 aa, synthetic construct
CDLPQTHSLGSRRTLMLLAQMRTISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 237 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFQLSTNLQESLRSKE

SEQ ID NO: 238 - protein, 165 aa, synthetic construct
CDLPQTHSLGSRRTLMLLAQMRRISLFSCLKDRHQFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 239 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKHKKYSPCAWEVVRAEIMRSFSLSTNLQESLRSKE

SEQ ID NO: 240 - protein, 165 aa, synthetic construct
CDLPQTHSLGSRRTLMDLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 241 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEMCVIQGVGVTETPLMKEDSILAVRKYFQRITLYLKEKKYSPCAWEVVRAEIMRSFSLSTNLQESLRSKE

SEQ ID NO: 242 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVHAEIMRSFSLSTNLQESLRSKE

SEQ ID NO: 243 - protein, 165 aa, synthetic construct
CDLPQTHSLGSRRTLMLLAQMRRISLFSCLKDRHDFGFPQEEFGNQFQKAETIPVLHESIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 244 - protein, 165 aa, synthetic construct
CDLPQTHSLGSRRTLMLLMQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 245 - protein, 165 aa, synthetic construct
CDLPQTHSLGSRRTLMLLAQMRRIHLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 246 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVFKYFQRITLYLKEKKYSPCAWEVVRAEIMRSFSLSTNLQESLRSKE

SEQ ID NO: 247 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSWE

SEQ ID NO: 248 - protein, 165 aa, synthetic construct
CDLPQTHSLGSRRTLMLLAQMRRSSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 249 - protein, 165 aa, synthetic construct
CDLPQTHSLGSNRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 250 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFTLSTNLQESLRSKE

SEQ ID NO: 251 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRYFSLSTNLQESLRSKE

SEQ ID NO: 252 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRMEIMRSFSLSTNLQESLRSKE

SEQ ID NO: 253 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAEWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 254 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRDKE

SEQ ID NO: 255 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLYKEDSILAVRKYFQRITLYLKEKKYSPCAWEVVRAEIMRSFSLSTNLQESLRSKE

SEQ ID NO: 256 - protein, 165 aa, synthetic construct
CDLPQTDSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 257 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLFACVIQGVGVTETPLMKEDSILAVRKYFQRITLYLKEKKYSPCAWEVVRAEIMRSFSLSTNLQESLRSKE

SEQ ID NO: 258 - protein, 165 aa, synthetic construct
CDLPQTHSLGSRRTLMLLAQMRRISLFSCLKDRHDFGFPQEEFGNQFQKAETIPGLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 259 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLQSKE

SEQ ID NO: 260 - protein, 165 aa, synthetic construct
CDLPQTHSLGSRRTLMLLAQMRRISLFSCLMDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 261 - protein, 165 aa, synthetic construct
CDLPQTHSDGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 262 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETELDKFYTELYQQLNDLEACVIQGVGVTETPLMKEDSILAVRKYFQRITLYLKEKKYSPCAWEVVRAEIMRSFSLSTNLQESLRSKE

SEQ ID NO: 263 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDDFYTELYQQLNDLEACVIQGVGVTETPLMKEDSILAVRKYFQRITLYLKEKKYSPCAWEVVRAEIMRSFSLSTNLQESLRSKE

SEQ ID NO: 264 - protein, 165 aa, synthetic construct
CDLPQTHSLGSRRTNMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 265 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTGLQESLRSKE

SEQ ID NO: 266 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEIVRAEIMRSFSLSTNLQESLRSKE

SEQ ID NO: 267 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETDLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 268 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSGE

SEQ ID NO: 269 - protein, 165 aa, synthetic construct
CDLPQTHSLGSRGTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 270 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILEVRKYFQRITLYLKEKKYSPCAWEVVRAEIMRSFSLSTNLQESLRSKE

SEQ ID NO: 271 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVGETPLMKEDSILAVRKYFQRITLYLKEKKYSPCAWEVVRAEIMRSFSLSTNLQESLRSKE

SEQ ID NO: 272 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRQEIMRSFSLSTNLQESLRSKE

SEQ ID NO: 273 - protein, 165 aa, synthetic construct
CDLPQTHSLGSRRTLMLLAQMRRISLFSCLKQRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 274 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEKLRSKE

SEQ ID NO: 275 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQELLRSKE

SEQ ID NO: 276 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLQDLEACVIQGVGVTETPLMKEDSILAVRKYFQRITLYLKEKKYSPCAWEVVRAEIMRSFSLSTNLQESLRSKE

SEQ ID NO: 277 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLRTNLQESLRSKE

SEQ ID NO: 278 - protein, 165 aa, synthetic construct
CDLPQTHSLGSRRTLMLLAQMRRISLFSCLKDRHDFGFPQEEFGNQFQKAETIPVLHEMIQQIFNLFSTEDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 279 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILIVRKYFQRITLYLKEKKYSPCAWEVVRAEIMRSFSLSTNLQESLRSKE

SEQ ID NO: 280 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYWELYQQLNDLEACVIQGVGVTETPLMKEDSILAVRKYFQRITLYLKEKKYSPCAWEVVRAEIMRSFSLSTNLQESLRSKE

SEQ ID NO: 281 - protein, 165 aa, synthetic construct
CDLPQTHSLGSRRTLMLLAQMRRISLFSCLKDRHDFGFPQEEFGNQFQDAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 282 - protein, 165 aa, synthetic construct
CDLPQTHSLGSRRTLMLLAQMRRISLASCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 283 - protein, 165 aa, synthetic construct
CDLPQTHSLGSRRTLMLLAQMRRISLFSCLKDRHDFGAPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 284 - protein, 165 aa, synthetic construct
CDLPQTHSLGSRRTLMLLAQMRRISLFSCLKDRHDFGSPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 285 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSYCAWEVVRAEIMRSFSLSTNLQESLRSKE

SEQ ID NO: 286 - protein, 165 aa, synthetic construct
CDLPQTHSEGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 287 - protein, 165 aa, synthetic construct
CDLPQTHSLGSRRTLMLLAQMRRISLFSCLKDRHDFGFPQEEFGNQFQKAETIPVLHEMIQQIFNLFSTVDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 288 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSTCAWEVVRAEIMRSFSLSTNLQESLRSKE

SEQ ID NO: 289 - protein, 165 aa, synthetic construct
CDLPQTHSLGSRRTLMLLAQMRRESLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 290 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRREIMRSFSLSTNLQESLRSKE

SEQ ID NO: 291 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFPLSTNLQESLRSKE

SEQ ID NO: 292 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRHFSLSTNLQESLRSKE

SEQ ID NO: 293 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIWRSFSLSTNLQESLRSKE

SEQ ID NO: 294 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIGRSFSLSTNLQESLRSKE

SEQ ID NO: 295 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRRFSLSTNLQESLRSKE

SEQ ID NO: 296 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFPLSTNLQESLRSKE

SEQ ID NO: 297 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIVRSFSLSTNLQESLRSKE

SEQ ID NO: 298 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIKRSFSLSTNLQESLRSKE

SEQ ID NO: 299 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEISRSFSLSTNLQESLRSKE

SEQ ID NO: 300 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFRLSTNLQESLRSKE

SEQ ID NO: 301 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIRRSFSLSTNLQESLRSKE

SEQ ID NO: 302 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSGSTNLQESLRSKE

SEQ ID NO: 303 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIYRSFSLSTNLQESLRSKE

SEQ ID NO: 304 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSNSTNLQESLRSKE

SEQ ID NO: 305 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRQFSLSTNLQESLRSKE

SEQ ID NO: 306 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSHSTNLQESLRSKE

SEQ ID NO: 307 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSRSTNLQESLRSKE

SEQ ID NO: 308 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 309 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEITRSFSLSTNLQESLRSKE

SEQ ID NO: 310 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSASTNLQESLRSKE

SEQ ID NO: 311 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIFRSFSLSTNLQESLRSKE

SEQ ID NO: 312 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFKLSTNLQESLRSKE

SEQ ID NO: 313 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFKLSTNLQESLRSKE

SEQ ID NO: 314 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSESTNLQESLRSKE

SEQ ID NO: 315 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRATIMRSFSLSTNLQESLRSKE

SEQ ID NO: 316 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEILRSFSLSTNLQESLRSKE

SEQ ID NO: 317 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSQSTNLQESLRSKE

SEQ ID NO: 318 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSEE

SEQ ID NO: 319 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRNFSLSTNLQESLRSKE

SEQ ID NO: 320 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSHSTNLQESLRSKE

SEQ ID NO: 321 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFRLSTNLQESLRSKE

SEQ ID NO: 322 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLNTNLQESLRSKE

SEQ ID NO: 323 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRWFSLSTNLQESLRSKE

SEQ ID NO: 324 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLDTNLQESLRSKE

SEQ ID NO: 325 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLDSKE

SEQ ID NO: 326 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRASIMRSFSLSTNLQESLRSKE

SEQ ID NO: 327 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFGLSTNLQESLRSKE

SEQ ID NO: 328 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLLTNLQESLRSKE

SEQ ID NO: 329 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMKSFSLSTNLQESLRSKE

SEQ ID NO: 330 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLESKE

SEQ ID NO: 331 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSME

SEQ ID NO: 332 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSWE

SEQ ID NO: 333 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYQPCAWEVVRAEIMRSFSLSTNLQESLRSKE

SEQ ID NO: 334 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSQSTNLQESLRSKE

SEQ ID NO: 335 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLESKE

SEQ ID NO: 336 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFQLSTNLQESLRSKE

SEQ ID NO: 337 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFMLSTNLQESLRSKE

SEQ ID NO: 338 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKVKKYSPCAWEVVRAEIMRSFSLSTNLQESLRSKE

SEQ ID NO: 339 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSDE

SEQ ID NO: 340 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFWLSTNLQESLRSKE

SEQ ID NO: 341 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSVSTNLQESLRSKE

SEQ ID NO: 342 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRLFSLSTNLQESLRSKE

SEQ ID NO: 343 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFGLSTNLQESLRSKE

SEQ ID NO: 344 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLGSKE

SEQ ID NO: 345 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVWKYFQRITLYLKEKKYSPCAWEVVRAEIMRSFSLSTNLQESLRSKE

SEQ ID NO: 346 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRTTLYLKEKKYSPCAWEVVRAEIMRSFSLSTNLQESLRSKE

SEQ ID NO: 347 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLLSKE

SEQ ID NO: 348 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLSSKE

SEQ ID NO: 349 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLHTNLQESLRSKE

SEQ ID NO: 350 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDDFYTELYQQLNDLEACVIQGVGVTETPLMKEDSILAVRKYFQRITLYLKEKKYSPCAWEVVRAEIMRSFSLSTNLQESLRSKE

SEQ ID NO: 351 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLNSKE

SEQ ID NO: 352 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSVE

SEQ ID NO: 353 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDWFYTELYQQLNDLEACVIQGVGVTETPLMKEDSILAVRKYFQRITLYLKEKKYSPCAWEVVRAEIMRSFSLSTNLQESLRSKE

SEQ ID NO: 354 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMTEDSILAVRKYFQRITLYLKEKKYSPCAWEVVRAEIMRSFSLSTNLQESLRSKE

SEQ ID NO: 355 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGITETPLMKEDSILAVRKYFQRITLYLKEKKYSPCAWEVVRAEIMRSFSLSTNLQESLRSKE

SEQ ID NO: 356 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLDSKE

SEQ ID NO: 357 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDPFYTELYQQLNDLEACVIQGVGVTETPLMKEDSILAVRKYFQRITLYLKEKKYSPCAWEVVRAEIMRSFSLSTNLQESLRSKE

SEQ ID NO: 358 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEMLRSKE

SEQ ID NO: 359 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEDLRSKE

SEQ ID NO: 360 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEAKYSPCAWEVVRAEIMRSFSLSTNLQESLRSKE

SEQ ID NO: 361 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQELLRSKE

SEQ ID NO: 362 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSDE

SEQ ID NO: 363 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLMSKE

SEQ ID NO: 364 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLNSKE

SEQ ID NO: 365 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRLEIMRSFSLSTNLQESLRSKE

SEQ ID NO: 366 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFLLSTNLQESLRSKE

SEQ ID NO: 367 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLQSKE

SEQ ID NO: 368 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSGE

SEQ ID NO: 369 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLETNLQESLRSKE

SEQ ID NO: 370 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCALEVVRAEIMRSFSLSTNLQESLRSKE

SEQ ID NO: 371 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLWSKE

SEQ ID NO: 372 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSTE

SEQ ID NO: 373 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYWELYQQLNDLEACVIQGVGVTETPLMKEDSILAVRKYFQRITLYLKEKKYSPCAWEVVRAEIMRSFSLSTNLQESLRSKE

SEQ ID NO: 374 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSECAWEVVRAEIMRSFSLSTNLQESLRSKE

SEQ ID NO: 375 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDTFYTELYQQLNDLEACVIQGVGVTETPLMKEDSILAVRKYFQRITLYLKEKKYSPCAWEVVRAEIMRSFSLSTNLQESLRSKE

SEQ ID NO: 376 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLTSKE

SEQ ID NO: 377 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFILSTNLQESLRSKE

SEQ ID NO: 378 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRFFSLSTNLQESLRSKE

SEQ ID NO: 379 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLLSKE

SEQ ID NO: 380 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEELRSKE

SEQ ID NO: 381 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSNE

SEQ ID NO: 382 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSFE

SEQ ID NO: 383 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYDPCAWEVVRAEIMRSFSLSTNLQESLRSKE

SEQ ID NO: 384 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDIFYTELYQQLNDLEACVIQGVGVTETPLMKEDSILAVRKYFQRITLYLKEKKYSPCAWEVVRAEIMRSFSLSTNLQESLRSKE

SEQ ID NO: 385 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFYLSTNLQESLRSKE

SEQ ID NO: 386 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESNRSKE

SEQ ID NO: 387 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAELMRSFSLSTNLQESLRSKE

SEQ ID NO: 388 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVSRAEIMRSFSLSTNLQESLRSKE

SEQ ID NO: 389 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSAE

SEQ ID NO: 390 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRDKE

SEQ ID NO: 391 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTEFYQQLNDLEACVIQGVGVTETPLMKEDSILAVRKYFQRITLYLKEKKYSPCAWEVVRAEIMRSFSLSTNLQESLRSKE

SEQ ID NO: 392 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLQEKKYSPCAWEVVRAEIMRSFSLSTNLQESLRSKE

SEQ ID NO: 393 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDTFYTELYQQLNDLEACVIQGVGVTETPLMKEDSILAVRKYFQRITLYLKEKKYSPCAWEVVRAEIMRSFSLSTNLQESLRSKE

SEQ ID NO: 394 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKDDSILAVRKYFQRITLYLKEKKYSPCAWEVVRAEIMRSFSLSTNLQESLRSKE

SEQ ID NO: 395 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDMFYTELYQQLNDLEACVIQGVGVTETPLMKEDSILAVRKYFQRITLYLKEKKYSPCAWEVVRAEIMRSFSLSTNLQESLRSKE

SEQ ID NO: 396 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKWYTELYQQLNDLEACVIQGVGVTETPLMKEDSILAVRKYFQRITLYLKEKKYSPCAWEVVRAEIMRSFSLSTNLQESLRSKE

SEQ ID NO: 397 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLYSKE

SEQ ID NO: 398 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEIVRAEIMRSFSLSTNLQESLRSKE

SEQ ID NO: 399 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSWE

SEQ ID NO: 400 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLISKE

SEQ ID NO: 401 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLTSKE

SEQ ID NO: 402 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLVSKE

SEQ ID NO: 403 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYAQLNDLEACVIQGVGVTETPLMKEDSILAVRKYFQRITLYLKEKKYSPCAWEVVRAEIMRSFSLSTNLQESLRSKE

SEQ ID NO: 404 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSKNLQESLRSKE

SEQ ID NO: 405 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEVKYSPCAWEVVRAEIMRSFSLSTNLQESLRSKE

SEQ ID NO: 406 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSSE

SEQ ID NO: 407 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQILNDLEACVIQGVGVTETPLMKEDSILAVRKYFQRITLYLKEKKYSPCAWEVVRAEIMRSFSLSTNLQESLRSKE

SEQ ID NO: 408 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTEEPLMKEDSILAVRKYFQRITLYLKEKKYSPCAWEVVRAEIMRSFSLSTNLQESLRSKE

SEQ ID NO: 409 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEELRSKE

SEQ ID NO: 410 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLWLKEKKYSPCAWEVVRAEIMRSFSLSTNLQESLRSKE

SEQ ID NO: 411 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGETETPLMKEDSILAVRKYFQRITLYLKEKKYSPCAWEVVRAEIMRSFSLSTNLQESLRSKE

SEQ ID NO: 412 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKKKKYSPCAWEVVRAEIMRSFSLSTNLQESLRSKE

SEQ ID NO: 413 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFTLSTNLQESLRSKE

SEQ ID NO: 414 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKRKKYSPCAWEVVRAEIMRSFSLSTNLQESLRSKE

SEQ ID NO: 415 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVTRAEIMRSFSLSTNLQESLRSKE

SEQ ID NO: 416 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSFNLQESLRSKE

SEQ ID NO: 417 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRWEIMRSFSLSTNLQESLRSKE

SEQ ID NO: 418 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVTAEIMRSFSLSTNLQESLRSKE

SEQ ID NO: 419 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVLAEIMRSFSLSTNLQESLRSKE

SEQ ID NO: 420 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVVAEIMRSFSLSTNLQESLRSKE

SEQ ID NO: 421 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRYEIMRSFSLSTNLQESLRSKE

SEQ ID NO: 422 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMIEDSILAVRKYFQRITLYLKEKKYSPCAWEVVRAEIMRSFSLSTNLQESLRSKE

SEQ ID NO: 423 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTMTPLMKEDSILAVRKYFQRITLYLKEKKYSPCAWEVVRAEIMRSFSLSTNLQESLRSKE

SEQ ID NO: 424 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVDVTETPLMKEDSILAVRKYFQRITLYLKEKKYSPCAWEVVRAEIMRSFSLSTNLQESLRSKE

SEQ ID NO: 425 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVIAEIMRSFSLSTNLQESLRSKE

SEQ ID NO: 426 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLETCVIQGVGVTETPLMKEDSILAVRKYFQRITLYLKEKKYSPCAWEVVRAEIMRSFSLSTNLQESLRSKE

SEQ ID NO: 427 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRDEIMRSFSLSTNLQESLRSKE

SEQ ID NO: 428 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKR

SEQ ID NO: 429 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGEGVTETPLMKEDSILAVRKYFQRITLYLKEKKYSPCAWEVVRAEIMRSFSLSTNLQESLRSKE

SEQ ID NO: 430 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDLFYTELYQQLNDLEACVIQGVGVTETPLMKEDSILAVRKYFQRITLYLKEKKYSPCAWEVVRAEIMRSFSLSTNLQESLRSKE

SEQ ID NO: 431 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSINAVRKYFQRITLYLKEKKYSPCAWEVVRAEIMRSFSLSTNLQESLRSKE

SEQ ID NO: 432 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGGTETPLMKEDSILAVRKYFQRITLYLKEKKYSPCAWEVVRAEIMRSFSLSTNLQESLRSKE

SEQ ID NO: 433 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNEQESLRSKE

SEQ ID NO: 434 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRFEIMRSFSLSTNLQESLRSKE

SEQ ID NO: 435 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRWEIMRSFSLSTNLQESLRSKE

SEQ ID NO: 436 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFDLSTNLQESLRSKE

SEQ ID NO: 437 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEQKYSPCAWEVVRAEIMRSFSLSTNLQESLRSKE

SEQ ID NO: 438 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRHEIMRSFSLSTNLQESLRSKE

SEQ ID NO: 439 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMNSFSLSTNLQESLRSKE

SEQ ID NO: 440 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVSAEIMRSFSLSTNLQESLRSKE

SEQ ID NO: 441 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVGAEIMRSFSLSTNLQESLRSKE

SEQ ID NO: 442 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMESFSLSTNLQESLRSKE

SEQ ID NO: 443 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMGSFSLSTNLQESLRSKE

SEQ ID NO: 444 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYDPCAWEVVRAEIMRSFSLSTNLQESLRSKE

SEQ ID NO: 445 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEGVRAEIMRSFSLSTNLQESLRSKE

SEQ ID NO: 446 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVREEIMRSFSLSTNLQESLRSKE

SEQ ID NO: 447 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEKVRAEIMRSFSLSTNLQESLRSKE

SEQ ID NO: 448 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSRE

SEQ ID NO: 449 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVIKYFQRITLYLKEKKYSPCAWEVVRAEIMRSFSLSTNLQESLRSKE

SEQ ID NO: 450 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEDVRAEIMRSFSLSTNLQESLRSKE

SEQ ID NO: 451 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEEKYSPCAWEVVRAEIMRSFSLSTNLQESLRSKE

SEQ ID NO: 452 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRKEIMRSFSLSTNLQESLRSKE

SEQ ID NO: 453 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEFVRAEIMRSFSLSTNLQESLRSKE

SEQ ID NO: 454 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMDSFSLSTNLQESLRSKE

SEQ ID NO: 455 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRQEIMRSFSLSTNLQESLRSKE

SEQ ID NO: 456 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEQVRAEIMRSFSLSTNLQESLRSKE

SEQ ID NO: 457 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMNSFSLSTNLQESLRSKE

SEQ ID NO: 458 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRDEIMRSFSLSTNLQESLRSKE

SEQ ID NO: 459 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEQKYSPCAWEVVRAEIMRSFSLSTNLQESLRSKE

SEQ ID NO: 460 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWESVRAEIMRSFSLSTNLQESLRSKE

SEQ ID NO: 461 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRKKE

SEQ ID NO: 462 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEYVRAEIMRSFSLSTNLQESLRSKE

SEQ ID NO: 463 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEEVRAEIMRSFSLSTNLQESLRSKE

SEQ ID NO: 464 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKKKKYSPCAWEVVRAEIMRSFSLSTNLQESLRSKE

SEQ ID NO: 465 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLDTNLQESLRSKE

SEQ ID NO: 466 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIERSFSLSTNLQESLRSKE

SEQ ID NO: 467 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMDSFSLSTNLQESLRSKE

SEQ ID NO: 468 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEMVRAEIMRSFSLSTNLQESLRSKE

SEQ ID NO: 469 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMWSFSLSTNLQESLRSKE

SEQ ID NO: 470 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWERVRAEIMRSFSLSTNLQESLRSKE

SEQ ID NO: 471 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMWSFSLSTNLQESLRSKE

SEQ ID NO: 472 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMQSFSLSTNLQESLRSKE

SEQ ID NO: 473 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKRKKYSPCAWEVVRAEIMRSFSLSTNLQESLRSKE

SEQ ID NO: 474 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVTAEIMRSFSLSTNLQESLRSKE

SEQ ID NO: 475 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 476 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVWAEIMRSFSLSTNLQESLRSKE

SEQ ID NO: 477 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMTSFSLSTNLQESLRSKE

SEQ ID NO: 478 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMSSFSLSTNLQESLRSKE

SEQ ID NO: 479 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWETVRAEIMRSFSLSTNLQESLRSKE

SEQ ID NO: 480 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQYVGVTETPLMKEDSILAVRKYFQRITLYLKEKKYSPCAWEVVRAEIMRSFSLSTNLQESLRSKE

SEQ ID NO: 481 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVAAEIMRSFSLSTNLQESLRSKE

SEQ ID NO: 482 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVIAEIMRSFSLSTNLQESLRSKE

SEQ ID NO: 483 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVFAEIMRSFSLSTNLQESLRSKE

SEQ ID NO: 484 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNGQESLRSKE

SEQ ID NO: 485 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWENVRAEIMRSFSLSTNLQESLRSKE

SEQ ID NO: 486 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVMAEIMRSFSLSTNLQESLRSKE

SEQ ID NO: 487 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVLAEIMRSFSLSTNLQESLRSKE

SEQ ID NO: 488 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMGSFSLSTNLQESLRSKE

SEQ ID NO: 489 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVAAEIMRSFSLSTNLQESLRSKE

SEQ ID NO: 490 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRRFSLSTNLQESLRSKE

SEQ ID NO: 491 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQKLNDLEACVIQGVGVTETPLMKEDSILAVRKYFQRITLYLKEKKYSPCAWEVVRAEIMRSFSLSTNLQESLRSKE

SEQ ID NO: 492 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRNEIMRSFSLSTNLQESLRSKE

SEQ ID NO: 493 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEHVRAEIMRSFSLSTNLQESLRSKE

SEQ ID NO: 494 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDWILAVRKYFQRITLYLKEKKYSPCAWEVVRAEIMRSFSLSTNLQESLRSKE

SEQ ID NO: 495 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESERSKE

SEQ ID NO: 496 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRHEIMRSFSLSTNLQESLRSKE

SEQ ID NO: 497 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMIEDSILAVRKYFQRITLYLKEKKYSPCAWEVVRAEIMRSFSLSTNLQESLRSKE

SEQ ID NO: 498 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRILLYLKEKKYSPCAWEVVRAEIMRSFSLSTNLQESLRSKE

SEQ ID NO: 499 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMVSFSLSTNLQESLRSKE

SEQ ID NO: 500 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSFNLQESLRSKE

SEQ ID NO: 501 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRSEIMRSFSLSTNLQESLRSKE

SEQ ID NO: 502 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSRSTNLQESLRSKE

SEQ ID NO: 503 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRIGLYLKEKKYSPCAWEVVRAEIMRSFSLSTNLQESLRSKE

SEQ ID NO: 504 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWLRSKE

SEQ ID NO: 505 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSESTNLQESLRSKE

SEQ ID NO: 506 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFELSTNLQESLRSKE

SEQ ID NO: 507 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVHAEIMRSFSLSTNLQESLRSKE

SEQ ID NO: 508 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRKEIMRSFSLSTNLQESLRSKE

SEQ ID NO: 509 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFDLSTNLQESLRSKE

SEQ ID NO: 510 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMGEDSILAVRKYFQRITLYLKEKKYSPCAWEVVRAEIMRSFSLSTNLQESLRSKE

SEQ ID NO: 511 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSRE

SEQ ID NO: 512 - protein, 165 aa, synthetic construct
CDLPQTHSLGSRRTLMLLAQNRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSEE

SEQ ID NO: 513 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRQEIMRSFSLSTNLQESLRSKE

SEQ ID NO: 514 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMISFSLSTNLQESLRSKE

SEQ ID NO: 515 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRMEIMRSFSLSTNLQESLRSKE

SEQ ID NO: 516 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMFSFSLSTNLQESLRSKE

SEQ ID NO: 517 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMMSFSLSTNLQESLRSKE

SEQ ID NO: 518 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSDSTNLQESLRSKE

SEQ ID NO: 519 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMYSFSLSTNLQESLRSKE

SEQ ID NO: 520 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIERSFSLSTNLQESLRSKE

SEQ ID NO: 521 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEDKYSPCAWEVVRAEIMRSFSLSTNLQESLRSKE

SEQ ID NO: 522 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 523 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRREIMRSFSLSTNLQESLRSKE

SEQ ID NO: 524 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMLSFSLSTNLQESLRSKE

SEQ ID NO: 525 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEFLRSKE

SEQ ID NO: 526 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSIE

SEQ ID NO: 527 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIDRSFSLSTNLQESLRSKE

SEQ ID NO: 528 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSYE

SEQ ID NO: 529 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSLE

SEQ ID NO: 530 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTGLQESLRSKE

SEQ ID NO: 531 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEEKYSPCAWEVVRAEIMRSFSLSTNLQESLRSKE

SEQ ID NO: 532 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVKAEIMRSFSLSTNLQESLRSKE

SEQ ID NO: 533 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEYLRSKE

SEQ ID NO: 534 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWDVVRAEIMRSFSLSTNLQESLRSKE

SEQ ID NO: 535 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSPSTNLQESLRSKE

SEQ ID NO: 536 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSFE

SEQ ID NO: 537 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKETKYSPCAWEVVRAEIMRSFSLSTNLQESLRSKE

SEQ ID NO: 538 - protein, 165 aa, synthetic construct
CDLPQTHSLGSRRTLMLLAQMRRISPFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYHPCAWEVVRAEIMRSFSLSTNLQESLRSKE

SEQ ID NO: 539 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRPSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 540 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 541 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMTSFSLSTNLQESLRSKE

SEQ ID NO: 542 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRFISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 543 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFKLSTNLQESLRSKE

SEQ ID NO: 544 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEILRSFSLSTNLQESLRSKE

SEQ ID NO: 545 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWLRSKE

SEQ ID NO: 546 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLWDLEACVIQGVGVTETPLMKEDSILAVRKYFQRITLYLKEKKYSPCAWEVVRAEIMRSFSLSTNLQESLRSKE

SEQ ID NO: 547 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLGSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 548 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEGIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 549 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGGPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 550 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLDSKE

SEQ ID NO: 551 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 552 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRPSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 553 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMTSFSLSTNLQESLRSKE

SEQ ID NO: 554 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFKLSTNLQESLRSKE

SEQ ID NO: 555 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEILRSFSLSTNLQESLRSKE

SEQ ID NO: 556 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWLRSKE

SEQ ID NO: 557 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLGSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 558 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGGPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 559 - protein, 647 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSSGGGGSGGGGSGGGGSDVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLDSKE

SEQ ID NO: 560 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRREIMRSFSLSTNLQESLRSKE

SEQ ID NO: 561 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMDSFSLSTNLQESLRSKE

SEQ ID NO: 562 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMNSFSLSTNLQESLRSKE

SEQ ID NO: 563 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDLHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 564 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVEAEIMRSFSLSTNLQESLRSKE

SEQ ID NO: 565 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIYRSFSLSTNLQESLRSKE

SEQ ID NO: 566 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRDISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 567 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRHFSLSTNLQESLRSKE

SEQ ID NO: 568 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRDSLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 569 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRFEIMRSFSLSTNLQESLRSKE

SEQ ID NO: 570 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWENVRAEIMRSFSLSTNLQESLRSKE

SEQ ID NO: 571 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGARRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 572 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSRE

SEQ ID NO: 573 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLSSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 574 - protein, 409 aa, synthetic construct
DIEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSECAWEVVRAEIMRSFSLSTNLQESLRSKE

SEQ ID NO: 575 - protein, 10 aa, synthetic construct
GGGGSGGGGS

SEQ ID NO: 576 - protein, 15 aa, synthetic construct
GGGGSGGGGSGGGGS

SEQ ID NO: 577 - protein, 6 aa, synthetic construct
HHHHHH

SEQ ID NO: 578 - protein, 8 aa, synthetic construct
HHHHHHHH

(19) United States
(12) Patent Application Publication
(10) Pub. No.: US 2024/0309062 A1
(43) Pub. Date: Sep. 19, 2024

(54) INTERFERON ALPHA-2 VARIANTS

(71) Applicant: A-Alpha Bio, Seattle, WA (US)

(72) Inventors: Emily Engelhart, Seattle, WA (US); Davis Goodnight, Seattle, WA (US); Kyle Minch, Seattle, WA (US); Ryan Swanson, Seattle, WA (US)

(21) Appl. No.: 18/607,194

(22) Filed: Mar. 15, 2024

Related U.S. Application Data
(60) Provisional application No. 63/497,089, filed on Apr. 19, 2023.
(60) Provisional application No. 63/452,654, filed on Mar. 16, 2023.

(51) Int. Cl. C07K 14/56; C07K 16/28

(52) U.S. Cl. CPC C07K 14/56; C07K 16/2815; C07K 2319/30

(57) ABSTRACT

Provided herein are human interferon alpha-2 (IFNA2) variants that have decreased or no detectable binding to the human interferon-alpha/beta receptor beta 2 (IFNAR2) as compared to the wild-type human IFNA2 polypeptide. Also provided herein are fusion proteins comprising an antibody or fragment thereof, and a human IFNA2 variant, wherein the IFNA2 variant is covalently linked to the antibody, and wherein the IFNA2 variant has decreased or no detectable binding to the human interferon-alpha/beta receptor beta (IFNAR2) as compared to the wild-type human IFNA2 polypeptide.

### CROSS-REFERENCE TO RELATED APPLICATIONS

This application claims the benefit of U.S. Provisional Patent Application No. 63/452,654, filed on Mar. 16, 2023, and U.S. Provisional Patent Application No. 63/497,089, filed on Apr. 19, 2023, the contents of which are incorporated herein by reference in their entirety.

### TECHNICAL FIELD

The present disclosure provides human interferon alpha-2 (IFNA2) variants, methods for making thereof, and methods for measuring binding affinities between IFNA2 variants and human interferon alpha/beta receptor 2 (IFNAR2). The provided IFNA2 variants and fusion proteins comprising IFNA2 variants can have therapeutic use. Also provided are methods of stimulating an immune response or suppressing cellular/tumor proliferation in a mammal, and methods of treating a disorder (e.g., cancer) using the IFNA2 variants or the fusion proteins of such IFNA2 variants.

### BACKGROUND

Cytokines, including IFNA2, are potent immune modulators with potential therapeutic value in immuno-oncology and infectious disease. However, in human subjects their potency can present challenges, including only modest efficacies accompanied by significant toxicities and adverse side effects due to immune activation in healthy tissues. Therefore, there exists a need for therapeutic agents based on a targeted cytokine, which would be of great clinical value in treatments of various diseases including cancer.

### SUMMARY

The present disclosure provides human interferon alpha-2 (IFNA2) variants and fusion proteins including those variants. In a therapeutic context, these cytokine variants can be targeted to a particular immune cell type of interest by linking the cytokine to an antibody or portion thereof against a specific cell surface marker. To ensure immune activation occurs only at the cell type of interest, the disclosure provides “detuned” cytokine variants, e.g., IFNA2 variants, with weakened affinity for their receptors (in this case, IFNA2 variants with weakened affinity for interferon alpha/beta receptor 2 IFNAR2). Detuning the cytokine can decrease or eliminate toxicities and adverse side effects by localizing the cytokine activity to a specific cellular or tumor context. By reducing affinity for the receptor, the activity of the detuned cytokines is decreased for most cell types, e.g., in healthy tissues, which, in turn, decreases potential toxicity of the molecule when administered as a therapy. However, at the surface of the cell type targeted by the antibody, the residual activity of the cytokine is sufficient to bind its receptor and activate pro-inflammatory pathways leading to immune activation.

The compositions and methods provided herein are based, at least in part, on the identification of IFNA2 variants with decreased affinity for the IFNAR2 receptor by high-throughput screening using a protein-protein interaction (PPI) assay based on a yeast sexual agglutination method, termed AlphaSeq™ (see, e.g., U.S. Pat. Nos. 10,988,759 and 11,136,573).

Cytokine signaling can trigger multifaceted and even opposing activities in different cell types, often leading to toxicity or poor response rates in the therapeutic setting. Targeting specific cellular signaling, such as defined immune subsets or antigen experienced cells at the tumor, has the potential to widen a cytokine therapeutic index and improve patient outcomes.

In a first aspect, the disclosure provides isolated human interferon alpha-2 (IFNA2) variants, wherein the IFNA2 variants have decreased or no detectable binding to the human interferon-alpha/beta receptor beta 2 (IFNAR2) as compared to the wild-type human IFNA2 polypeptide. In some embodiments, the isolated IFNA2 variant has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

In some embodiments, the isolated IFNA2 variants include one or more amino acid substitutions at one or more positions of the wild-type human IFNA2 polypeptide (SEQ ID NO: 1) selected from the group consisting of: H30, S31, L32, S34, R35, R36, L38, L40, L41, A42, Q43, M44, R45, R46, I47, S48, L49, F50, S51, L53, K54, D55, R56, H57, D58, F59, F61, P62, Q63, Q69, K72, V78, M82, Q84, I86, K93, A98, L103, K106, Y108, T109, E110, Q113, N116, N116, E119, A120, G125, V126, T129, P132, M134, I139, A141, R143, Y145, Q147, R148, E155, K156, K157, P160, V165, R167, A168, E169, I170, M171, R172, S173, S175, L176, S177, N179, S183, R185, S186, K187, and E188.

In some embodiments, the IFNA2 variants include an H30X amino acid substitution in SEQ ID NO: 1, where X represents any amino acid. In some embodiments, the isolated IFNA2 variant includes an H30A or H30D amino acid substitution in SEQ ID NO: 1.

In some embodiments, the IFNA2 variants include an S31X amino acid substitution in SEQ ID NO: 1, where X represents any amino acid. In some embodiments, the IFNA2 variant includes an S31D amino acid substitution in SEQ ID NO: 1.

In some embodiments, the IFNA2 variants include an L32X amino acid substitution in SEQ ID NO: 1, where X represents any amino acid. In some embodiments, the IFNA2 variant includes an L32D or L32E amino acid substitution in SEQ ID NO: 1.

In some embodiments, the IFNA2 variants include an S34X amino acid substitution in SEQ ID NO: 1, where X represents any amino acid. In some embodiments, the IFNA2 variant includes an S34A amino acid substitution in SEQ ID NO: 1.

In some embodiments, the IFNA2 variants include an R35X amino acid substitution in SEQ ID NO: 1, where X represents any amino acid. In some embodiments, the IFNA2 variant includes an R35N amino acid substitution in SEQ ID NO: 1.

In some embodiments, the IFNA2 variants include an R36X amino acid substitution in SEQ ID NO: 1, where X represents any amino acid. In some embodiments, the IFNA2 variant includes an R36G amino acid substitution in SEQ ID NO: 1.

In some embodiments, the IFNA2 variants include an L38X amino acid substitution in SEQ ID NO: 1, where X represents any amino acid. In some embodiments, the IFNA2 variant includes an L38H, L38Y, L38G, L38P, L38S, L38T, L38Q, or L38N amino acid substitution in SEQ ID NO: 1.

In some embodiments, the IFNA2 variants include an L40X amino acid substitution in SEQ ID NO: 1, where X represents any amino acid. In some embodiments, the IFNA2 variant includes an L40R, L40G, L40Q, or L40D amino acid substitution in SEQ ID NO: 1.

In some embodiments, the IFNA2 variants include an L41X amino acid substitution in SEQ ID NO: 1, where X represents any amino acid. In some embodiments, the IFNA2 variant includes an L41H, L41D, L41K, L41G, or L41A amino acid substitution in SEQ ID NO: 1.

In some embodiments, the IFNA2 variants include an A42X amino acid substitution in SEQ ID NO: 1, where X represents any amino acid. In some embodiments, the IFNA2 variant includes an A42G or A42M amino acid substitution in SEQ ID NO: 1.

In some embodiments, the IFNA2 variants include an Q43X amino acid substitution in SEQ ID NO: 1, where X represents any amino acid. In some embodiments, the IFNA2 variant includes an Q43P amino acid substitution in SEQ ID NO: 1.

In some embodiments, the IFNA2 variants include an M44X amino acid substitution in SEQ ID NO: 1, where X represents any amino acid. In some embodiments, the IFNA2 variant includes an M44Q amino acid substitution in SEQ ID NO: 1.

In some embodiments, the IFNA2 variants include an R45X amino acid substitution in SEQ ID NO: 1, where X represents any amino acid. In some embodiments, the IFNA2 variant includes an R45P amino acid substitution in SEQ ID NO: 1.

In some embodiments, the IFNA2 variants include an R46X amino acid substitution in SEQ ID NO: 1, where X represents any amino acid. In some embodiments, the IFNA2 variant includes an R46P, R46D, R46F, R46Y, R46N, R46S, R461, R46G, R46A, R46H, or R46T amino acid substitution in SEQ ID NO: 1.

In some embodiments, the IFNA2 variants include an 147X amino acid substitution in SEQ ID NO: 1, where X represents any amino acid. In some embodiments, the IFNA2 variant includes an 147P, 147D, 147S, or 147E amino acid substitution in SEQ ID NO: 1.

In some embodiments, the IFNA2 variants include an S48X amino acid substitution in SEQ ID NO: 1, where X represents any amino acid. In some embodiments, the IFNA2 variant includes an S48Y or S48H amino acid substitution in SEQ ID NO: 1.

In some embodiments, the IFNA2 variants include an L49X amino acid substitution in SEQ ID NO: 1, where X represents any amino acid. In some embodiments, the IFNA2 variant includes an L49H, L491, L49K, L49V, L49Y, L49F, L49G, L49E, or L49P amino acid substitution in SEQ ID NO: 1.

In some embodiments, the IFNA2 variants include an F50X amino acid substitution in SEQ ID NO: 1, where X represents any amino acid. In some embodiments, the IFNA2 variant includes an F50D, F50N, F50G, F50Q, F50S, F50M, F50H, or F50A amino acid substitution in SEQ ID NO: 1.

In some embodiments, the IFNA2 variants include an S51X amino acid substitution in SEQ ID NO: 1, where X represents any amino acid. In some embodiments, the IFNA2 variant includes an S51D or S51E amino acid substitution in SEQ ID NO: 1.

In some embodiments, the IFNA2 variants include an L53X amino acid substitution in SEQ ID NO: 1, where X represents any amino acid. In some embodiments, the IFNA2 variant includes an L53D, L53E, L53G, L53A, L53N, L53V, or L53S amino acid substitution in SEQ ID NO: 1.

In some embodiments, the IFNA2 variants include an K54X amino acid substitution in SEQ ID NO: 1, where X represents any amino acid. In some embodiments, the IFNA2 variant includes an K54L, K54I, or K54M amino acid substitution in SEQ ID NO: 1.

In some embodiments, the IFNA2 variants include an D55X amino acid substitution in SEQ ID NO: 1, where X represents any amino acid. In some embodiments, the IFNA2 variant includes an D55T, D55N, or D55Q amino acid substitution in SEQ ID NO: 1.

In some embodiments, the IFNA2 variants include an R56X amino acid substitution in SEQ ID NO: 1, where X represents any amino acid. In some embodiments, the IFNA2 variant includes an R56D, R56G, R56K, R56N, R56V, R56T, R56A, R56L, R56H amino acid substitution in SEQ ID NO: 1.

In some embodiments, the IFNA2 variants include an H57X amino acid substitution in SEQ ID NO: 1, where X represents any amino acid. In some embodiments, the IFNA2 variant includes an H57P amino acid substitution in SEQ ID NO: 1.

In some embodiments, the IFNA2 variants include an D58X amino acid substitution in SEQ ID NO: 1, where X represents any amino acid. In some embodiments, the IFNA2 variant includes an D58Q amino acid substitution in SEQ ID NO: 1.

In some embodiments, the IFNA2 variants include an F59X amino acid substitution in SEQ ID NO: 1, where X represents any amino acid. In some embodiments, the IFNA2 variant includes an F59Q, F59E, F591, F59N, F59A, F59G, F59T, or F59Y amino acid substitution in SEQ ID NO: 1.

In some embodiments, the IFNA2 variants include an F61X amino acid substitution in SEQ ID NO: 1, where X represents any amino acid. In some embodiments, the IFNA2 variant includes an F61G, F61I, F61V, F61P, F61Q, F61A, or F61S amino acid substitution in SEQ ID NO: 1.

In some embodiments, the IFNA2 variants include an P62X amino acid substitution in SEQ ID NO: 1, where X represents any amino acid. In some embodiments, the IFNA2 variant includes an P62I amino acid substitution in SEQ ID NO: 1.

In some embodiments, the IFNA2 variants include an Q63X amino acid substitution in SEQ ID NO: 1, where X represents any amino acid. In some embodiments, the IFNA2 variant includes an Q63E amino acid substitution in SEQ ID NO: 1.

In some embodiments, the IFNA2 variants include an Q69X amino acid substitution in SEQ ID NO: 1, where X represents any amino acid. In some embodiments, the IFNA2 variant includes an Q69S amino acid substitution in SEQ ID NO: 1.

In some embodiments, the IFNA2 variants include an K72X amino acid substitution in SEQ ID NO: 1, where X represents any amino acid. In some embodiments, the IFNA2 variant includes an K72D amino acid substitution in SEQ ID NO: 1.

In some embodiments, the IFNA2 variants include an V78X amino acid substitution in SEQ ID NO: 1, where X represents any amino acid. In some embodiments, the IFNA2 variant includes an V78E or V78G amino acid substitution in SEQ ID NO: 1.

In some embodiments, the IFNA2 variants include an M82X amino acid substitution in SEQ ID NO: 1, where X represents any amino acid. In some embodiments, the IFNA2 variant includes an M82W, M82K, M82R, M82G, or M82S amino acid substitution in SEQ ID NO: 1.

In some embodiments, the IFNA2 variants include an 186X amino acid substitution in SEQ ID NO: 1, where X represents any amino acid. In some embodiments, the IFNA2 variant includes an 186L amino acid substitution in SEQ ID NO: 1.

In some embodiments, the IFNA2 variants include an K93X amino acid substitution in SEQ ID NO: 1, where X represents any amino acid. In some embodiments, the IFNA2 variant includes an K93E or K93V amino acid substitution in SEQ ID NO: 1.

In some embodiments, the IFNA2 variants include an A98X amino acid substitution in SEQ ID NO: 1, where X represents any amino acid. In some embodiments, the IFNA2 variant includes an A98E amino acid substitution in SEQ ID NO: 1.

In some embodiments, the IFNA2 variants include an L103X amino acid substitution in SEQ ID NO: 1, where X represents any amino acid. In some embodiments, the IFNA2 variant includes an L103E or L103D amino acid substitution in SEQ ID NO: 1.

In some embodiments, the IFNA2 variants include an K106X amino acid substitution in SEQ ID NO: 1, where X represents any amino acid. In some embodiments, the IFNA2 variant includes an K106L or K106D amino acid substitution in SEQ ID NO: 1.

In some embodiments, the IFNA2 variants include an Y108X amino acid substitution in SEQ ID NO: 1, where X represents any amino acid. In some embodiments, the IFNA2 variant includes an Y108K amino acid substitution in SEQ ID NO: 1.

In some embodiments, the IFNA2 variants include an T109X amino acid substitution in SEQ ID NO: 1, where X represents any amino acid. In some embodiments, the IFNA2 variant includes an T109W amino acid substitution in SEQ ID NO: 1.

In some embodiments, the IFNA2 variants include an E110X amino acid substitution in SEQ ID NO: 1, where X represents any amino acid. In some embodiments, the IFNA2 variant includes an E110P or E110S amino acid substitution in SEQ ID NO: 1.

In some embodiments, the IFNA2 variants include an Q113X amino acid substitution in SEQ ID NO: 1, where X represents any amino acid. In some embodiments, the IFNA2 variant includes an Q113W amino acid substitution in SEQ ID NO: 1.

In some embodiments, the IFNA2 variants include an N116X amino acid substitution in SEQ ID NO: 1, where X represents any amino acid. In some embodiments, the IFNA2 variant includes an N116W, N116M, N116F, or N116Q amino acid substitution in SEQ ID NO: 1.

In some embodiments, the IFNA2 variants include an E119X amino acid substitution in SEQ ID NO: 1, where X represents any amino acid. In some embodiments, the IFNA2 variant includes an E119Y or E119F amino acid substitution in SEQ ID NO: 1.

In some embodiments, the IFNA2 variants include an A120X amino acid substitution in SEQ ID NO: 1, where X represents any amino acid. In some embodiments, the IFNA2 variant includes an A120M amino acid substitution in SEQ ID NO: 1.

In some embodiments, the IFNA2 variants include an G125X amino acid substitution in SEQ ID NO: 1, where X represents any amino acid. In some embodiments, the IFNA2 variant includes an G125V amino acid substitution in SEQ ID NO: 1.

In some embodiments, the IFNA2 variants include an V126X amino acid substitution in SEQ ID NO: 1, where X represents any amino acid. In some embodiments, the IFNA2 variant includes an V126W or V126Y amino acid substitution in SEQ ID NO: 1.

In some embodiments, the IFNA2 variants include an T129X amino acid substitution in SEQ ID NO: 1, where X represents any amino acid. In some embodiments, the IFNA2 variant includes an T129G amino acid substitution in SEQ ID NO: 1.

In some embodiments, the IFNA2 variants include an P132X amino acid substitution in SEQ ID NO: 1, where X represents any amino acid. In some embodiments, the IFNA2 variant includes an P132W amino acid substitution in SEQ ID NO: 1.

In some embodiments, the IFNA2 variants include an M134X amino acid substitution in SEQ ID NO: 1, where X represents any amino acid. In some embodiments, the IFNA2 variant includes an M134Y amino acid substitution in SEQ ID NO: 1.

In some embodiments, the IFNA2 variants include an M139X amino acid substitution in SEQ ID NO: 1, where X represents any amino acid. In some embodiments, the IFNA2 variant includes an I139Y or I139F amino acid substitution in SEQ ID NO: 1.

In some embodiments, the IFNA2 variants include an A141X amino acid substitution in SEQ ID NO: 1, where X represents any amino acid. In some embodiments, the IFNA2 variant includes an A141E or A141I amino acid substitution in SEQ ID NO: 1.

In some embodiments, the IFNA2 variants include an R143X amino acid substitution in SEQ ID NO: 1, where X represents any amino acid. In some embodiments, the IFNA2 variant includes an R143F amino acid substitution in SEQ ID NO: 1.

In some embodiments, the IFNA2 variants include an Q147X amino acid substitution in SEQ ID NO: 1, where X represents any amino acid. In some embodiments, the IFNA2 variant includes an Q147S amino acid substitution in SEQ ID NO: 1.

In some embodiments, the IFNA2 variants include an R148X amino acid substitution in SEQ ID NO: 1, where X represents any amino acid. In some embodiments, the IFNA2 variant includes an R148Y amino acid substitution in SEQ ID NO: 1.

In some embodiments, the IFNA2 variants include an E155X amino acid substitution in SEQ ID NO: 1, where X represents any amino acid. In some embodiments, the IFNA2 variant includes an E155H amino acid substitution in SEQ ID NO: 1.

In some embodiments, the IFNA2 variants include an K156X amino acid substitution in SEQ ID NO: 1, where X represents any amino acid. In some embodiments, the IFNA2 variant includes an K156D, K156L, or K156W amino acid substitution in SEQ ID NO: 1.

In some embodiments, the IFNA2 variants include an K157X amino acid substitution in SEQ ID NO: 1, where X represents any amino acid. In some embodiments, the IFNA2 variant includes an K157Y amino acid substitution in SEQ ID NO: 1.

In some embodiments, the IFNA2 variants include an P160X amino acid substitution in SEQ ID NO: 1, where X represents any amino acid. In some embodiments, the IFNA2 variant includes an P160E, P160F, P160W, P160Y, or P160T amino acid substitution in SEQ ID NO: 1.

In some embodiments, the IFNA2 variants include an V165X amino acid substitution in SEQ ID NO: 1, where X represents any amino acid. In some embodiments, the IFNA2 variant includes an V165Y, V165E, V165H, V165K, V165W, V165F, V165Q, V165L, V165M, V165S, V165R, V165N, V165D, or V165I amino acid substitution in SEQ ID NO: 1.

In some embodiments, the IFNA2 variants include an R167X amino acid substitution in SEQ ID NO: 1, where X represents any amino acid. In some embodiments, the IFNA2 variant includes an R167W, R167I, R167M, R167S, R167E, R167L, R167V, R167A, R167G, or R167H amino acid substitution in SEQ ID NO: 1.

In some embodiments, the IFNA2 variants include an A168X amino acid substitution in SEQ ID NO: 1, where X represents any amino acid. In some embodiments, the IFNA2 variant includes an A168R, A168H, A168K, A168Y, A168G, A168F, A168D, A168M, or A168Q amino acid substitution in SEQ ID NO: 1.

In some embodiments, the IFNA2 variants include an E169X amino acid substitution in SEQ ID NO: 1, where X represents any amino acid. In some embodiments, the IFNA2 variant includes an E169T, E169S, or E169G amino acid substitution in SEQ ID NO: 1.

In some embodiments, the IFNA2 variants include an 1170X amino acid substitution in SEQ ID NO: 1, where X represents any amino acid. In some embodiments, the IFNA2 variant includes an 1170L amino acid substitution in SEQ ID NO: 1.

In some embodiments, the IFNA2 variants include an M171X amino acid substitution in SEQ ID NO: 1, where X represents any amino acid. In some embodiments, the IFNA2 variant includes an M171R, M171S, M171T, M171N, M171A, M171Y, M171W, M171F, M171K, M171E, M171G, M171L, M171I, or M171V amino acid substitution in SEQ ID NO: 1.

In some embodiments, the IFNA2 variants include an R172X amino acid substitution in SEQ ID NO: 1, where X represents any amino acid. In some embodiments, the IFNA2 variant includes an R172D, R172W, R172N, R172A, R172V, R172G, R172T, R172S, R172Y, R172L, R172M, or R172K amino acid substitution in SEQ ID NO: 1.

In some embodiments, the IFNA2 variants include an S173X amino acid substitution in SEQ ID NO: 1, where X represents any amino acid. In some embodiments, the IFNA2 variant includes an S173K, S173R, S173H, S173E, S173N, S173W, or S173Y amino acid substitution in SEQ ID NO: 1.

In some embodiments, the IFNA2 variants includes an S175X amino acid substitution in SEQ ID NO: 1, where X represents any amino acid. In some embodiments, the IFNA2 variant includes an S175R, S175K, S175M, S175L, S175P, S175I, S175V, S175W, S175Y, S175G, S175E, S175Q, or S175T amino acid substitution in SEQ ID NO: 1.

In some embodiments, the IFNA2 variants include an L176X amino acid substitution in SEQ ID NO: 1, where X represents any amino acid. In some embodiments, the IFNA2 variant includes an L176N, L176G, L176H, L176A, L176P, L176D, L176R, L176Q, L176E, or L176V amino acid substitution in SEQ ID NO: 1.

In some embodiments, the IFNA2 variants include an S177X amino acid substitution in SEQ ID NO: 1, where X represents any amino acid. In some embodiments, the IFNA2 variant includes an S177D or S177R amino acid substitution in SEQ ID NO: 1.

In some embodiments, the IFNA2 variants include an N179X amino acid substitution in SEQ ID NO: 1, where X represents any amino acid. In some embodiments, the IFNA2 variant includes an N179G amino acid substitution in SEQ ID NO: 1.

In some embodiments, the IFNA2 variants include an S183X amino acid substitution in SEQ ID NO: 1, where X represents any amino acid. In some embodiments, the IFNA2 variant includes an S183W, S183F, S183M, S183E, S183Y, S183K, or S183L amino acid substitution in SEQ ID NO: 1.

In some embodiments, the IFNA2 variants include an R185X amino acid substitution in SEQ ID NO: 1, where X represents any amino acid. In some embodiments, the IFNA2 variant includes an R185D, R185E, or R185Q amino acid substitution in SEQ ID NO: 1.

In some embodiments, the IFNA2 variants include an S186X amino acid substitution in SEQ ID NO: 1, where X represents any amino acid. In some embodiments, the IFNA2 variant includes an S186L, S186I, or S186D amino acid substitution in SEQ ID NO: 1.

In some embodiments, the IFNA2 variants include an K187X amino acid substitution in SEQ ID NO: 1, where X represents any amino acid. In some embodiments, the IFNA2 variant includes an K187R, K187L, K187E, K187Y, K187V, K187M, K187F, K187I, K187W, or K187G amino acid substitution in SEQ ID NO: 1. In some embodiments, the IFNA2 variants include an E188X amino acid substitution in SEQ ID NO: 1, where X represents any amino acid. In some embodiments, the IFNA2 variant includes an E188I amino acid substitution in SEQ ID NO: 1.

In another aspect, the disclosure provides isolated IFNA2 variants, wherein the IFNA2 variants include two or more amino acid substitutions, wherein the two or more amino acid substitutions include a combination of substitutions selected from the group consisting of: M44N+A168R, L49P+S175P, L49P+S173H, L49P+M171W, L49P+M171G, L49P+S173R, M44N+S175P, L49P+M171V, L49P+M171K, L49P+M171S, L49P+S175R, L49P+M171R, L49P+L176G, L49P+M171Y, L49P+L176N, L49P+S173Q, L49P+L176H, L49P+L176R, L49P+M171A, L49P+M171T, L49P+L176A, L49P+M171F, M44N+S175K, L49P+S175K, L49P+L176E, L49P+E169T, L49P+M171L, L49P+L176Q, L49P+K187E, L49P+S173N, M44N+L176H, M44N+S175R, L49P+S177N, L49P+S173W, L49P+S177D, L49P+R185D, L49P+E169S, L49P+S175G, L49P+S177L, L49P+R172K, L49P+R185E, L49P+K187M, L49P+K187W, L49P+S159Q, M44N+L176Q, M44N+R185E, L49P+S175Q, L49P+S175M, L49P+E155V, L49P+K187D, L49P+S175W, L49P+L176V, L49P+S173L, M44N+S175G, L49P+R185G, L49P+R143W, L49P+I149T, L49P+R185L, L49P+R185S, L49P+S177H, L49P+K106D, M44N+R185N, L49P+K187V, L49P+K106W, L49P+K135T, M44N+V128I, M44N+R185D, L49P+K106P, L49P+S183M, L49P+S183D, L49P+K156A, L49P+S183L, M44N+K187D, L49P+R185M, L49P+R185N, M44N+A168L, L49P+S175L, L49P+R185Q, L49P+K187G, M44N+S177E, L49P+W163L, L49P+R185W, L49P+K187T, L49P+T109W, M44N+P160E, M44N+K106T, L49P+R185T, L49P+S175I, L49P+S173F, M44N+R185L, L49P+S183E, L49P+K187N, M44N+K187F, L49P+S159D, M44N+K106I, L49P+S175Y, L49P+L184N, L49P+I170L, L49P+V166S, L49P+K187A, L49P+S186D, L49P+L111F, L49P+K154Q, L49P+K106T, L49P+E136D, L49P+K106M, L49P+F107W, M44N+R185Y, M44N+V165I, M44N+K187W, L49P+R185I, M44N+R185T, M44N+R185V, M44N+Q113A, M44N+T178K, L49P+K156V, L49P+K187S, M44N+Q1141, L49P+T131E, M44N+S183E, L49P+Y152W, L49P+V128E, L49P+E155K, L49P+S175T, L49P+E155R, L49P+V166T, and L49P+T178F in the wild-type IFNA2 amino acid sequence (SEQ ID NO: 1).

In another aspect, the disclosure provides isolated fusion proteins including an antibody or a binding fragment thereof including an Fc domain; and a human IFNA2 variant, wherein the IFNA2 variant is covalently linked to the antibody or binding fragment thereof, and wherein the IFNA2 variant has decreased or no detectable binding to the human interferon-alpha/beta receptor beta (IFNAR2) as compared to the wild-type human IFNA2 polypeptide. In some embodiments, the IFNA2 variant has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

In another aspect, the disclosure provides isolated fusion proteins including an antibody or a binding fragment thereof including an Fc domain; and a human IFNA2 variant, wherein the IFNA2 variant is covalently linked to the Fc domain of the antibody or binding fragment thereof, and wherein the Fc domain has decreased or no detectable antibody dependent cellular cytotoxicity (ADCC) activity compared to the wild-type Fc. In some embodiments, the antibody or binding fragment thereof includes an amino acid sequence of SEQ ID NO: 4, 5, 6, 7, 8, or 9. In some embodiments, the antibody or binding fragment thereof has an isotype that is selected from the group consisting of NG, DANG, LALA, and LALA-PG. In some embodiments, the antibody or binding fragment thereof binds to an antigen selected from the group consisting of CD8, TIGIT, CLEC9A, LILRB4, LILRB2, PD-1, CD160, BTLA, and TNFRSF9.

In some embodiments, the antibody is selected from the group consisting of an anti-CTLA-4 antibody, an anti-CD3 antibody, an anti-CD4 antibody, an anti-CD8 antibody, an anti-4-1 BB antibody, an anti-PD-I antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-HVEM antibody, an anti-BTLA antibody, an anti-CD40 antibody, an anti-CD40L antibody, anti-CD47 antibody, an anti-CSFIR antibody, an anti-MARCO antibody, an anti-CXCR4 antibodies, an anti-VEGFR1 antibody, an anti-VEGFR2 antibody, an anti-TNFRI antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCR5 antibody, an anti-CD200R antibody, an anti-VISG4 antibody, an anti-CCR2 antibody, an anti-LILRB2 antibody, an anti-CXCR4 antibody, an anti-CD206 antibody, an anti-CD163 antibody, an anti-KLRGI antibody, an anti-FLT3 antibody, an anti-B7-H4 antibody, an anti-B7-H3 antibody, an KLRGI antibody, and an anti-GITR antibody, anti-CD160 antibody, anti-KLRD1 antibody, anti-KLRC1 antibody, anti-BTLA antibody, and anti-LILRB4 antibody.

In some embodiments, the IFNA2 variants are covalently linked to the antibody or binding fragment thereof by a polypeptide linker. In some embodiments, the polypeptide linker is selected from the group consisting of (G4S)2 and (G4S)3. In some embodiments, the polypeptide linker is a polypeptide tag selected from the group consisting of FLAG, MYC, HA, and 6×his.

In another aspect, the disclosure provides isolated cell lines that produce any one of the IFNA2 variants or any one of the fusion proteins disclosed herein. In some embodiments, the isolated cell lines are a CHO cell line or an HEK293 cell line.

In another aspect, the disclosure provides isolated nucleic acids encoding any one of the IFNA2 variants or any one of the fusion proteins disclosed herein.

In another aspect, the disclosure provides recombinant expression vectors including the nucleic acids encoding any one of the IFNA2 variants or any one of the fusion proteins disclosed herein.

In another aspect, the disclosure provides host cells including the isolated nucleic acids encoding any one of the IFNA2 variants or any one of the fusion proteins disclosed herein or the recombinant expression vector including the nucleic acid encoding any one of the IFNA2 variants or any one of the fusion proteins disclosed herein.

In another aspect, the disclosure provides pharmaceutical compositions including any one of the IFNA2 variants or any one of the fusion proteins disclosed herein, and a pharmaceutically acceptable carrier.

In another aspect, the disclosure provides methods of treating a disease in a subject in need thereof, the methods including administering to the subject an effective amount, e.g., a therapeutically effective amount, of a pharmaceutical composition as described herein including any one of the IFNA2 variants or any one of the fusion proteins disclosed herein, and a pharmaceutically acceptable carrier, such that one or more symptoms associated with the disease is ameliorated in the subject.

In some embodiments, the disease is cancer. In some embodiments, the cancer is a solid cancer or a liquid, e.g., blood-borne, cancer. In some embodiments, the solid cancer is selected from the group consisting of gastric cancer, small intestine cancer, sarcoma, head and neck cancer, thymic cancer, epithelial cancer, salivary cancer, liver cancer, biliary cancer, neuroendocrine tumors, stomach cancer, thyroid cancer, lung cancer, mesothelioma, ovarian cancer, breast cancer, prostate cancer, esophageal cancer, pancreatic cancer, glioma, renal cancer, bladder cancer, cervical cancer, uterine cancer, vulvar cancer, penile cancer, testicular cancer, anal cancer, choriocarcinoma, colorectal cancer, oral cancer, skin cancer, Merkel cell carcinoma, glioblastoma, brain tumor, bone cancer, eye cancer, and melanoma.

In some embodiments, the liquid cancer is selected from the group consisting of multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBY positive diffuse large B-cell lymphoma of the elderly, diffuse large B-cell lymphoma associated with inflammation, intravascular large B-cell lymphoma, ALKpositive large B-cell lymphoma, plasmablastic lymphoma, large B-cell lymphoma arising in HHVS-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other hematopoietic cells related cancer. In some embodiments, the cancer is relapsed, refractory, or metastatic.

In some embodiments, the methods further include administering an effective amount of a second therapeutic agent, optionally wherein the administration is separate, sequential, or simultaneous. In some embodiments, the second therapeutic agent is an antibody selected from the group consisting of an anti-CTLA-4 antibody, an anti-CD3 antibody, an anti-CD4 antibody, an anti-CD8 antibody, an anti-4-1BB antibody, ananti-PD-1 antibody, ananti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSFIR antibody, an anti-CSFI antibody, an anti-IL-7R antibody, an anti-MARCO antibody, an antiCXCR4 antibodies, an anti-VEGF antibody, an antiVEGFRI antibody, an anti-VEGFR2 antibody, an anti-TNFRI antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCRS antibody, an antiCD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRGI antibody, an anti-FLT3 antibody, an anti-B7-H4 antibody, an anti-B7-H3 antibody, an KLRGI antibody, a BTNIAl antibody, and an anti-GITR antibody.

In some embodiments, the second therapeutic agent is a cytokine, an immunocytokine, TNFa, a PAP inhibitor, an oncolytic virus, a kinase inhibitor, an ALK inhibitor, a MEK inhibitor, an IDO inhibitor, a GLSI inhibitor, a tyrosine kinase inhibitor, a CART cell or T cell therapy, a TLR agonist, or a tumor vaccine.

In another aspect, the disclosure provides pharmaceutical compositions including any one of the IFNA2 variants or any one of the fusion proteins disclosed herein, for use in the treatment of cancer, optionally wherein the cancer is a solid cancer or a liquid cancer and/or the cancer is relapsed, refractory, or metastatic. In some embodiments, the use is in combination with a second therapeutic agent, optionally wherein the combination is for administration simultaneously, concurrently, or simultaneously.

In another aspect, the disclosure provides isolated human interferon alpha-2 (IFNA2) variants, wherein the IFNA2 variants have decreased or no detectable binding to the human interferon-alpha/beta receptor beta 2 (IFNAR2) as compared to the wild-type human IFNA2 polypeptide, and wherein the IFNA2 variants comprise a polypeptide sequence of any one of SEQ ID NOs 10-416 and 439-538.

In some embodiments, the IFNA2 variants have a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2. In some embodiments, the isolated IFNA2 variants comprise a polypeptide sequence of any one of SEQ ID NOs 67, 79, 85, 99-101, 117, 118, 121, 159, 161, and 165.

In another aspect, the disclosure provides isolated nucleic acids encoding the IFNA2 variants.

In another aspect, the disclosure provides recombinant expression vectors comprising the isolated nucleic acids.

In another aspect, the disclosure provides host cells comprising the recombinant expression vectors.

In another aspect, the disclosure provides isolated fusion proteins including an antibody or binding fragment thereof, and an isolated human IFNA2 variant, wherein the IFNA2 variant is covalently linked to the antibody or binding fragment thereof, and wherein the IFNA2 variant has decreased or no detectable binding to the human interferon-alpha/beta receptor beta (IFNAR2) as compared to the wild-type human IFNA2 polypeptide, and wherein the IFNA2 variant comprises a polypeptide sequence of any one of SEQ ID NOs 10-416 and 439-538. In some embodiments, the antibody or binding fragment thereof comprises an Fc domain. In some embodiments, the isolated fusion proteins comprise a polypeptide sequence of any one of SEQ ID NOs 539-550 and 560-574. In some embodiments, the antibody is an anti-CD8 antibody. In some embodiments, the isolated fusion protein comprises a polypeptide sequence of any one of SEQ ID NOs 552-559.

In some embodiments, the isolated fusion proteins include an IFNA2 variant that has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2. In some embodiments, the IFNA2 variant is covalently linked to the antibody by a polypeptide linker. In some embodiments, the polypeptide linker is selected from the group consisting of (G4S)2 and (G4S)3. In some embodiments, the polypeptide linker is a polypeptide tag selected from the group consisting of FLAG, MYC, HA, and 6×his.

In another aspect, the disclosure provides isolated cell lines that produce the fusion proteins described herein.

In additional aspects, the disclosure provides isolated nucleic acid encoding the fusion proteins described herein.

In another aspect, the disclosure provides recombinant expression vectors comprising the nucleic acids described herein.

In other aspects, the disclosure provides host cells comprising the recombinant expression vectors described herein.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. 0., 1978, A model of evolutionary change in proteins-Matrices for detecting distant relationships. In Dayhoff, M. 0. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mo !. Biol. Evol. 4:406-425; Sncath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

As defined herein, a “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

As used herein, “protein-protein interaction” or “PPI” refers to physical contacts of high specificity established between two or more proteins (or polypeptides) as a result of biochemical events driven by electrostatic forces including the hydrophobic effect. Many are physical contacts with molecular associations between chains that occur in a cell or in a living organism in a specific biomolecular context. In some embodiments, the protein-protein interactions are strong enough to replace the function of the native sexual agglutination proteins. For example, it can be possible to couple mating efficiency to the interaction strength of a particular protein-protein interaction. In certain embodiments, the assay can characterize or determine protein-protein interactions between synthetic adhesion proteins (SAPs).

As used herein, a “synthetic adhesion protein” refers to any protein or polypeptide to be assayed for binding to or interacting with any other any protein or polypeptide. The proteins can be heterologous or exogenously expressed. Synthetic adhesion proteins are referred to as such because they are not typically associated with the adhesion required for agglutination as natively performed by the sexual agglutination proteins. In certain embodiments, the synthetic adhesion proteins have sufficiently strong interactions to allow agglutination in yeast where the native sexual agglutination proteins are not natively expressed. In some embodiments, the SAPs of the first and second expression cassettes of the first and second nucleic acid constructs, respectively, bind to a cell wall GPI anchored protein. In some embodiments, the SAPs can be fused to a cell wall GPI anchored protein or fused to a protein that forms a disulfide bond with a cell wall GPI anchored protein. In some embodiments, the SAP of the first expression cassette of the first nucleic acid construct is fused to the sexual agglutination protein Aga2, and the SAP of the first expression cassette of the second nucleic acid construct is fused to the sexual agglutination protein Aga2.

As used herein, “affinity” is a measure of the strength of the binding interaction between a single biomolecule to its ligand or binding partner. Affinity is usually measured and described using the equilibrium dissociation constant, Kn. The lower the Kn value, the greater the affinity between the protein and its binding partner. Affinity may be affected by hydrogen bonding, electrostatic interactions, hydrophobic and Van der Waals forces between the binding partners, or by the presence of other molecules, e.g., binding agonists or antagonists.

In some implementations, affinity may be described using arbitrary units, wherein a certain binding affinity within an assay, for example the binding affinity between two wildtype protein binding partners or the wild-type species of a first protein binding partner and the wild-type species of a second protein binding partner, is set to an arbitrary unit of 1.0 and binding affinities for other pairs of protein binding partners, for example the mutant species of a first protein binding partner and the mutant species of a second protein binding partner, are measured relative proportionally to that certain binding affinity.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a heatmap of AlphaSeq™ predicted affinities (log₁₀ Kd nM) for the IFNA2/IFNAR2 validation network. Strong binding is considered below 3, weak binding is considered between 3 and 4, no detectable binding is considered above 4.

FIG. 2A is a histogram of expression values for wild-type replicates of IFNA2. A value of 3 is the maximum possible expression in this assay and a value of 1 is the minimum possible expression.

FIG. 2B is a histogram of expression values for site-saturation mutagenesis (SSM) variants of IFNA2. A value of 3 is the maximum possible expression in this assay and a value of 1 is the minimum possible expression.

FIG. 3 is a scatter plot showing the relationship between expression (x-axis) and affinity for IFNAR2 in AlphaSeq™ (y-axis) for each wild-type replicate of IFNA2 in the IFNA2 SSM library.

FIG. 4A is a histogram of AlphaSeq™ affinity values for the replicates of wild-type IFNA2 binding to human IFNAR2.

FIG. 4B is a histogram of AlphaSeq™ affinity values for the full IFNA2 SSM binding to human IFNAR2.

FIG. 5A is a scatter plot showing the relationship between AlphaSeq™ affinity to human IFNAR2 (x-axis) and AlphaSeq™ affinity to cynomolgus IFNAR2 (y-axis) for each variant in the IFNA2 SSM library.

FIG. 5B is a scatter plot showing the relationship between AlphaSeq™ affinity to human IFNAR2 (x-axis) and AlphaSeq™ affinity to rat IFNAR2 (y-axis) for each variant in the IFNA2 SSM library.

FIG. 5C is a scatter plot showing the relationship between AlphaSeq™ affinity to human IFNAR2 (x-axis) and AlphaSeq™ affinity to mouse IFNAR2 (y-axis) for each variant in the IFNA2 SSM library.

FIG. 6 is a bar graph showing the affinity of wild-type IFNA2 to human, cynomolgus, rat and mouse IFNAR2 (black bars), along with two single mutants that show increased affinity to mouse and rat IFNAR2 (L49P: white bars; M44N: gray bars)

FIG. 7A is a histogram of AlphaSeq™ affinity values for the replicates of IFNA2_M44N binding to human IFNAR2.

FIG. 7B is a histogram of AlphaSeq™ affinity values for the replicates of IFNA2_M44N binding to mouse IFNAR2.

FIG. 7C is histogram of AlphaSeq™ affinity values for the full IFNA2_M44N SSM library binding to human IFNAR2.

FIG. 7D is a histogram of AlphaSeq™ affinity values for the full IFNA2_M44N SSM library binding to mouse IFNAR2.

FIG. 8A is a histogram of AlphaSeq™ affinity values for the replicates of IFNA2_L49P binding to human IFNAR2.

FIG. 8B is a histogram of AlphaSeq™ affinity values for the replicates of IFNA2_L49P binding to mouse IFNAR2.

FIG. 8C is histogram of AlphaSeq™ affinity values for the full IFNA2_L49P SSM library binding to human IFNAR2.

FIG. 8D is a histogram of AlphaSeq™ affinity values for the full IFNA2_L49P SSM library binding to mouse IFNAR2.

FIG. 9 is a scatter plot showing the relation between AlphaSeq™ affinity values and Kd rates measured by biolayer interferometry (BLI) for a subset of IFNA2 variants binding to wild-type human IFNAR2.

FIG. 10 is a graph that shows STAT1 phosphorylation (pSTAT1) in human peripheral blood mononuclear cells stimulated with IFNA2 variants or a negative control protein (Digoxin_scFv-Fc). Plotted is the median fluorescence intensity (MFI) of pSTATI in T cells. Dose-response curves were analyzed for EC50 calculations.

FIG. 11 is a scatter plot showing the relation between AlphaSeq™ affinity values and pSTAT1 EC50 in T cells, calculated from the dose-response curves shown in FIG. 10.

FIG. 12 is a graph of the dose-response curves for STATI phosphorylation as measured by flow cytometry for candidate IFNA2b immunocytokines in CD8 T cells.

FIG. 13 is a graph of the dose-response curves for STAT1 phosphorylation as measured by flow cytometry for candidate IFNA2b immunocytokines in CD4 T cells.

FIG. 14 is a graph of the dose-response curves for STATI phosphorylation as measured by flow cytometry for candidate IFNA2b immunocytokines in B cells.

FIG. 15 is a graph of the dose-response curves for STATI phosphorylation as measured by flow cytometry for candidate IFNA2b immunocytokines in monocytes.

FIG. 16 is a scatter plot showing the relation between the AlphaSeq™ affinity values and BLI-measured Kd for a second set of IFNA2 Fc fusion proteins comprising IFNA2 variants.

FIG. 17 is a scatter plot showing the correlation between the AlphaSeq™ affinity values and Phosflow-measured pSTAT1 EC50 in T cells for a second set of IFNA2 Fc fusion proteins comprising IFNA2 variants.

### DETAILED DESCRIPTION

The present disclosure provides detuned human interferon alpha-2 (IFNA2) variants and fusion proteins including those variants. By “detuned” is meant that the wildtype IFNA2 is engineered to have a reduced or weakened affinity for the interferon alpha/beta receptor 2 IFNAR2). Different modifications of the wildtype IFNA2 results in a large number of detuned IFNA2 variants.

In a therapeutic context, these detuned variants can be targeted to a particular immune cell type of interest by linking the detuned variants to an antibody or portion thereof against a specific cell surface marker. By reducing affinity for the receptor, the activity of the detuned cytokines is decreased for most cell types, e.g., in healthy tissues, which, in turn, decreases potential toxicity of the molecule when administered as a therapy. However, at the surface of the cell type targeted by the antibody, the residual activity of the cytokine is sufficient to bind its receptor and activate pro-inflammatory pathways leading to immune activation.

Cytokine signaling can trigger multifaceted and even opposing activities in different cell types, meaning that even if cytokine activity is limited specifically to the cells expressing the antibody targeted antigen activation of other cell types may be detrimental to the therapeutic efficacy of the cytokine. Targeting cytokine-dependent activation to only a subset of the immune cells present in a tumor, for example, activated T cells as opposed to all cells with a cytokine receptor, can allow improved targeting of the therapeutic effects of the molecule.

The compositions disclosed herein are human IFNA2 variants and fusion proteins made from these variants. As measured by, e.g., AlphaSeq™, and demonstrated in further detail in the Examples, the IFNA2 variants of the compositions and methods disclosed herein have decreased or no detectable binding to the IFNAR2 receptor, as compared to the wild-type human IFNA2 polypeptide or a wild-type IFNA2 fusion polypeptide. In some embodiments, these reduced affinity IFNA2 variants, when presented as an antibody fusion chimeric protein, are targeted selectively to desired cell types (those cells expressing the antibody target). Cell types that express the IFNAR2 receptor complex, but not the antibody target, are activated less, or not activated at all, compared to those cells which express both components. Accordingly, the IFNA2 variants and the IFNA2 fusion proteins of the compositions and methods disclosed herein selectively modulate the activation of cell subsets to promote biological activity, such as an anti-tumor activity, efficaciously and safely.

### Methods of Determining IFNA2 Variants

In some embodiments, measuring the affinity of the cytokine IFNA2 to its receptor interferon alpha/beta receptor 2 (IFNAR2) is performed using a high-throughput synthetic yeast agglutination protein-protein interaction (PPI) screening platform termed AlphaSeq™. Synthetic yeast agglutination relies on reprogramming yeast sexual agglutination—a naturally-occurring protein-protein interaction—to link protein-protein interaction strength with mating efficiency between a-type recombinant haploid yeast cells and a-type recombinant haploid yeast cells in liquid culture. For a screen of IFNA2 variants for binding affinity to IFNAR2 based on synthetic yeast agglutination, mating efficiency, represented by the number of diploid yeast cells formed in a turbulent liquid culture, is a proxy for affinity between IFNA2 and IFNAR2.

The AlphaSeq™ method is disclosed in, e.g., U.S. Pat. Nos. 10,988,759 and 11,136,573, which are incorporated herein by reference in their entireties. AlphaSeq™ can be used to perform library-by-library screens of a library of variants of one protein binding partner, e.g., IFNA2, against a library of variants of another protein binding partner, e.g., IFNAR2. AlphaSeq™ can be also used to perform a library-based screen of a library of variants of one protein binding partner, e.g., IFNA2, against a single species of another protein binding partner, e.g., wild-type human IFNAR2 or wild-type mouse IFNAR2. Each variant, i.e., protein of interest (POI) is assigned a unique oligonucleotide molecular barcode, and after diploid formation events, these protein-specific barcodes can be recombined and sequenced to identify the individual synthetic adhesion proteins (SAPs) that mediated the corresponding diploid formation event. Quantifying sequencing reads of unique barcode-barcode combinations acts as a proxy measure of the number of diploid formation events, and thus, PPI affinity.

In some embodiments, affinity between a library of IFNA2 variants and IFNAR2 is measured by the AlphaSeq™ method which is based on yeast sexual agglutination. In native yeast sexual agglutination, in a turbulent liquid culture, MATa and MATα haploid cells stick to one another due to the binding of sexual agglutinin proteins, which allows them to mate. The native sexual agglutinin proteins consist of Agal and Aga2, expressed by MATa cells, and Sagl, expressed by MATα cells. Agal and Sagl form GPI anchors with the cell wall and extend outside of the cell wall with glycosylated stalks. Aga2 is secreted by MATa cells and forms a disulfide bond with Agal. The interaction between Aga2 and Sagl is essential for wild-type sexual agglutination.

The native sexual agglutinin interaction can be replaced with an engineered one by expressing Agal in both mating types and fusing complementary binders to Aga2. In this case, a synthetic adhesion protein (SAP) comprises the fusion of Aga2 and the binder of interest, e.g., the library of IFNA2 variants expressed by cells of one mating type and IFNAR2 expressed by cells of the other mating type. Interaction of the SAPs therefore mediates adhesion, and subsequently the agglutination process. In some embodiments, instead of direct agglutination, it may be possible to express SAPs for a multivalent target, such that agglutination and mating only occurs in the presence of the target.

In some embodiments, each MATa and MATα haploid cell contains a SAP fused to Aga2 integrated into a target chromosome (for example, chromosome III). Upon mating, both copies of the target chromosome are present in the same diploid cell. In addition to the SAP/Aga2 cassette, each copy of the target chromosome has a unique primer binding site, one of a plurality of unique oligonucleotide barcodes operably linked to the particular SAP, and a lox recombination site.

The plurality of oligonucleotide barcodes can be synthesized and assembled with the library of SAP expression cassettes such that a single SAP species is operably linked to a plurality of unique oligonucleotide barcodes. Upon expression of Cre recombinase, a chromosomal translocation occurs at the lox sites, resulting in a juxtaposition of the primer binding sites and barcodes onto the same copy of the target chromosome. A PCR is then performed to amplify a region of the chromosome containing the barcodes from both SAPs, such that sequences comprising unique barcode-barcode pairs, each representing a diploid formation event, are amplified. In a batched mating, the result is a pool of fragments, each containing the unique barcode-barcode pair associated with two SAPs that were responsible for the single diploid formation event. Paired-end next generation sequencing is then used to match the barcodes and determine the number of diploid formation events mediated by that SAP pair.

In some embodiments, the a-agglutinin, Sag 1, is knocked out in MATa cells to eliminate native agglutination. MATa and MATα cells are able to synthesize lysine or leucine, respectively. Diploids can then be selected for in media lacking both amino acids. MATa cells express ZEV4, a BE inducible transcription factor that activates Cre recombinase expression in diploid cells. MATa and MATα cells express mCherry and mTurquoise, respectively, for identification of strain types with flow cytometry. MATa and MATα cells constitutively express Agal along with a uniquely barcoded SAP fused to Aga2. When Cre recombinase expression is induced in diploids with βE, a chromosomal translocation at lox sites consolidates both SAP-Aga2 fusion expression cassettes onto the same chromosome. A single fragment containing the unique barcode-barcode sequence associated with that diploid formation event is then amplified by PCR with primers annealing to Pf and Pr (primers specific to the primers from the first and second nucleic acid constructs integrated at the genomic target site) and sequenced to quantify the number of diploid formation events and identify the interacting SAP pair.

In some embodiments, a CRE recombinase translocation scheme is utilized for high throughput analysis for interactions between synthetic adhesion proteins from a library to library screen, or, e.g., the library of IFNA2 variants expressed by cells of one mating type and IFNAR2 expressed by cells of the other mating type. When CRE recombinase expression is induced in diploids with βE, a chromosomal translocation at lox sites consolidates both SAP-Aga2 expression cassettes onto the same chromosome. A single fragment containing the unique barcode-barcode sequence associated with that diploid formation event is then amplified by PCR with primers annealing to primer binding sites from each of the first and second nucleic acid constructs and sequenced (for example, using a paired end analysis of next generation sequencing) to quantify the number of diploid formation events and identify the interacting SAP pair, e.g., a unique variant of IFNA2 and IFNAR2, thereby yielding an estimation of the affinity between the variant of IFNA2 and IFNAR2.

In some embodiments, the methods for measuring the affinity of a cytokine and its receptor include a library of cytokine variants, e.g., variants of IFNA2, comprising a plurality of cytokine variants and a receptor or library of receptor variants, e.g., IFNAR2 or variants of IFNAR2. The cytokine variants and the receptor or library of receptor variants can be user-designated or randomly added mutants of a protein and the wild-type protein. In some embodiments, the amino acid substitutions may be generated by site saturation mutagenesis (SSM) to produce an SSM library of the cytokine and the receptor. In some embodiments, the variants and the receptor variants can be generated by alanine scanning. In some embodiments, the cytokine variants and the receptor variants can be generated by random mutagenesis, such as with error prone PCR, or another method to introduce variation into the amino acid sequence of the expressed protein. The cytokine variant library comprising a plurality of cytokine variants and the receptor variant library comprising a plurality of receptor variants are assayed for binding affinity, such that affinity is measured for interaction between each of the plurality of variants and the wild-type receptor or each of the plurality of receptor variants individually, in a parallelized high-throughput manner.

In some embodiments, the cytokine variants and the receptor or receptor variants are full-length proteins. In some embodiments, the cytokine variants and the receptor or receptor variants are truncated proteins. In some embodiments, the cytokine variants and the receptor or receptor variants are fusion proteins. In some embodiments, the cytokine variants and the receptor or receptor variants are tagged proteins. Tagged proteins include proteins that are epitope tagged, e.g., FLAG-tagged, HA-tagged, His-tagged, Myc-tagged, among others known in the art. The cytokine variants and the receptor or receptor variants can each be any of the following: a full-length protein, truncated protein, fusion protein, tagged protein, or combinations thereof.

In some embodiments, the methods for measuring the affinity of a cytokine and its receptor include bio-layer interferometry (BLI). BLI measures kinetics and biomolecular interactions on a basis of wave interference. To prepare for BLI analysis between two unique biomolecules, the ligand is first immobilized onto a bio compatible biosensor while the analyte is in solution. After this, the biosensor tip is dipped into the solution and the target molecule will begin to associate with the analyte, producing a layer on top of the biosensor tip. This creates two separate surfaces: the substrate itself, and the substrate interacting with the molecule immobilized on the biosensor tip. This can create a thin-film interference, in which the created layer acts as a thin film bound by these two surfaces. White light from a tungsten lamp is shone onto the biosensor tip and reflected off both surfaces, creating two unique reflection patterns with different intensities.

In some cases, it is possible that cytokine variants, e.g., IFNA2 variants, can be measured by various affinity determination methods, e.g., AlphaSeq™ and/or BLI to have no detectable binding to the cytokine's receptor, e.g., IFNAR2, while still inducing signaling in cells in vitro or in vivo. In such cases, a cytokine variant exhibiting no detectable binding to its receptor as measured by AlphaSeq™ or BLI, or a fusion protein comprising the cytokine variant, can potentially have therapeutic efficacy.

### IFNA2 Variants

IFNA2 variants (e.g., human IFNA2 variants) as described herein have decreased or no detectable binding to the IFNAR2 receptor and/or reduced interaction between IFNA2 and the IFNAR2 receptor, as compared to the wild-type human IFNA2 polypeptide, as described in further detail in the Examples section. The IFNA2 variants provided herein are recited in Tables 1-5 as comprising amino acid substitutions numbered according to the wild-type sequence of IFNA2:

(SEQ ID NO: 1)
MALTFALLVALLVLSCKSSCSVGCDLPQTHSLGSRRTLMLLAOMR
RISLFSCLKDRHDFGFPQEEFGNQFOKAETIPVLHEMIQQIFNLF
STKDSSAAWDETLLDKFYTELYQQLNDLEACVIQGVGVTETPLMK
EDSILAVRKYFQRITLYLKEKKYSPCAWEVVRAEIMRSFSLSTNL
QESLRSKE

For example, a variant with the name “IFNA2b_V165Y” has an amino acid substitution in SEQ ID NO: 1 at location 165, where the existing valine is replaced with tyrosine.

Tables 1-3 also recite affinities between IFNA2 and the human interferon alpha/beta receptor 2 (IFNAR2) as measured by AlphaSeq™ (in nM). A value of 3 is the maximum possible expression in this assay and a value of 1 is the minimum possible expression. The amino acid sequence of human interferon alpha/beta receptor 2 (IFNAR2) is provided below:

(SEQ ID NO: 2)
MLLSQNAFIFRSLNLVLMVYISLVFGISYDSPDYTDESCTFKISL
RNFRSILSWELKNHSIVPTHYTLLYTIMSKPEDLKVVKNCANTTR
SFCDLTDEWRSTHEAYVTVLEGFSGNTTLFSCSHNFWLAIDMSFE
PPEFEIVGFTNHINVMVKFPSIVEEELQFDLSLVIEEQSEGIVKK
HKPEIKGNMSGNFTYIIDKLIPNTNYCVSVYLEHSDEQAVIKSPL
KCTLLPPGQESESAESAKIGGIITVFLIALVLTSTIVTLKWIGYI
CLRNSLPKVLNFHNFLAWPFPNLPPLEAMDMVEVIYINRKKKVWD
YNYDDESDSDTEAAPRTSGGGYTMHGLTVRPLGQASATSTESQLI
DPESEEEPDLPEVDVELPTMPKDSPOOLELLSGPCERRKSPLODP
FPEEDYSSTEGSGGRITFNVDLNSVELRVLDDEDSDDLEAPLMLS
SHLEEMVDPEDPDNVOSNHLLASGEGTOPTFPSPSSEGLWSEDAP
SDQSDTSESDVDLGDGYIMR

As shown in Table 1 below, disclosed herein are 280 IFNA2 variants comprising single amino acid substitutions, with amino acid substitutions throughout the length of the wild-type IFNA2 polypeptide (SEQ ID NO: 1) with reduced affinity for human IFNAR2. Any variant found to bind human IFNAR2 with an affinity weaker than 100 nM (log₁₀ nM=2) as measured by AlphaSeq™ and found to have an average expression bin>2.0 is included in Table 1. Table 1 is sorted in order of descending Kd, with the highest values representing the lowest affinity of IFNA2 for human IFNAR2. Table 1 also provides the polypeptide sequences of the 280 IFNA2 variants comprising single amino acid substitutions as SEQ ID NOs 10-289. The polypeptide sequences describe the mature IFNA2b protein, which does not include a 23-amino acid signal peptide that is cleaved from the N-terminus to produce the mature protein.

TABLE 1
IFNA2 Variants Comprising Single Amino Acid
Substitutions with Detuned Affinity to Human IFNAR2

| | Affinity | | | |
|---|---|---|---|---|
| | to human | Average | | SEQ |
| | IFNAR2 | expression | | ID |
| Variant | (nM) | bin | Polypeptide Sequence | NO |
| IFNA2b_ | 175684 | 2.08 | CDLPQTHSLGSRRTLMLLAQMRRISLFSCLKDRHDFGF | 10 |
| V165Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEYVRAEIMRSFS | |
| | | | LSTNLQESLRSKE | |
| IFNA2b_ | 166760 | 2.62 | CDLPQTHSLGSRRTLMLLAQMRRISLFSCLKDDHDFGF | 11 |
| R56D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 165921 | 2.04 | CDLPQTHSLGSRRTLMLLAQMRRISLFSCLKDRHDQGF | 12 |
| F59Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 134718 | 2.29 | CDLPQTHSLGSRRTLMLLAQMRRISLDSCLKDRHDFGF | 13 |
| F50D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 129330 | 2.10 | CDLPQTHSLGSRRTLMLHAQMRRISLFSCLKDRHDFGF | 14 |
| L41H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 126400 | 2.28 | CDLPQTHSLGSRRTLMLLAQMRRISLFSCLKDRHDFGF | 15 |
| A168R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRREIMRSES | |
| | | | LSTNLQESLRSKE | |
| IFNA2b_ | 125833 | 2.35 | CDLPQTHSLGSRRTLMLLAQMRRISLFSCLKDRHDEGF | 16 |
| F59E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 98339 | 2.37 | CDLPQTHSLGSRRTLMLLAQMRRISLFSCLKDRHDFGF | 17 |
| V165E | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEEVRAEIMRSFS | |
| | | | LSTNLQESLRSKE | |
| IFNA2b_ | 96394 | 2.48 | CDLPQTHSLGSRRTLMLLAQMRRISLESCLKDGHDFGF | 18 |
| R56G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 94074 | 2.32 | CDLPQTHSLGSRRTLMLLAQMRRISLFSCDKDRHDFGF | 19 |
| L53D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 92621 | 2.21 | CDLPQTHSLGSRRTLMLLAQMRRISLFSCEKDRHDFGF | 20 |
| L53E | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 91022 | 2.16 | CDLPQTHSLGSRRTLMLLAQMRRISLFSCGKDRHDFGF | 21 |
| L53G | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 88720 | 2.29 | CDLPQTHSLGSRRTLMLLAQMRRISLFSCAKDRHDFGF | 22 |
| L53A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 87906 | 2.38 | CDLPQTHSLGSRRTLMLLAQMRRISLFSCLKDRHDFGF | 23 |
| M171R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIRRSFS | |
| | | | LSTNLQESLRSKE | |
| IFNA2b_ | 86786 | 2.37 | CDLPQTHSLGSRRTLMLLAQMRRISLFSCLKDKHDFGF | 24 |
| R56K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 82118 | 2.15 | CDLPQTHSLGSRRTLMLLAQMRRISLFSCLKDRHDFGF | 25 |
| V165H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEHVRAEIMRSFS | |
| | | | LSTNLQESLRSKE | |
| IFNA2b_ | 80784 | 2.27 | CDLPQTHSLGSRRTLMLLAQMRRISLFSCLKDRHDFGF | 26 |
| V165K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEKVRAEIMRSFS | |
| | | | LSTNLQESLRSKE | |
| IFNA2b_ | 79034 | 2.16 | CDLPQTHSLGSRRTLMLLAQMRRISLFSCLKDRHDFGF | 27 |
| M171S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEISRSFS | |
| | | | LSTNLQESLRSKE | |
| IFNA2b_ | 76944 | 2.25 | CDLPQTHSLGSRRTLMLDAQMRRISLFSCLKDRHDFGF | 28 |
| L41D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 73763 | 2.19 | CDLPQTHSLGSRRTLMLLAQMRRISLFSCLKDRHDIGF | 29 |
| F59I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 69054 | 2.38 | CDLPQTHSLGSRRTLMLLAQMRRISLFSCLKDNHDFGF | 30 |
| R56N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 65172 | 2.27 | CDLPQTHSLGSRRTLMLLAQMRRISLFSCLKDRPDFGF | 31 |
| H57P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 64832 | 2.12 | CDLPQTHSLGSRRTLMLLAQMRRISLESCLKDRHDFGF | 32 |
| R167W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVWAEIMRSFS | |
| | | | LSTNLQESLRSKE | |
| IFNA2b_ | 63555 | 2.32 | CDLPQTHSLGSRRTLMLLAQMRRISLESCLKDRHDFGF | 33 |
| A168H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRHEIMRSES | |
| | | | LSTNLQESLRSKE | |
| IFNA2b_ | 61610 | 2.04 | CDLPQTHSLGSRRTLMLLAQMRRISLFSCLKDRHDNGF | 34 |
| F59N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 61176 | 2.32 | CDLPQTHSLGSRRTLMLLAQMRRISLFSCLKDRHDFGF | 35 |
| M171T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEITRSFS | |
| | | | LSTNLQESLRSKE | |
| IFNA2b_ | 55443 | 2.22 | CDLPQTHSLGSRRTLMLLAQMRRISLFSCLKDRHDFGF | 36 |
| V165W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEWVRAEIMRSES | |
| | | | LSTNLQESLRSKE | |
| IFNA2b_ | 52734 | 2.21 | CDLPQTHSLGSRRTLMLLAQMRRISHFSCLKDRHDFGF | 37 |
| L49H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 52584 | 2.35 | CDLPQTHSLGSRRTLMLLAQMRRISLFSCLKDRHDFGF | 38 |
| V165F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEFVRAEIMRSFS | |
| | | | LSTNLQESLRSKE | |
| IFNA2b_ | 51981 | 2.37 | CDLPQTHSLGSRRTLMLLAQMRRISLFSCNKDRHDFGF | 39 |
| L53N | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 51955 | 2.19 | CDLPQTHSLGSRRTLMLLAQMRRISLFSCLKDRHDAGF | 40 |
| F59A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 50815 | 2.43 | CDLPQTHSLGSRRTLMLLAQMRRISLFSCLKDRHDFGF | 41 |
| A168K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRKEIMRSFS | |
| | | | LSTNLQESLRSKE | |
| IFNA2b_ | 50691 | 2.50 | CDLPQTHSLGSRRTLMLLAQMRRISLFSCLKDRHDFGF | 42 |
| R172D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMDSFS | |
| | | | LSTNLQESLRSKE | |
| IFNA2b_ | 49479 | 2.00 | CDLPQTHSLGSRRTLMLLAQMRRISLFSCLKDRHDFGF | 43 |
| R167I | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVIAEIMRSFS | |
| | | | LSTNLQESLRSKE | |
| IFNA2b_ | 48574 | 2.06 | CDLPQTHSLGSRRTLMLKAQMRRISLFSCLKDRHDFGF | 44 |
| L41K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 46593 | 2.18 | CDLPQTHSLGSRRTLMLLAQMRRISLFSCLKDRHDGGF | 45 |
| F59G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 44697 | 2.18 | CDLPQTHSLGSRRTLMLLAQMRRISLFSCLKDVHDFGF | 46 |
| R56V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 44067 | 2.02 | CDLPQTHSLGSRRTLMLLAQMRRISLFSCLKDRHDFGF | 47 |
| R172W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMWSFS | |
| | | | LSTNLQESLRSKE | |
| IFNA2b_ | 43611 | 2.38 | CDLPQTHSLGSRRTLMLLAQMRRISLFSCLKDTHDFGF | 48 |
| R56T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 43249 | 2.07 | CDLPQTHSLGSRRTLMLLAQMRRISIFSCLKDRHDFGF | 49 |
| L49I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 41999 | 2.23 | CDLPQTHSLGSRRTLMLLAQMRRISLFSCLKDRHDFGF | 50 |
| R172N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMNSES | |
| | | | LSTNLQESLRSKE | |
| IFNA2b_ | 40624 | 2.39 | CDLPQTHSLGSRRTLMLLAQMRRISLFSCLKDRHDFGF | 51 |
| M171N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEINRSFS | |
| | | | LSTNLQESLRSKE | |
| IFNA2b_ | 38419 | 2.46 | CDLPQTHSLGSRRTLMLLAQMRRISKFSCLKDRHDFGF | 52 |
| L49K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 38210 | 2.36 | CDLPQTHSLGSRRTLMLLAQMRRISLFSCLKDRHDFGF | 53 |
| V165Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEQVRAEIMRSFS | |
| | | | LSTNLQESLRSKE | |
| IFNA2b_ | 36997 | 2.30 | CDLPQTHSLGSRRTLMLLAQMRRISLFSCVKDRHDFGF | 54 |
| L53V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 34978 | 2.13 | CDLPQTHSLGSRRTLMLLAQMRRISLFSCSKDRHDFGF | 55 |
| L53S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 34802 | 2.27 | CDLPQTHSLGSRRTLMLGAQMRRISLFSCLKDRHDFGF | 56 |
| L41G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 34146 | 2.41 | CDLPQTHSLGSRRTLMLLAQMRRISLFSCLKDAHDFGF | 57 |
| R56A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 33225 | 2.05 | CDLPQTHSLGSRRTLMLLAQMRRISLFSCLKDRHDTGF | 58 |
| F59T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 29487 | 2.09 | CDLPQTHSLGSRRTLMLLAQMRRISLFSCLKDLHDFGF | 59 |
| R56L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 28783 | 2.12 | CDLPQTHSLGSRRTLMLLAQMRRISLFSCLKDRHDFGF | 60 |
| S173K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRKFS | |
| | | | LSTNLQESLRSKE | |
| IFNA2b_ | 28741 | 2.06 | CDLPQTHSLGSRRTLMLLAQMRRISLFSCLKDHHDFGF | 61 |
| R56H | | | PQEEFGNQFQKAETIPVLHEMIQQIENLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 28607 | 2.41 | CDLPQTHSLGSRRTLMLLAQMRRISLFSCLKDRHDFGF | 62 |
| R167M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVMAEIMRSFS | |
| | | | LSTNLQESLRSKE | |
| IFNA2b_ | 27318 | 2.16 | CDLPQTHSLGSRRTLMLLAQMRRISLFSCLKDRHDFGF | 63 |
| R172A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMASES | |
| | | | LSTNLQESLRSKE | |
| IFNA2b_ | 27280 | 2.27 | CDLPQTHSLGSRRTLMLLAQMRRISLFSCLKDRHDFGF | 64 |
| R167S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVSAEIMRSFS | |
| | | | LSTNLQESLRSKE | |
| IFNA2b_ | 26443 | 2.21 | CDLPQTHSLGSRRTLMLLAQMRRISLFSCLKDRHDFGF | 65 |
| R172V | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMVSFS | |
| | | | LSTNLQESLRSKE | |
| IFNA2b_ | 26316 | 2.11 | CDLPQTHSLGSRRTLMLLAQMRRISLFSCLKDRHDFGF | 66 |
| V165L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWELVRAEIMRSFS | |
| | | | LSTNLQESLRSKE | |
| IFNA2b_ | 24600 | 2.62 | CDLPQTHSLGSRRTLMLLAQMRRPSLFSCLKDRHDFGF | 67 |
| I47P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 23892 | 2.30 | CDLPQTHSLGSRRTLMLLAQMRRISLFSCLKDRHDFGF | 68 |
| S173R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRRFS | |
| | | | LSTNLQESLRSKE | |
| IFNA2b_ | 22135 | 2.04 | CDLPQTHSLGSRRTLMLLAQMRRISLFSCLKDRHDFGF | 69 |
| R167E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVEAEIMRSFS | |
| | | | LSTNLQESLRSKE | |
| IFNA2b_ | 21491 | 2.46 | CDLPQTHSLGSRRTLMLLAQMRRISLFSCLKDRHDFGF | 70 |
| M171A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIARSES | |
| | | | LSTNLQESLRSKE | |
| IFNA2b_ | 21334 | 2.40 | CDLPQTHSLGSRRTLMLLAQMRRISLFSCLKDRHDFGF | 71 |
| M171Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIYRSFS | |
| | | | LSTNLQESLRSKE | |
| IFNA2b_ | 20819 | 2.17 | CDLPQTHSLGSRRTLMLLAQMRRISLFSCLKDRHDFGF | 72 |
| M171W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIWRSFS | |
| | | | LSTNLQESLRSKE | |
| IFNA2b_ | 20710 | 2.29 | CDLPQTHSLGSRRTLMLLAQMRRISLFSCLKDRHDFGF | 73 |
| V165M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEMVRAEIMRSFS | |
| | | | LSTNLQESLRSKE | |
| IFNA2b_ | 19164 | 2.37 | CDLPQTHSLGSRRTLMLLAQMRRISLFSCLKDRHDFGF | 74 |
| M171F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIFRSFS | |
| | | | LSTNLQESLRSKE | |
| IFNA2b_ | 19085 | 2.00 | CDLPQTHSLGSRRTLMLLAQMRRISLFSCLKDRHDFGF | 75 |
| A168Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRYEIMRSFS | |
| | | | LSTNLQESLRSKE | |
| IFNA2b_ | 15489 | 2.16 | CDLPQTHSLGSRRTLMLLAQMRRISLFSCLKDRHDFGF | 76 |
| V165S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWESVRAEIMRSFS | |
| | | | LSTNLQESLRSKE | |
| IFNA2b_ | 14751 | 2.24 | CDLPQTHSLGSRRTLMLLAQMRRISLFSCLKDRHDFGF | 77 |
| V165R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWERVRAEIMRSFS | |
| | | | LSTNLQESLRSKE | |
| IFNA2b_ | 10833 | 2.41 | CDLPQTHSLGSRRTLMLLAQMRRISLFSCLKDRHDFGF | 78 |
| S175R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFR | |
| | | | LSTNLQESLRSKE | |
| IFNA2b_ | 10757 | 2.43 | CDLPQTHSLGSRRTLMLLAQMRRISLFSCLKDRHDFGF | 79 |
| A168G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRGEIMRSFS | |
| | | | LSTNLQESLRSKE | |
| IFNA2b_ | 10734 | 2.40 | CDLPQTHSLGSRRTLMLLAQMRRISLFSCLKDRHDFGF | 80 |
| R172G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMGSES | |
| | | | LSTNLQESLRSKE | |
| IFNA2b_ | 10717 | 2.46 | CDLPQTHSLGSRRTLMLLAQMRRISLFSCLKDRHDFGF | 81 |
| M171K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIKRSFS | |
| | | | LSTNLQESLRSKE | |
| IFNA2b_ | 9929 | 2.44 | CDLPQTHSLGSRRTLMLLAQMRRISLFSCLKDRHDFGF | 82 |
| M171E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIERSFS | |
| | | | LSTNLQESLRSKE | |
| IFNA2b_ | 9510 | 2.33 | CDLPQTHSLGSRRTLMLLAQMRPISLFSCLKDRHDFGF | 83 |
| R46P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 9254 | 2.47 | CDLPQTHSLGSRRTLMLLAQMRDISLFSCLKDRHDFGF | 84 |
| R46D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 8755 | 2.12 | CDLPQTHSLGSRRTLMLLAQMRRISLFSCLKDRHDFGF | 85 |
| R172T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMTSFS | |
| | | | LSTNLQESLRSKE | |
| IFNA2b_ | 8584 | 2.49 | CDLPQTHSLGSRRTLMLLAQMRRISLFSCLKDRHDFGF | 86 |
| R167L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVLAEIMRSFS | |
| | | | LSTNLQESLRSKE | |
| IFNA2b_ | 6708 | 2.06 | CDLPQTHSLGSRRTLMLLAQMRRISLESCLKDRHDYGF | 87 |
| F59Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 6510 | 2.05 | CDLPQTHSLGSRRTLMLLAQMRRISLFSCLKDRHDFGF | 88 |
| L176N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | NSTNLQESLRSKE | |
| IFNA2b_ | 5745 | 2.20 | CDLPQTHSLGSRRTLMLLAQMRRISLFSCLKDRHDFGF | 89 |
| E169T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRATIMRSFS | |
| | | | LSTNLQESLRSKE | |
| IFNA2b_ | 5479 | 2.22 | CDLPQTHSLGSRRTLMLLAQMRRISLFSCLKDRHDFGF | 90 |
| L176G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | GSTNLQESLRSKE | |
| IFNA2b_ | 5478 | 2.61 | CDLPQTHSLGSRRTLMLLAQMRRISLFSCLKDRHDFGF | 91 |
| R167V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVVAEIMRSFS | |
| | | | LSTNLQESLRSKE | |
| IFNA2b_ | 5071 | 2.19 | CDLPQTHSLGSRRTLMLLAQMRRISLFSCLKDRHDFGF | 92 |
| R172S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMSSFS | |
| | | | LSTNLQESLRSKE | |
| IFNA2b_ | 4507 | 2.31 | CDLPQTHSLGSRRTLMLLAQMRRISLFSCLKDRHDFGF | 93 |
| S173H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRHFS | |
| | | | LSTNLQESLRSKE | |
| IFNA2b_ | 4143 | 2.14 | CDLPQTHSLGSRRTLMLLAQMRRISLFSCLKDRHDFGF | 94 |
| M171G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIGRSFS | |
| | | | LSTNLQESLRSKE | |
| IFNA2b_ | 3993 | 2.17 | CDLPQTHSLGSRRTLMLLAQMRRISVFSCLKDRHDFGF | 95 |
| L49V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 3391 | 2.25 | CDLPQTHSLGSRRTLMLLAQMRRISLNSCLKDRHDFGF | 96 |
| F50N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 3372 | 2.51 | CDLPQTHSLGSRRTLMLLAQMRRISLFSCLKDRHDFGF | 97 |
| R167A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVAAEIMRSFS | |
| | | | LSTNLQESLRSKE | |
| IFNA2b_ | 3254 | 2.31 | CDLPQTHSLGSRRTLMLLAQMRRDSLFSCLKDRHDFGF | 98 |
| I47D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 3193 | 2.09 | CDLPQTHSLGSRRTLMLLAQMRFISLFSCLKDRHDFGF | 99 |
| R46F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 3055 | 2.34 | CDLPQTHSLGSRRTLMLLAQMRRISLFSCLKDRHDFGF | 100 |
| S175K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFK | |
| | | | LSTNLQESLRSKE | |
| IFNA2b_ | 3029 | 2.01 | CDLPQTHSLGSRRTLMLLAQMRRISLFSCLKDRHDFGF | 101 |
| M171L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEILRSFS | |
| | | | LSTNLQESLRSKE | |
| IFNA2b_ | 2783 | 2.11 | CDLPQTHSLGSRRTLMLLAQMRRISLFSCLKDRHDFGF | 102 |
| V126W | | | PQEEFGNQFQKAETIPVLHEMIQQIFNLFSTKDSSAAW | |
| | | | DETLLDKFYTELYQQLNDLEACVIQGWGVTETPLMKED | |
| | | | SILAVRKYFQRITLYLKEKKYSPCAWEVVRAEIMRSFS | |
| | | | LSTNLQESLRSKE | |
| IFNA2b_ | 2457 | 2.04 | CDLPQTHSLGSRRTLMLLAQMRRISLFSCLKDRHDFGF | 103 |
| S175M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFM | |
| | | | LSTNLQESLRSKE | |
| IFNA2b_ | 2358 | 2.35 | CDLPQTHSLGSRRTLMLLAQMRRISLFSCLKDRHDFGF | 104 |
| L176H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | HSTNLQESLRSKE | |
| IFNA2b_ | 2284 | 2.35 | CDLPQTHSLGSRRTLMLLAQMRRISLFSCLKDRHDFGF | 105 |
| A168F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRFEIMRSES | |
| | | | LSTNLQESLRSKE | |
| IFNA2b_ | 2133 | 2.14 | CDLPQTHSLGSRRTLMLLAQMRRISLFSCLKDRHDFGF | 106 |
| M171I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIIRSES | |
| | | | LSTNLQESLRSKE | |
| IFNA2b_ | 2115 | 2.09 | CDLPQTHSLGSRRTLMLLAQMRRISLFSCLKDRHDFGF | 107 |
| R167G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVGAEIMRSFS | |
| | | | LSTNLQESLRSKE | |
| IFNA2b_ | 2052 | 2.32 | CDLPQTHSLGSRRTLMLLAQMRRISLFSCLKDRHDFGF | 108 |
| V165N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWENVRAEIMRSFS | |
| | | | LSTNLQESLRSKE | |
| IFNA2b_ | 2015 | 2.00 | CDLPQTHSLGSRRTLMLLAQMRYISLFSCLKDRHDFGF | 109 |
| R46Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 1871 | 2.32 | CDLPQTHSLGSRRTLMLLAQMRRISLFSCLKDRHDFGF | 110 |
| M171V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIVRSES | |
| | | | LSTNLQESLRSKE | |
| IFNA2b_ | 1824 | 2.01 | CDLPQTHSLGSRRTHMLLAQMRRISLFSCLKDRHDFGF | 111 |
| L38H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 1591 | 2.07 | CDLPQTHSLGSRRTLMLLAQMRRISLFSCLKDRHDFGF | 112 |
| L176A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | ASTNLQESLRSKE | |
| IFNA2b_ | 1455 | 2.03 | CDLPQTHSLGSRRTLMLAAQMRRISLFSCLKDRHDFGF | 113 |
| L41A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 1398 | 2.13 | CDLPQTHSLGSRRTLMLLAQMRRISLFSCLKDRHDFGF | 114 |
| L176P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | PSTNLQESLRSKE | |
| IFNA2b_ | 1305 | 2.42 | CDLPQTHSLGSRRTLMLLAQMRRISLFSCLKDRHDFGF | 115 |
| L176D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | DSTNLQESLRSKE | |
| IFNA2b_ | 1225 | 2.17 | CDLPQTHSLGARRTLMLLAQMRRISLFSCLKDRHDFGF | 116 |
| S34A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 1206 | 2.33 | CDLPQTHSLGSRRTLMLLAQMRRISLFSCLKDRHDFGF | 117 |
| S183W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQEWLRSKE | |
| IFNA2b_ | 1182 | 2.05 | CDLPQTHSLGSRRTLMLLAQMRRISLFSCLKDRHDFGF | 118 |
| N116W | | | PQEEFGNQFQKAETIPVLHEMIQQIENLESTKDSSAAW | |
| | | | DETLLDKFYTELYQQLWDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 1003 | 2.24 | CDLPQTHSLGSRRTLMLLAQMRRISLFSCLKDRHDFGF | 119 |
| S173E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMREFS | |
| | | | LSTNLQESLRSKE | |
| IFNA2b_ | 991 | 2.15 | CDLPQTHSLGSRRTLMLLAQMRRISLFSCLKDRHDFGF | 120 |
| S175L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFL | |
| | | | LSTNLQESLRSKE | |
| IFNA2b_ | 863 | 2.15 | CDLPQTHSLGSRRTLMLLAQMRRISLGSCLKDRHDFGF | 121 |
| F50G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 861 | 2.11 | CDLPQTHSLGSRRTLMLLAQMRRISLFSCLKDRHDFGF | 122 |
| M82W | | | PQEEFGNQFQKAETIPVLHEWIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 854 | 2.30 | CDLPQTHSLGSRRTLMLLAQMRRISLFSCLKDRHDFGF | 123 |
| L176R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | RSTNLQESLRSKE | |
| IFNA2b_ | 831 | 2.22 | CDLPQTHSLGSRRTLMLLAQMRRISLESCLKDRHDFGF | 124 |
| S175P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFP | |
| | | | LSTNLQESLRSKE | |
| IFNA2b_ | 781 | 2.13 | CDLPQTHSLGSRRTLMLLAQMRRISLFSCLKDRHDFGF | 125 |
| E188I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKI | |
| IFNA2b_ | 777 | 2.08 | CDLPQTHSLGSRRTLMLLAQMRRISLFSCLKDRHDFGF | 126 |
| S175I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFI | |
| | | | LSTNLQESLRSKE | |
| IFNA2b_ | 774 | 2.12 | CDLPQTHSLGSRRTLMLLAQMRRISLFSCLKTRHDFGF | 127 |
| D55T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 689 | 2.26 | CDLPQTHSLGSRRTLMLLAQMRRISYFSCLKDRHDFGF | 128 |
| L49Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 688 | 2.28 | CDLPQTHSLGSRRTLMLLAQMRRISLFSCLKDRHDFGF | 129 |
| R172Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMYSFS | |
| | | | LSTNLQESLRSKE | |
| IFNA2b_ | 683 | 2.02 | CDLPQTHSLGSRRTLMLLAQMRRISLFSCLKDRHDFGF | 130 |
| I139Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SYLAVRKYFQRITLYLKEKKYSPCAWEVVRAEIMRSFS | |
| | | | LSTNLQESLRSKE | |
| IFNA2b_ | 678 | 2.64 | CDLPQTHSLGSRRTLMLLAQMRRISLFSCLKDRHDFGF | 131 |
| K187R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSRE | |
| IFNA2b_ | 661 | 2.03 | CDLPQTHSLGSRRTLMLLAQMRRISLFSCLKDRHDFGF | 132 |
| M82K | | | PQEEFGNQFQKAETIPVLHEKIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 654 | 2.18 | CDLPQTHSLGSRRTLMLLAQMRRISLFSCLKDRHDFGF | 133 |
| S177D | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LDTNLQESLRSKE | |
| IFNA2b_ | 653 | 2.13 | CDLPQTHSLGSRRTLMLLAQMRRISLFSCLKDRHDFGF | 134 |
| M82R | | | PQEEFGNQFQKAETIPVLHERIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 641 | 2.02 | CDLPQTHSLGSRRTLMLLAPMRRISLFSCLKDRHDFGF | 135 |
| Q43P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 631 | 2.31 | CDLPQTHSLGSRRTLMLLAQMRRISLQSCLKDRHDFGF | 136 |
| F50Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 572 | 2.62 | CDLPQTHSLGSRRTLMLLAQMRNISLFSCLKDRHDFGF | 137 |
| R46N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 556 | 2.21 | CDLPQTHSLGSRRTYMLLAQMRRISLFSCLKDRHDFGF | 138 |
| L38Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 549 | 2.35 | CDLPQTHSLGSRRTLMLLAQMRRISLSSCLKDRHDFGF | 139 |
| F50S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 548 | 2.26 | CDLPQTHSLGSRRTGMLLAQMRRISLFSCLKDRHDFGF | 140 |
| L38G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 518 | 2.08 | CDLPQTHSLGSRRTLMLLAQMRRISLFSCLKDRHDFGF | 141 |
| R172L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMLSFS | |
| | | | LSTNLQESLRSKE | |
| IFNA2b_ | 497 | 2.24 | CDLPQTHSLGSRRTLMLLAQMRRISLFSCLKDRHDFGF | 142 |
| Q63E | | | PEEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 495 | 2.29 | CDLPQTHSLGSRRTLMLLAQMRRISFFSCLKDRHDFGF | 143 |
| L49F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 483 | 2.15 | CDLPQTHSLGSRRTLMLLAQMRRISLFSCLKDRHDFGF | 144 |
| G125V | | | PQEEFGNQFQKAETIPVLHEMIQQIFNLFSTKDSSAAW | |
| | | | DETLLDKFYTELYQQLNDLEACVIQVVGVTETPLMKED | |
| | | | SILAVRKYFQRITLYLKEKKYSPCAWEVVRAEIMRSFS | |
| | | | LSTNLQESLRSKE | |
| IFNA2b_ | 447 | 2.15 | CDLPQTHSLGSRRTLMLLAQMRRISLFSCLKDRHDFGF | 145 |
| R172M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMMSFS | |
| | | | LSTNLQESLRSKE | |
| IFNA2b_ | 446 | 2.26 | CDLPQTHSLGSRRTLMLLAQMRRISLFSCLKDRHDFGF | 146 |
| L176Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | QSTNLQESLRSKE | |
| IFNA2b_ | 442 | 2.04 | CDLPQTHSLGSRRTLMLLAQMRRISLFSCLKDRHDFGF | 147 |
| S175V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFV | |
| | | | LSTNLQESLRSKE | |
| IFNA2b_ | 425 | 2.28 | CDLPQTHSLGSRRTPMLLAQMRRISLFSCLKDRHDFGF | 148 |
| L38P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 414 | 2.43 | CDLPQTHSLGSRRTLMLLAQMRRISLFSCLKDRHDFGF | 149 |
| P160E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSECAWEVVRAEIMRSFS | |
| | | | LSTNLQESLRSKE | |
| IFNA2b_ | 371 | 2.17 | CDLPQTHSLGSRRTLMLLAQMRSISLFSCLKDRHDFGF | 150 |
| 1 | | | PQEEFGNQFQKAETIPVLHEMIQQIFNLFSTKDSSAAW | |
| R46S | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 368 | 2.15 | CDLPQTHSLGSRRTLMLLAQMRRISLFSCLKDRHDFGF | 151 |
| S173N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRNES | |
| | | | LSTNLQESLRSKE | |
| IFNA2b_ | 359 | 2.01 | CDLPQTHSLGSRRTLMRLAQMRRISLFSCLKDRHDFGF | 152 |
| L40R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 343 | 2.24 | CDLPQTHSLGSRRTLMLLAQMRRISLFSCLKDRHDFGF | 153 |
| R148Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQYITLYLKEKKYSPCAWEVVRAEIMRSFS | |
| | | | LSTNLQESLRSKE | |
| IFNA2b_ | 328 | 2.13 | CDLPQTHSLGSRRTLMLLAQMRRIYLFSCLKDRHDFGF | 154 |
| S48Y | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 318 | 2.09 | CDLPQTHSLGSRRTLMLLAQMRRISLMSCLKDRHDFGF | 155 |
| F50M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 302 | 2.08 | CDLPQTHSLGSRRTLMLLAQMRIISLFSCLKDRHDFGF | 156 |
| R46I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 302 | 2.25 | CDLPQTHSLGSRRTLMLLAQMRRISLFSCLKDRHDFGF | 157 |
| K106L | | | PQEEFGNQFQKAETIPVLHEMIQQIFNLFSTKDSSAAW | |
| | | | DETLLDLFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 301 | 2.13 | CDLPQTHDLGSRRTLMLLAQMRRISLFSCLKDRHDFGF | 158 |
| S31D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 299 | 2.11 | CDLPQTHSLGSRRTLMLLAQMRRISLFSCLKDRHDFGG | 159 |
| F61G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 293 | 2.31 | CDLPQTHSLGSRRTLMLLAQMRRISLFSCLKDRHDFGF | 160 |
| I139F | | | PQEEFGNQFQKAETIPVLHEMIQQIFNLFSTKDSSAAW | |
| | | | DETLLDKFYTELYQQLNDLEACVIQGVGVTETPLMKED | |
| | | | SFLAVRKYFQRITLYLKEKKYSPCAWEVVRAEIMRSFS | |
| | | | LSTNLQESLRSKE | |
| IFNA2b_ | 292 | 2.23 | CDLPQTHSLGSRRTLMLLAQMRRISLFSCLKDRHDFGF | 161 |
| R185D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLDSKE | |
| IFNA2b_ | 292 | 2.19 | CDLPQTHSLGSRRTLMLLAQMRGISLFSCLKDRHDFGF | 162 |
| R46G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 286 | 2.52 | CDLPQTHSLGSRRTLMLLAQMRRISLFSCLKDRHDFGF | 163 |
| Q147S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFSRITLYLKEKKYSPCAWEVVRAEIMRSFS | |
| | | | LSTNLQESLRSKE | |
| IFNA2b_ | 284 | 2.15 | CDLPQTHSLGSRRTLMLLAQMPRISLFSCLKDRHDFGF | 164 |
| R45P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 284 | 2.13 | CDLPQTHSLGSRRTLMLLAQMRRISLFSCLKDRHDFGF | 165 |
| M82G | | | PQEEFGNQFQKAETIPVLHEGIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 265 | 2.34 | CDLPQTHSLGSRRTLMLLAQMRRISGFSCLKDRHDFGF | 166 |
| L49G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 256 | 2.45 | CDLPQTHSLGSRRTLMLLAQMRRISLFSCLKDRHDFGF | 167 |
| L176E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | ESTNLQESLRSKE | |
| IFNA2b_ | 251 | 2.44 | CDLPQTHSLGSRRTLMLLAQMRRISLFSCLKDRHDFGF | 168 |
| K187L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSLE | |
| IFNA2b_ | 250 | 2.29 | CDLPQTHSLGSRRTLMGLAQMRRISLFSCLKDRHDFGF | 169 |
| L40G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 247 | 2.50 | CDLPQTHSLGSRRTLMLLAQMRRISLFSCLKDRHDFGF | 170 |
| R172K | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMKSES | |
| | | | LSTNLQESLRSKE | |
| IFNA2b_ | 240 | 2.04 | CDLPQTHSLGSRRTLMLLAQMRRISLFSCLKDRHDFGF | 171 |
| V78E | | | PQEEFGNQFQKAETIPELHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 239 | 2.11 | CDLPQTHSLGSRRTLMLLAQMRRISLFSCLKDRHDFGI | 172 |
| F61I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 239 | 2.09 | CDLPQTHSLGSRRTLMLLAQMRRISLFSCLKDRHDFGF | 173 |
| L176V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | VSTNLQESLRSKE | |
| IFNA2b_ | 239 | 2.17 | CDLPQTHSLGSRRTLMLLAQMRRISLFSCLKDRHDFGF | 174 |
| E169S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRASIMRSES | |
| | | | LSTNLQESLRSKE | |
| IFNA2b_ | 237 | 2.27 | CDLPQTHSLGSRRTLMLLAQMRRISLFSCLLDRHDFGF | 175 |
| K54L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 236 | 2.07 | CDLPQTHSLGSRRTLMLLAQMRRISLFSCLKDRHDFGF | 176 |
| E169G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAGIMRSFS | |
| | | | LSTNLQESLRSKE | |
| IFNA2b_ | 236 | 2.10 | CDLPQTHSLGSRRTLMLLAQMRRISLFSCLKDRHDFGV | 177 |
| F61V | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 231 | 2.10 | CDLPQTHSLGSRRTLMLLAQMRRISLFSCLKDRHDFGF | 178 |
| S173W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRWFS | |
| | | | LSTNLQESLRSKE | |
| IFNA2b_ | 228 | 2.23 | CDLPQTHSLGSRRTLMLLAQMRRISLFSCLKDRHDFGP | 179 |
| F61P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 218 | 2.23 | CDLPQTHSLGSRRTLMLLAQMRRISLFSCLKDRHDFGF | 180 |
| S186L | | | PQEEFGNQFQKAETIPVLHEMIQQIENLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRLKE | |
| IFNA2b_ | 216 | 2.46 | CDLPQTHSLGSRRTLMLLAQMRRISLFSCLKDRHDFGF | 181 |
| K187E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSEE | |
| IFNA2b_ | 214 | 2.38 | CDLPQTHSLGSRRTLMLLAQMRRISLFSCLKDRHDFGF | 182 |
| K187Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSYE | |
| IFNA2b_ | 211 | 2.10 | CDLPQTHSLGSRRTLMLLAQMRRISLFSCLKDRHDFGF | 183 |
| S175W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFW | |
| | | | LSTNLQESLRSKE | |
| IFNA2b_ | 209 | 2.39 | CDLPQTHSLGSRRTSMLLAQMRRISLFSCLKDRHDFGF | 184 |
| L38S | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 209 | 2.33 | CDLPQTHSLGSRRTLMLLAQMRRISEFSCLKDRHDFGF | 185 |
| L49E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 208 | 2.29 | CDLPQTHSLGSRRTLMLLAQMRRISLFSCLKDRHDFGQ | 186 |
| F61Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 207 | 2.35 | CDLPQTHSLGSRRTLMLLAQMRRISLFSCLKDRHDFGF | 187 |
| Q113W | | | PQEEFGNQFQKAETIPVLHEMIQQIFNLFSTKDSSAAW | |
| | | | DETLLDKFYTELYWQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 206 | 2.15 | CDLPQTHSLGSRRTLMLLAQMRRISLFSCLIDRHDFGF | 188 |
| K54I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 204 | 2.24 | CDLPQTHSLGSRRTLMLLAQMRRISLFSCLKDRHDFGF | 189 |
| K156D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEDKYSPCAWEVVRAEIMRSFS | |
| | | | LSTNLQESLRSKE | |
| IFNA2b_ | 203 | 2.03 | CDLPQTHSLGSRRTLMLLAQMRRISLFSCLKDRHDFGF | 190 |
| P132W | | | PQEEFGNQFQKAETIPVLHEMIQQIFNLFSTKDSSAAW | |
| | | | DETLLDKFYTELYQQLNDLEACVIQGVGVTETWLMKED | |
| | | | SILAVRKYFQRITLYLKEKKYSPCAWEVVRAEIMRSFS | |
| | | | LSTNLQESLRSKE | |
| IFNA2b_ | 202 | 2.07 | CDLPQTHSLGSRRTLMLLAQMRRISLFDCLKDRHDFGF | 191 |
| S51D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 202 | 2.11 | CDLPQTHSLGSRRTLMLLAQQRRISLFSCLKDRHDFGF | 192 |
| M44Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 200 | 2.03 | CDLPQTHSLGSRRTLMLLAQMRRISLFSCLKDRHDFGF | 193 |
| E119Y | | | PQEEFGNQFQKAETIPVLHEMIQQIFNLFSTKDSSAAW | |
| | | | DETLLDKFYTELYQQLNDLYACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 200 | 2.50 | CDLPQTHSLGSRRTLMLLAQMRRISLFSCLKDRHDFGF | 194 |
| A168D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRDEIMRSFS | |
| | | | LSTNLQESLRSKE | |
| IFNA2b_ | 199 | 2.26 | CDLPQTHSLGSRRTLMLLAQMRRISLHSCLKDRHDFGF | 195 |
| F50H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 198 | 2.03 | CDLPQTHSLGSRRTLMLLAQMRRISLFECLKDRHDFGF | 196 |
| S51E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 197 | 2.51 | CDLPQTHSLGSRRTLMLLAQMRRISLFSCLKDRHDFGF | 197 |
| S183F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQEFLRSKE | |
| IFNA2b_ | 194 | 2.05 | CDLPQTHSLGSRRTLMLLAQMRRISLFSCLKDRHDFGF | 198 |
| P160F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSFCAWEVVRAEIMRSFS | |
| | | | LSTNLQESLRSKE | |
| IFNA2b_ | 192 | 2.27 | CDLPQTHSLGSRRTLMLLAQMRRISLFSCLKDRHDFGF | 199 |
| 1 | | | PQEEFGNQFQKAETIPVLHEMIQQIFNLFSTKDSSAAW | |
| E110P | | | DETLLDKFYTPLYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 190 | 2.10 | CDLPQTHSLGSRRTLMLLAQMRRISLFSCLKDRHDFGF | 200 |
| I170L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAELMRSFS | |
| | | | LSTNLQESLRSKE | |
| IFNA2b_ | 189 | 2.10 | CDLPQTHSLGSRRTLMLLAQMRRISLFSCLKDRHDFGF | 201 |
| S175Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFY | |
| | | | LSTNLQESLRSKE | |
| IFNA2b_ | 189 | 2.32 | CDLPQTHSLGSRRTLMLLAQMRRISLFSCLKDRHDFGF | 202 |
| S175G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFG | |
| | | | LSTNLQESLRSKE | |
| IFNA2b_ | 185 | 2.00 | CDLPQTHSLGSRRTLMQLAQMRRISLFSCLKDRHDFGF | 203 |
| L40Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 183 | 2.31 | CDLPQTHSLGSRRTLMLLAQMRRISLFSCLKDRHDFGF | 204 |
| K187V | | | PQEEFGNQFQKAETIPVLHEMIQQIENLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSVE | |
| IFNA2b_ | 180 | 2.16 | CDLPQTHSLGSRRTLMLLAQMRRISLFSCLKDRHDFGF | 205 |
| K157Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKYYSPCAWEVVRAEIMRSFS | |
| | | | LSTNLQESLRSKE | |
| IFNA2b_ | 180 | 2.02 | CDLPQTHSLGSRRTLMLLAQMRRISLFSCLKDRHDFGF | 206 |
| N116M | | | PQEEFGNQFQKAETIPVLHEMIQQIFNLFSTKDSSAAW | |
| | | | DETLLDKFYTELYQQLMDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 180 | 2.11 | CDLPQTHSLGSRRTLMLLAQMRRISLFSCLKDRHDFGF | 207 |
| I86L | | | PQEEFGNQFQKAETIPVLHEMIQQLFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 177 | 2.20 | CDLPQTHSLGSRRTLMLLAQMRRISLFSCLKDRHDFGF | 208 |
| P62I | | | IQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 175 | 2.00 | CDLPQTHSLGSRRTLMLLAQMRRISLFSCLKDRHDFGF | 209 |
| Y145H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKHFQRITLYLKEKKYSPCAWEVVRAEIMRSES | |
| | | | LSTNLQESLRSKE | |
| IFNA2b_ | 167 | 2.09 | CDLPQTHSLGSRRTLMLLAQMRRISLFSCLKDRHDFGF | 210 |
| V126Y | | | PQEEFGNQFQKAETIPVLHEMIQQIFNLFSTKDSSAAW | |
| | | | DETLLDKFYTELYQQLNDLEACVIQGYGVTETPLMKED | |
| | | | SILAVRKYFQRITLYLKEKKYSPCAWEVVRAEIMRSFS | |
| | | | LSTNLQESLRSKE | |
| IFNA2b_ | 166 | 2.17 | CDLPQTHSLGSRRTLMLLAQMRRISLFSCLKDRHDFGF | 211 |
| Q69S | | | PQEEFGNSFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 162 | 2.52 | CDLPQTHSLGSRRTLMLLAQMRRISLFSCLKDRHDFGF | 212 |
| K187M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSME | |
| IFNA2b_ | 160 | 2.11 | CDLPQTHSLGSRRTLMLLAQMRRISLFSCLKDRHDFGF | 213 |
| N116F | | | PQEEFGNQFQKAETIPVLHEMIQQIFNLFSTKDSSAAW | |
| | | | DETLLDKFYTELYQQLFDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 159 | 2.24 | CDLPQTHSLGSRRTLMLLAQMRRISLFSCLKDRHDFGF | 214 |
| P160W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSWCAWEVVRAEIMRSFS | |
| | | | LSTNLQESLRSKE | |
| IFNA2b_ | 158 | 2.51 | CDLPQTHSLGSRRTLMLLAQMRRISLFSCLKDRHDFGF | 215 |
| K187F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSFE | |
| IFNA2b_ | 158 | 2.32 | CDLPQTHSLGSRRTLMLLAQMRRISLFSCLKDRHDFGF | 216 |
| K187I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSIE | |
| IFNA2b_ | 158 | 2.10 | CDLPQTHSLGSRRTTMLLAQMRRISLESCLKDRHDFGF | 217 |
| L38T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 157 | 2.06 | CDLPQTHSLGSRRTLMLLAQMRRISLFSCLKDRHDFGF | 218 |
| S183M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQEMLRSKE | |
| IFNA2b_ | 153 | 2.09 | CDLPQTHSLGSRRTLMLLAQMRRISLFSCLKDRHDFGF | 219 |
| V165D | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEDVRAEIMRSFS | |
| | | | LSTNLQESLRSKE | |
| IFNA2b_ | 153 | 2.19 | CDLPQTHSLGSRRTLMLLGQMRRISLFSCLKDRHDFGF | 220 |
| A42G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 152 | 2.00 | CDLPQTHSLGSRRTLMLLAQMRRISLFSCLKDRHDFGF | 221 |
| K156L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKELKYSPCAWEVVRAEIMRSFS | |
| | | | LSTNLQESLRSKE | |
| IFNA2b_ | 149 | 2.37 | CDLPQTHSLGSRRTLMLLAQMRRISLFSCLKDRHDFGF | 222 |
| S183E | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQEELRSKE | |
| IFNA2b_ | 149 | 2.19 | CDLPQTHSLGSRRTLMLLAQMRRISLFSCLKDRHDFGF | 223 |
| 1 | | | PQEEFGNQFQKAETIPVLHEMIQQIFNLFSTKDSSAAW | |
| Y108K | | | DETLLDKFKTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 149 | 2.20 | CDLPQTHSLGSRRTLMLLAQMRRISLFSCLKNRHDFGF | 224 |
| D55N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 149 | 2.25 | CDLPQTHSLGSRRTQMLLAQMRRISLFSCLKDRHDFGF | 225 |
| L38Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 147 | 2.32 | CDLPQTHSLGSRRTLMLLAQMRRISLFSCLKDRHDFGF | 226 |
| R185E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLESKE | |
| IFNA2b_ | 146 | 2.08 | CDLPQTHSLGSRRTLMLLAQMRRISLFSCLKDRHDFGF | 227 |
| S186I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRIKE | |
| IFNA2b_ | 146 | 2.05 | CDLPQTHSLGSRRTLMLLAQMRRISLFSCLKDRHDFGF | 228 |
| S175E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFE | |
| | | | LSTNLQESLRSKE | |
| IFNA2b_ | 146 | 2.38 | CDLPQTHSLGSRRTLMLLAQMRRISPFSCLKDRHDFGF | 229 |
| L49P | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 146 | 2.37 | CDLPQTHSLGSRRTLMLLAQMRRISLFSCLKDRHDFGF | 230 |
| S183Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQEYLRSKE | |
| IFNA2b_ | 144 | 2.27 | CDLPQTHSLGSRRTLMLLAQMRAISLFSCLKDRHDFGF | 231 |
| R46A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 144 | 2.04 | CDLPQTASLGSRRTLMLLAQMRRISLESCLKDRHDFGF | 232 |
| H30A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 143 | 2.38 | CDLPQTHSLGSRRTLMLLAQMRHISLFSCLKDRHDFGF | 233 |
| R46H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 142 | 2.34 | CDLPQTHSLGSRRTLMLLAQMRRISLFSCLKDRHDFGF | 234 |
| E110S | | | PQEEFGNQFQKAETIPVLHEMIQQIFNLFSTKDSSAAW | |
| | | | DETLLDKFYTSLYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 141 | 2.10 | CDLPQTHSLGSRRTLMLLAQMRRISLFSCLKDRHDFGF | 235 |
| K156W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEWKYSPCAWEVVRAEIMRSES | |
| | | | LSTNLQESLRSKE | |
| IFNA2b_ | 140 | 2.53 | CDLPQTHSLGSRRTLMLLAQMRTISLFSCLKDRHDFGF | 236 |
| R46T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 139 | 2.36 | CDLPQTHSLGSRRTLMLLAQMRRISLFSCLKDRHDFGF | 237 |
| S175Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFQ | |
| | | | LSTNLQESLRSKE | |
| IFNA2b_ | 138 | 2.15 | CDLPQTHSLGSRRTLMLLAQMRRISLFSCLKDRHQFGF | 238 |
| D58Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 137 | 2.21 | CDLPQTHSLGSRRTLMLLAQMRRISLFSCLKDRHDFGF | 239 |
| E155H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKHKKYSPCAWEVVRAEIMRSFS | |
| | | | LSTNLQESLRSKE | |
| IFNA2b_ | 136 | 2.12 | CDLPQTHSLGSRRTLMDLAQMRRISLFSCLKDRHDFGF | 240 |
| L40D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 136 | 2.02 | CDLPQTHSLGSRRTLMLLAQMRRISLFSCLKDRHDFGF | 241 |
| I | | | PQEEFGNQFQKAETIPVLHEMIQQIFNLESTKDSSAAW | |
| A120M | | | DETLLDKFYTELYQQLNDLEMCVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 135 | 2.02 | CDLPQTHSLGSRRTLMLLAQMRRISLFSCLKDRHDFGF | 242 |
| R167H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVHAEIMRSFS | |
| | | | LSTNLQESLRSKE | |
| IFNA2b_ | 134 | 2.11 | CDLPQTHSLGSRRTLMLLAQMRRISLFSCLKDRHDFGF | 243 |
| M82S | | | PQEEFGNQFQKAETIPVLHESIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 132 | 2.27 | CDLPQTHSLGSRRTLMLLMQMRRISLFSCLKDRHDFGF | 244 |
| A42M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 131 | 2.10 | CDLPQTHSLGSRRTLMLLAQMRRIHLFSCLKDRHDFGF | 245 |
| S48H | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 129 | 2.02 | CDLPQTHSLGSRRTLMLLAQMRRISLFSCLKDRHDFGF | 246 |
| R143F | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVFKYFQRITLYLKEKKYSPCAWEVVRAEIMRSES | |
| | | | LSTNLQESLRSKE | |
| IFNA2b_ | 129 | 2.49 | CDLPQTHSLGSRRTLMLLAQMRRISLFSCLKDRHDFGF | 247 |
| K187W | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSWE | |
| IFNA2b_ | 128 | 2.01 | CDLPQTHSLGSRRTLMLLAQMRRSSLFSCLKDRHDFGF | 248 |
| I47S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 128 | 2.43 | CDLPQTHSLGSNRTLMLLAQMRRISLFSCLKDRHDFGF | 249 |
| R35N | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 128 | 2.13 | CDLPQTHSLGSRRTLMLLAQMRRISLFSCLKDRHDFGF | 250 |
| S175T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFT | |
| | | | LSTNLQESLRSKE | |
| IFNA2b_ | 127 | 2.10 | CDLPQTHSLGSRRTLMLLAQMRRISLFSCLKDRHDFGF | 251 |
| S173Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRYFS | |
| | | | LSTNLQESLRSKE | |
| IFNA2b_ | 125 | 2.37 | CDLPQTHSLGSRRTLMLLAQMRRISLFSCLKDRHDFGF | 252 |
| A168M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRMEIMRSFS | |
| | | | LSTNLQESLRSKE | |
| IFNA2b_ | 125 | 2.05 | CDLPQTHSLGSRRTLMLLAQMRRISLFSCLKDRHDFGF | 253 |
| A98E | | | PQEEFGNQFQKAETIPVLHEMIQQIFNLFSTKDSSAEW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 125 | 2.07 | CDLPQTHSLGSRRTLMLLAQMRRISLFSCLKDRHDFGF | 254 |
| S186D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRDKE | |
| IFNA2b_ | 124 | 2.31 | CDLPQTHSLGSRRTLMLLAQMRRISLFSCLKDRHDFGF | 255 |
| M134Y | | | PQEEFGNQFQKAETIPVLHEMIQQIFNLFSTKDSSAAW | |
| | | | DETLLDKFYTELYQQLNDLEACVIQGVGVTETPLYKED | |
| | | | SILAVRKYFQRITLYLKEKKYSPCAWEVVRAEIMRSES | |
| | | | LSTNLQESLRSKE | |
| IFNA2b_ | 124 | 2.12 | CDLPQTDSLGSRRTLMLLAQMRRISLFSCLKDRHDFGF | 256 |
| H30D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 122 | 2.08 | CDLPQTHSLGSRRTLMLLAQMRRISLFSCLKDRHDFGF | 257 |
| E119F | | | PQEEFGNQFQKAETIPVLHEMIQQIFNLFSTKDSSAAW | |
| | | | DETLLDKFYTELYQQLNDLFACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 121 | 2.09 | CDLPQTHSLGSRRTLMLLAQMRRISLESCLKDRHDFGF | 258 |
| V78G | | | PQEEFGNQFQKAETIPGLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 120 | 2.22 | CDLPQTHSLGSRRTLMLLAQMRRISLFSCLKDRHDFGF | 259 |
| R185Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLQSKE | |
| IFNA2b_ | 119 | 2.09 | CDLPQTHSLGSRRTLMLLAQMRRISLFSCLMDRHDFGF | 260 |
| K54M | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 119 | 2.17 | CDLPQTHSDGSRRTLMLLAQMRRISLFSCLKDRHDFGF | 261 |
| L32D | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 117 | 2.02 | CDLPQTHSLGSRRTLMLLAQMRRISLFSCLKDRHDFGF | 262 |
| L103E | | | PQEEFGNQFQKAETIPVLHEMIQQIFNLFSTKDSSAAW | |
| | | | DETELDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 116 | 2.11 | CDLPQTHSLGSRRTLMLLAQMRRISLFSCLKDRHDFGF | 263 |
| K106D | | | PQEEFGNQFQKAETIPVLHEMIQQIFNLFSTKDSSAAW | |
| | | | DETLLDDFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 114 | 2.13 | CDLPQTHSLGSRRTNMLLAQMRRISLFSCLKDRHDFGF | 264 |
| L38N | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 114 | 2.31 | CDLPQTHSLGSRRTLMLLAQMRRISLFSCLKDRHDFGF | 265 |
| N179G | | | PQEEFGNQFQKAETIPVLHEMIQQIENLESTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTGLQESLRSKE | |
| IFNA2b_ | 111 | 2.21 | CDLPQTHSLGSRRTLMLLAQMRRISLFSCLKDRHDFGF | 266 |
| V165I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEIVRAEIMRSFS | |
| | | | LSTNLQESLRSKE | |
| IFNA2b_ | 111 | 2.25 | CDLPQTHSLGSRRTLMLLAQMRRISLFSCLKDRHDFGF | 267 |
| L103D | | | PQEEFGNQFQKAETIPVLHEMIQQIFNLESTKDSSAAW | |
| | | | DETDLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 110 | 2.14 | CDLPQTHSLGSRRTLMLLAQMRRISLFSCLKDRHDFGF | 268 |
| K187G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSGE | |
| IFNA2b_ | 110 | 2.01 | CDLPQTHSLGSRGTLMLLAQMRRISLFSCLKDRHDFGF | 269 |
| R36G | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 110 | 2.22 | CDLPQTHSLGSRRTLMLLAQMRRISLFSCLKDRHDFGF | 270 |
| A141E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILEVRKYFQRITLYLKEKKYSPCAWEVVRAEIMRSES | |
| | | | LSTNLQESLRSKE | |
| IFNA2b_ | 110 | 2.28 | CDLPQTHSLGSRRTLMLLAQMRRISLFSCLKDRHDFGF | 271 |
| T129G | | | PQEEFGNQFQKAETIPVLHEMIQQIFNLFSTKDSSAAW | |
| | | | DETLLDKFYTELYQQLNDLEACVIQGVGVGETPLMKED | |
| | | | SILAVRKYFQRITLYLKEKKYSPCAWEVVRAEIMRSFS | |
| | | | LSTNLQESLRSKE | |
| IFNA2b_ | 109 | 2.27 | CDLPQTHSLGSRRTLMLLAQMRRISLFSCLKDRHDFGF | 272 |
| A168Q | | | PQEEFGNQFQKAETIPVLHEMIQQIFNLESTKDSSAAW | |
| | | | DETLLDKFYTELYQQLNDLEACVIQGVGVTETPLMKED | |
| | | | SILAVRKYFQRITLYLKEKKYSPCAWEVVRQEIMRSFS | |
| | | | LSTNLQESLRSKE | |
| IFNA2b_ | 109 | 2.09 | CDLPQTHSLGSRRTLMLLAQMRRISLFSCLKQRHDFGF | 273 |
| D55Q | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 109 | 2.02 | CDLPQTHSLGSRRTLMLLAQMRRISLESCLKDRHDFGF | 274 |
| S183K | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQEKLRSKE | |
| IFNA2b_ | 109 | 2.51 | CDLPQTHSLGSRRTLMLLAQMRRISLFSCLKDRHDFGF | 275 |
| S183L | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQELLRSKE | |
| IFNA2b_ | 108 | 2.08 | CDLPQTHSLGSRRTLMLLAQMRRISLFSCLKDRHDFGF | 276 |
| N116Q | | | PQEEFGNQFQKAETIPVLHEMIQQIFNLFSTKDSSAAW | |
| | | | DETLLDKFYTELYQQLQDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 108 | 2.14 | CDLPQTHSLGSRRTLMLLAQMRRISLFSCLKDRHDFGF | 277 |
| S177R | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LRTNLQESLRSKE | |
| IFNA2b_ | 108 | 2.13 | CDLPQTHSLGSRRTLMLLAQMRRISLFSCLKDRHDFGF | 278 |
| K93E | | | PQEEFGNQFQKAETIPVLHEMIQQIFNLFSTEDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 107 | 2.27 | CDLPQTHSLGSRRTLMLLAQMRRISLFSCLKDRHDFGF | 279 |
| A141I | | | PQEEFGNQFQKAETIPVLHEMIQQIFNLFSTKDSSAAW | |
| | | | DETLLDKFYTELYQQLNDLEACVIQGVGVTETPLMKED | |
| | | | SILIVRKYFQRITLYLKEKKYSPCAWEVVRAEIMRSFS | |
| | | | LSTNLQESLRSKE | |
| IFNA2b_ | 107 | 2.29 | CDLPQTHSLGSRRTLMLLAQMRRISLFSCLKDRHDFGF | 280 |
| T109W | | | PQEEFGNQFQKAETIPVLHEMIQQIFNLFSTKDSSAAW | |
| | | | DETLLDKFYWELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 106 | 2.20 | CDLPQTHSLGSRRTLMLLAQMRRISLFSCLKDRHDFGF | 281 |
| K72D | | | PQEEFGNQFQDAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 105 | 2.33 | CDLPQTHSLGSRRTLMLLAQMRRISLASCLKDRHDFGF | 282 |
| F50A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 105 | 2.20 | CDLPQTHSLGSRRTLMLLAQMRRISLFSCLKDRHDFGA | 283 |
| F61A | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSES | |
| | | | LSTNLQESLRSKE | |
| IFNA2b_ | 104 | 2.27 | CDLPQTHSLGSRRTLMLLAQMRRISLFSCLKDRHDFGS | 284 |
| F61S | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 103 | 2.27 | CDLPQTHSLGSRRTLMLLAQMRRISLFSCLKDRHDFGF | 285 |
| P160Y | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSYCAWEVVRAEIMRSES | |
| | | | LSTNLQESLRSKE | |
| IFNA2b_ | 103 | 2.04 | CDLPQTHSEGSRRTLMLLAQMRRISLFSCLKDRHDFGF | 286 |
| L32E | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 103 | 2.18 | CDLPQTHSLGSRRTLMLLAQMRRISLFSCLKDRHDFGF | 287 |
| K93V | | | PQEEFGNQFQKAETIPVLHEMIQQIFNLESTVDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |
| IFNA2b_ | 102 | 2.29 | CDLPQTHSLGSRRTLMLLAQMRRISLFSCLKDRHDFGF | 288 |
| P160T | | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSTCAWEVVRAEIMRSFS | |
| | | | LSTNLQESLRSKE | |
| IFNA2b_ | | 2.05 | CDLPQTHSLGSRRTLMLLAQMRRESLFSCLKDRHDFGF | 289 |
| I47E | 102 | | PQEEFGNQFQKAETIPVLHEMIQQIFNLFSTKDSSAAW | |
| | | | DETLLDKFYTELYQQLNDLEACVIQGVGVTETPLMKED | |
| | | | SILAVRKYFQRITLYLKEKKYSPCAWEVVRAEIMRSFS | |
| | | | LSTNLQESLRSKE | |

Table 2 below shows the results for 12 variants that were selected for further analysis. To qualify for prioritization and further characterization first, >50 diploids must have formed between the IFNA2 variant and human IFNAR2 during the AlphaSeq™ assay. In addition, >2 different substitutions at the specific position in IFNA2 must have given rise to detuning. For a given amino acid residue position, the number of substitutions that led to detuning was termed the “multiplicity” for that position. Preference was also given to variants at positions conserved in cynomolgus monkey, mouse, and rat. Using these criteria, 12 initial variants were identified that spanned 4 affinity bins ranging from ˜300 nM (˜10-fold weaker than wild-type) to ˜10 μM (˜300-fold weaker than wild-type).

TABLE 2
Detuned IFNA2 Variants Selected for Further Analysis

| | | IFNAR2 | | |
|---|---|---|---|---|
| Affinity bin | | affinity | Position conserved | |
| (log₁₀nM) | Variant | (log₁₀nM) | in mouse/cyno/rat? | Multiplicity |
| ~4 | I47P | 4.39 | cyno only | 4 |
| | A168G | 4.03 | Yes | 9 |
| | R172T | 3.94 | Yes | 12 |
| ~3.5 | R46F | 3.50 | Yes | 11 |
| | S175K | 3.49 | Yes | 13 |
| | M171L | 3.48 | cyno only | 14 |
| ~3 | S183W | 3.08 | cyno only | 7 |
| | N116W | 3.07 | cyno/mouse | 4 |
| | F50G | 2.94 | cyno only | 8 |
| ~2.5 | M82G | 2.45 | cyno only | 5 |
| | F61G | 2.48 | Yes | 7 |
| | R185D | 2.47 | cyno/rat | 3 |

Table 3 provides affinities between human IFNA2 detuned variants comprising two amino acid substitutions, i.e., double mutants, and human IFNAR2. Any double mutant variant found to bind human IFNAR2 with an affinity weaker than 316 nM (log₁₀ nM=2.5) and found to have an average expression bin>2.0, but that did not show decreased affinity to mouse IFNAR2 was included in Table 3. Table 3 is sorted in order of descending Kd, with the highest values representing the lowest affinity of IFNA2 for human IFNAR2. For example, a variant with the name “IFNA2b_M44N_A168R” has an amino acid substitution in SEQ ID NO: 1 at location 44, where the existing methionine is replaced with asparagine AND a substitution at location 168, where the existing alanine is replaced with arginine. Table 3 also provides the polypeptide sequences of the 127 IFNA2 variants comprising two amino acid substitutions as SEQ ID NOs 290-416. The polypeptide sequences describe the mature IFNA2b protein, which does not include a 23-amino acid signal peptide that is cleaved from the N-terminus to produce the mature protein.

TABLE 3
IFNA2 Double Mutants with Detuned Affinity to Human IFNAR2

| | Affinity | | | |
|---|---|---|---|---|
| | to | | | |
| | human | Average | | |
| | IFNAR2 | expression | | SEQ |
| Variant | (nM) | bin | Polypeptide Sequence | ID NO |
| IFNA2b_M44N_ | 124223 | 2.21 | CDLPQTHSLGSRRTLMLLAQNRRISLFS | 290 |
| A168R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRREIMRSFSLSTNLQESLRSKE | |
| IFNA2b_L49P_ | 37506 | 2.20 | CDLPQTHSLGSRRTLMLLAQMRRISPFS | 291 |
| S175P | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFPLSTNLQESLRSKE | |
| IFNA2b_L49P_ | 36825 | 2.07 | CDLPQTHSLGSRRTLMLLAQMRRISPFS | 292 |
| S173H | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRHFSLSTNLQESLRSKE | |
| IFNA2b_L49P_ | 28512 | 2.26 | CDLPQTHSLGSRRTLMLLAQMRRISPFS | 293 |
| M171W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIWRSFSLSTNLQESLRSKE | |
| IFNA2b_L49P_ | 24545 | 2.26 | CDLPQTHSLGSRRTLMLLAQMRRISPFS | 294 |
| M171G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIGRSFSLSTNLQESLRSKE | |
| IFNA2b_L49P_ | 23380 | 2.24 | CDLPQTHSLGSRRTLMLLAQMRRISPFS | 295 |
| S173R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRRFSLSTNLQESLRSKE | |
| IFNA2b_M44N_ | 21816 | 2.03 | CDLPQTHSLGSRRTLMLLAQNRRISLFS | 296 |
| S175P | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFPLSTNLQESLRSKE | |
| IFNA2b_L49P_ | 21682 | 2.33 | CDLPQTHSLGSRRTLMLLAQMRRISPFS | 297 |
| M171V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIVRSFSLSTNLQESLRSKE | |
| IFNA2b_L49P_ | 17082 | 2.47 | CDLPQTHSLGSRRTLMLLAQMRRISPFS | 298 |
| M171K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIKRSFSLSTNLQESLRSKE | |
| IFNA2b_L49P_ | 16469 | 2.22 | CDLPQTHSLGSRRTLMLLAQMRRISPFS | 299 |
| M171S | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEISRSFSLSTNLQESLRSKE | |
| IFNA2b_L49P_ | 16007 | 2.16 | CDLPQTHSLGSRRTLMLLAQMRRISPFS | 300 |
| S175R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFRLSTNLQESLRSKE | |
| IFNA2b_L49P_ | 14780 | 2.28 | CDLPQTHSLGSRRTLMLLAQMRRISPFS | 301 |
| M171R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIRRSFSLSTNLQESLRSKE | |
| IFNA2b_L49P_ | 13375 | 2.18 | CDLPQTHSLGSRRTLMLLAQMRRISPFS | 302 |
| L176G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSGSTNLQESLRSKE | |
| IFNA2b_L49P_ | 12703 | 2.32 | CDLPQTHSLGSRRTLMLLAQMRRISPFS | 303 |
| M171Y | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIYRSFSLSTNLQESLRSKE | |
| IFNA2b_L49P_ | 11472 | 2.14 | CDLPQTHSLGSRRTLMLLAQMRRISPFS | 304 |
| L176N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSNSTNLQESLRSKE | |
| IFNA2b_L49P_ | 11317 | 2.16 | CDLPQTHSLGSRRTLMLLAQMRRISPFS | 305 |
| S173Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRQFSLSTNLQESLRSKE | |
| IFNA2b_L49P_ | 10571 | 2.01 | CDLPQTHSLGSRRTLMLLAQMRRISPFS | 306 |
| L176H | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSHSTNLQESLRSKE | |
| IFNA2b_L49P_ | 8793 | 2.07 | CDLPQTHSLGSRRTLMLLAQMRRISPFS | 307 |
| L176R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSRSTNLQESLRSKE | |
| IFNA2b_L49P_ | 8575 | 2.22 | CDLPQTHSLGSRRTLMLLAQMRRISPFS | 308 |
| M171A | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIARSFSLSTNLQESLRSKE | |
| IFNA2b_L49P_ | 7719 | 2.35 | CDLPQTHSLGSRRTLMLLAQMRRISPFS | 309 |
| M171T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEITRSFSLSTNLQESLRSKE | |
| IFNA2b_L49P_ | 7334 | 2.21 | CDLPQTHSLGSRRTLMLLAQMRRISPFS | 310 |
| L176A | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSASTNLQESLRSKE | |
| IFNA2b_L49P_ | 6922 | 2.18 | CDLPQTHSLGSRRTLMLLAQMRRISPFS | 311 |
| M171F | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIFRSFSLSTNLQESLRSKE | |
| IFNA2b_M44N_ | 4872 | 2.04 | CDLPQTHSLGSRRTLMLLAQNRRISLFS | 312 |
| S175K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFKLSTNLQESLRSKE | |
| IFNA2b_L49P_ | 4674 | 2.39 | CDLPQTHSLGSRRTLMLLAQMRRISPFS | 313 |
| S175K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFKLSTNLQESLRSKE | |
| IFNA2b_L49P_ | 4409 | 2.34 | CDLPQTHSLGSRRTLMLLAQMRRISPFS | 314 |
| L176E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSESTNLQESLRSKE | |
| IFNA2b_L49P_ | 3823 | 2.06 | CDLPQTHSLGSRRTLMLLAQMRRISPFS | 315 |
| E169T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRATIMRSFSLSTNLQESLRSKE | |
| IFNA2b_L49P_ | 2447 | 2.06 | CDLPQTHSLGSRRTLMLLAQMRRISPFS | 316 |
| M171L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEILRSFSLSTNLQESLRSKE | |
| IFNA2b_L49P_ | 2222 | 2.20 | CDLPQTHSLGSRRTLMLLAQMRRISPFS | 317 |
| L176Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSQSTNLQESLRSKE | |
| IFNA2b_L49P_ | 2093 | 2.16 | CDLPQTHSLGSRRTLMLLAQMRRISPFS | 318 |
| K187E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSEE | |
| IFNA2b_L49P_ | 1993 | 2.28 | CDLPQTHSLGSRRTLMLLAQMRRISPFS | 319 |
| S173N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRNFSLSTNLQESLRSKE | |
| IFNA2b_M44N_ | 1933 | 2.00 | CDLPQTHSLGSRRTLMLLAQNRRISLFS | 320 |
| L176H | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSHSTNLQESLRSKE | |
| IFNA2b_M44N_ | 1912 | 2.01 | CDLPQTHSLGSRRTLMLLAQNRRISLFS | 321 |
| S175R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFRLSTNLQESLRSKE | |
| IFNA2b_L49P_ | 1871 | 2.10 | CDLPQTHSLGSRRTLMLLAQMRRISPFS | 322 |
| S177N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLNTNLQESLRSKE | |
| IFNA2b_L49P_ | 1702 | 2.23 | CDLPQTHSLGSRRTLMLLAQMRRISPFS | 323 |
| S173W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRWFSLSTNLQESLRSKE | |
| IFNA2b_L49P_ | 1664 | 2.32 | CDLPQTHSLGSRRTLMLLAQMRRISPFS | 324 |
| S177D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLDTNLQESLRSKE | |
| IFNA2b_L49P_ | 1628 | 2.27 | CDLPQTHSLGSRRTLMLLAQMRRISPFS | 325 |
| R185D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLDSKE | |
| IFNA2b_L49P_ | 1590 | 2.06 | CDLPQTHSLGSRRTLMLLAQMRRISPFS | 326 |
| E169S | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRASIMRSFSLSTNLQESLRSKE | |
| IFNA2b_L49P_ | 1420 | 2.20 | CDLPQTHSLGSRRTLMLLAQMRRISPFS | 327 |
| S175G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFGLSTNLQESLRSKE | |
| IFNA2b_L49P_ | 1411 | 2.23 | CDLPQTHSLGSRRTLMLLAQMRRISPFS | 328 |
| S177L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLLTNLQESLRSKE | |
| IFNA2b_L49P_ | 1359 | 2.40 | CDLPQTHSLGSRRTLMLLAQMRRISPFS | 329 |
| R172K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMKSFSLSTNLQESLRSKE | |
| IFNA2b_L49P_ | 1325 | 2.44 | CDLPQTHSLGSRRTLMLLAQMRRISPFS | 330 |
| R185E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLESKE | |
| IFNA2b_L49P_ | 1178 | 2.50 | CDLPQTHSLGSRRTLMLLAQMRRISPFS | 331 |
| K187M | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSME | |
| IFNA2b_L49P_ | 1177 | 2.35 | CDLPQTHSLGSRRTLMLLAQMRRISPFS | 332 |
| K187W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSWE | |
| IFNA2b_L49P_ | 1156 | 2.18 | CDLPQTHSLGSRRTLMLLAQMRRISPFS | 333 |
| S159Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYQPCAW | |
| | | | EVVRAEIMRSFSLSTNLQESLRSKE | |
| IFNA2b_M44N_ | 1046 | 2.11 | CDLPQTHSLGSRRTLMLLAQNRRISLFS | 334 |
| L176Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSQSTNLQESLRSKE | |
| IFNA2b_M44N_ | 978 | 2.38 | CDLPQTHSLGSRRTLMLLAQNRRISLFS | 335 |
| R185E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLESKE | |
| IFNA2b_L49P_ | 900 | 2.28 | CDLPQTHSLGSRRTLMLLAQMRRISPFS | 336 |
| S175Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFQLSTNLQESLRSKE | |
| IFNA2b_L49P_ | 821 | 2.23 | CDLPQTHSLGSRRTLMLLAQMRRISPFS | 337 |
| S175M | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFMLSTNLQESLRSKE | |
| IFNA2b_L49P_ | 821 | 2.07 | CDLPQTHSLGSRRTLMLLAQMRRISPFS | 338 |
| E155V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKVKKYSPCAW | |
| | | | EVVRAEIMRSFSLSTNLQESLRSKE | |
| IFNA2b_L49P_ | 795 | 2.41 | CDLPQTHSLGSRRTLMLLAQMRRISPFS | 339 |
| K187D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSDE | |
| IFNA2b_L49P_ | 789 | 2.08 | CDLPQTHSLGSRRTLMLLAQMRRISPFS | 340 |
| S175W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFWLSTNLQESLRSKE | |
| IFNA2b_L49P_ | 788 | 2.34 | CDLPQTHSLGSRRTLMLLAQMRRISPFS | 341 |
| L176V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSVSTNLQESLRSKE | |
| IFNA2b_L49P_ | 784 | 2.15 | CDLPQTHSLGSRRTLMLLAQMRRISPFS | 342 |
| S173L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRLESLSTNLQESLRSKE | |
| IFNA2b_M44N_ | 784 | 2.01 | CDLPQTHSLGSRRTLMLLAQNRRISLFS | 343 |
| S175G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFGLSTNLQESLRSKE | |
| IFNA2b_L49P_ | 735 | 2.29 | CDLPQTHSLGSRRTLMLLAQMRRISPFS | 344 |
| R185G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLGSKE | |
| IFNA2b_L49P_ | 714 | 2.12 | CDLPQTHSLGSRRTLMLLAQMRRISPFS | 345 |
| R143W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVWKYFQRITLYLKEKKYSPCAW | |
| | | | EVVRAEIMRSFSLSTNLQESLRSKE | |
| IFNA2b_L49P_ | 702 | 2.05 | CDLPQTHSLGSRRTLMLLAQMRRISPFS | 346 |
| I149T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRTTLYLKEKKYSPCAW | |
| | | | EVVRAEIMRSFSLSTNLQESLRSKE | |
| IFNA2b_L49P_ | 661 | 2.49 | CDLPQTHSLGSRRTLMLLAQMRRISPFS | 347 |
| R185L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLLSKE | |
| IFNA2b_L49P_ | 637 | 2.28 | CDLPQTHSLGSRRTLMLLAQMRRISPFS | 348 |
| R185S | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLSSKE | |
| IFNA2b_L49P_ | 632 | 2.11 | CDLPQTHSLGSRRTLMLLAQMRRISPFS | 349 |
| S177H | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLHTNLQESLRSKE | |
| IFNA2b_L49P_ | 625 | 2.00 | CDLPQTHSLGSRRTLMLLAQMRRISPFS | 350 |
| K106D | | | CLKDRHDFGFPQEEFGNQFQKAETIPVL | |
| | | | HEMIQQIFNLFSTKDSSAAWDETLLDDF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_M44N_ | 621 | 2.19 | CDLPQTHSLGSRRTLMLLAQNRRISLFS | 351 |
| R185N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLNSKE | |
| IFNA2b_L49P_ | 569 | 2.44 | CDLPQTHSLGSRRTLMLLAQMRRISPFS | 352 |
| K187V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSVE | |
| IFNA2b_L49P_ | 563 | 2.34 | CDLPQTHSLGSRRTLMLLAQMRRISPFS | 353 |
| K106W | | | CLKDRHDFGFPQEEFGNQFQKAETIPVL | |
| | | | HEMIQQIFNLFSTKDSSAAWDETLLDWF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 562 | 2.31 | CDLPQTHSLGSRRTLMLLAQMRRISPFS | 354 |
| K135T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMT | |
| | | | EDSILAVRKYFQRITLYLKEKKYSPCAW | |
| | | | EVVRAEIMRSFSLSTNLQESLRSKE | |
| IFNA2b_M44N_ | 561 | 2.06 | CDLPQTHSLGSRRTLMLLAQNRRISLFS | 355 |
| V128I | | | CLKDRHDFGFPQEEFGNQFQKAETIPVL | |
| | | | HEMIQQIFNLFSTKDSSAAWDETLLDKF | |
| | | | YTELYQQLNDLEACVIQGVGITETPLMK | |
| | | | EDSILAVRKYFQRITLYLKEKKYSPCAW | |
| | | | EVVRAEIMRSFSLSTNLQESLRSKE | |
| IFNA2b_M44N_ | 559 | 2.05 | CDLPQTHSLGSRRTLMLLAQNRRISLFS | 356 |
| R185D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLDSKE | |
| IFNA2b_L49P_ | 543 | 2.15 | CDLPQTHSLGSRRTLMLLAQMRRISPFS | 357 |
| K106P | | | CLKDRHDFGFPQEEFGNQFQKAETIPVL | |
| | | | HEMIQQIFNLFSTKDSSAAWDETLLDPF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 542 | 2.34 | CDLPQTHSLGSRRTLMLLAQMRRISPFS | 358 |
| S183M | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQEMLRSKE | |
| IFNA2b_L49P_ | 540 | 2.29 | CDLPQTHSLGSRRTLMLLAQMRRISPFS | 359 |
| S183D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQEDLRSKE | |
| IFNA2b_L49P_ | 522 | 2.22 | CDLPQTHSLGSRRTLMLLAQMRRISPFS | 360 |
| K156A | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEAKYSPCAW | |
| | | | EVVRAEIMRSFSLSTNLQESLRSKE | |
| IFNA2b_L49P_ | 512 | 2.46 | CDLPQTHSLGSRRTLMLLAQMRRISPFS | 361 |
| S183L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQELLRSKE | |
| IFNA2b_M44N_ | 511 | 2.42 | CDLPQTHSLGSRRTLMLLAQNRRISLFS | 362 |
| K187D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSDE | |
| IFNA2b_L49P_ | 509 | 2.36 | CDLPQTHSLGSRRTLMLLAQMRRISPFS | 363 |
| R185M | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLMSKE | |
| IFNA2b_L49P_ | 505 | 2.42 | CDLPQTHSLGSRRTLMLLAQMRRISPFS | 364 |
| R185N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLNSKE | |
| IFNA2b_M44N_ | 494 | 2.02 | CDLPQTHSLGSRRTLMLLAQNRRISLFS | 365 |
| A168L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRLEIMRSFSLSTNLQESLRSKE | |
| IFNA2b_L49P_ | 492 | 2.23 | CDLPQTHSLGSRRTLMLLAQMRRISPFS | 366 |
| S175L | | | CLKDRHDFGFPQEEFGNQFQKAETIPVL | |
| | | | HEMIQQIFNLESTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFLLSTNLQESLRSKE | |
| IFNA2b_L49P_ | 479 | 2.60 | CDLPQTHSLGSRRTLMLLAQMRRISPFS | 367 |
| R185Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLQSKE | |
| IFNA2b_L49P_ | 475 | 2.27 | CDLPQTHSLGSRRTLMLLAQMRRISPFS | 368 |
| K187G | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSGE | |
| IFNA2b_M44N_ | 475 | 2.00 | CDLPQTHSLGSRRTLMLLAQNRRISLFS | 369 |
| S177E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLETNLQESLRSKE | |
| IFNA2b_L49P_ | 467 | 2.07 | CDLPQTHSLGSRRTLMLLAQMRRISPFS | 370 |
| W163L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAL | |
| | | | EVVRAEIMRSFSLSTNLQESLRSKE | |
| IFNA2b_L49P_ | 455 | 2.06 | CDLPQTHSLGSRRTLMLLAQMRRISPFS | 371 |
| R185W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLWSKE | |
| IFNA2b_L49P_ | 451 | 2.26 | CDLPQTHSLGSRRTLMLLAQMRRISPFS | 372 |
| K187T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSTE | |
| IFNA2b_L49P_ | 448 | 2.39 | CDLPQTHSLGSRRTLMLLAQMRRISPFS | 373 |
| T109W | | | CLKDRHDFGFPQEEFGNQFQKAETIPVL | |
| | | | HEMIQQIFNLFSTKDSSAAWDETLLDKF | |
| | | | YWELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_M44N_ | 444 | 2.06 | CDLPQTHSLGSRRTLMLLAQNRRISLFS | 374 |
| P160E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSECAW | |
| | | | EVVRAEIMRSFSLSTNLQESLRSKE | |
| IFNA2b_M44N_ | 438 | 2.03 | CDLPQTHSLGSRRTLMLLAQNRRISLFS | 375 |
| K106T | | | CLKDRHDFGFPQEEFGNQFQKAETIPVL | |
| | | | HEMIQQIFNLFSTKDSSAAWDETLLDTF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 431 | 2.33 | CDLPQTHSLGSRRTLMLLAQMRRISPFS | 376 |
| R185T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLTSKE | |
| IFNA2b_L49P_ | 429 | 2.14 | CDLPQTHSLGSRRTLMLLAQMRRISPFS | 377 |
| S175I | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFILSTNLQESLRSKE | |
| IFNA2b_L49P_ | 423 | 2.22 | CDLPQTHSLGSRRTLMLLAQMRRISPFS | 378 |
| S173F | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRFFSLSTNLQESLRSKE | |
| IFNA2b_M44N_ | 418 | 2.36 | CDLPQTHSLGSRRTLMLLAQNRRISLFS | 379 |
| R185L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLLSKE | |
| IFNA2b_L49P_ | 411 | 2.51 | CDLPQTHSLGSRRTLMLLAQMRRISPFS | 380 |
| S183E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQEELRSKE | |
| IFNA2b_L49P_ | 403 | 2.25 | CDLPQTHSLGSRRTLMLLAQMRRISPFS | 381 |
| K187N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSNE | |
| IFNA2b_M44N_ | 403 | 2.10 | CDLPQTHSLGSRRTLMLLAQNRRISLFS | 382 |
| K187F | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSFE | |
| IFNA2b_L49P_ | 400 | 2.24 | CDLPQTHSLGSRRTLMLLAQMRRISPFS | 383 |
| S159D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYDPCAW | |
| | | | EVVRAEIMRSFSLSTNLQESLRSKE | |
| IFNA2b_M44N_ | 392 | 2.05 | CDLPQTHSLGSRRTLMLLAQNRRISLFS | 384 |
| K106I | | | CLKDRHDFGFPQEEFGNQFQKAETIPVL | |
| | | | HEMIQQIFNLFSTKDSSAAWDETLLDIF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 391 | 2.01 | CDLPQTHSLGSRRTLMLLAQMRRISPFS | 385 |
| S175Y | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFYLSTNLQESLRSKE | |
| IFNA2b_L49P_ | 383 | 2.22 | CDLPQTHSLGSRRTLMLLAQMRRISPFS | 386 |
| L184N | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESNRSKE | |
| IFNA2b_L49P_ | 381 | 2.32 | CDLPQTHSLGSRRTLMLLAQMRRISPFS | 387 |
| I170L | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAELMRSFSLSTNLQESLRSKE | |
| IFNA2b_L49P_ | 381 | 2.10 | CDLPQTHSLGSRRTLMLLAQMRRISPFS | 388 |
| V166S | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVSRAEIMRSFSLSTNLQESLRSKE | |
| IFNA2b_L49P_ | 381 | 2.30 | CDLPQTHSLGSRRTLMLLAQMRRISPFS | 389 |
| K187A | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSAE | |
| IFNA2b_L49P_ | 373 | 2.10 | CDLPQTHSLGSRRTLMLLAQMRRISPFS | 390 |
| S186D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRDKE | |
| IFNA2b_L49P_ | 373 | 2.13 | CDLPQTHSLGSRRTLMLLAQMRRISPFS | 391 |
| L111F | | | CLKDRHDFGFPQEEFGNQFQKAETIPVL | |
| | | | HEMIQQIFNLFSTKDSSAAWDETLLDKF | |
| | | | YTEFYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 366 | 2.16 | CDLPQTHSLGSRRTLMLLAQMRRISPFS | 392 |
| K154Q | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLQEKKYSPCAW | |
| | | | EVVRAEIMRSFSLSTNLQESLRSKE | |
| IFNA2b_L49P_ | 363 | 2.06 | CDLPQTHSLGSRRTLMLLAQMRRISPFS | 393 |
| K106T | | | CLKDRHDFGFPQEEFGNQFQKAETIPVL | |
| | | | HEMIQQIFNLFSTKDSSAAWDETLLDTF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 363 | 2.02 | CDLPQTHSLGSRRTLMLLAQMRRISPFS | 394 |
| E136D | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | DDSILAVRKYFQRITLYLKEKKYSPCAW | |
| | | | EVVRAEIMRSFSLSTNLQESLRSKE | |
| IFNA2b_L49P_ | 359 | 2.10 | CDLPQTHSLGSRRTLMLLAQMRRISPFS | 395 |
| K106M | | | CLKDRHDFGFPQEEFGNQFQKAETIPVL | |
| | | | HEMIQQIFNLFSTKDSSAAWDETLLDMF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 356 | 2.05 | CDLPQTHSLGSRRTLMLLAQMRRISPFS | 396 |
| F107W | | | CLKDRHDFGFPQEEFGNQFQKAETIPVL | |
| | | | HEMIQQIFNLFSTKDSSAAWDETLLDKW | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_M44N_ | 356 | 2.30 | CDLPQTHSLGSRRTLMLLAQNRRISLFS | 397 |
| R185Y | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLYSKE | |
| IFNA2b_M44N_ | 354 | 2.17 | CDLPQTHSLGSRRTLMLLAQNRRISLFS | 398 |
| V165I | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EIVRAEIMRSFSLSTNLQESLRSKE | |
| IFNA2b_M44N_ | 354 | 2.13 | CDLPQTHSLGSRRTLMLLAQNRRISLFS | 399 |
| K187W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSWE | |
| IFNA2b_L49P_ | 350 | 2.38 | CDLPQTHSLGSRRTLMLLAQMRRISPFS | 400 |
| R185I | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLISKE | |
| IFNA2b_M44N_ | 350 | 2.44 | CDLPQTHSLGSRRTLMLLAQNRRISLFS | 401 |
| R185T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLTSKE | |
| IFNA2b_M44N_ | 347 | 2.32 | CDLPQTHSLGSRRTLMLLAQNRRISLFS | 402 |
| R185V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLVSKE | |
| IFNA2b_M44N_ | 341 | 2.20 | CDLPQTHSLGSRRTLMLLAQNRRISLFS | 403 |
| Q113A | | | CLKDRHDFGFPQEEFGNQFQKAETIPVL | |
| | | | HEMIQQIFNLFSTKDSSAAWDETLLDKF | |
| | | | YTELYAQLNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_M44N_ | 340 | 2.06 | CDLPQTHSLGSRRTLMLLAQNRRISLFS | 404 |
| T178K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSKNLQESLRSKE | |
| IFNA2b_L49P_ | 340 | 2.04 | CDLPQTHSLGSRRTLMLLAQMRRISPFS | 405 |
| K156V | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEVKYSPCAW | |
| | | | EVVRAEIMRSFSLSTNLQESLRSKE | |
| IFNA2b_L49P_ | 335 | 2.16 | CDLPQTHSLGSRRTLMLLAQMRRISPFS | 406 |
| K187S | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQESLRSSE | |
| IFNA2b_M44N_ | 334 | 2.06 | CDLPQTHSLGSRRTLMLLAQNRRISLFS | 407 |
| Q114I | | | CLKDRHDFGFPQEEFGNQFQKAETIPVL | |
| | | | HEMIQQIFNLFSTKDSSAAWDETLLDKF | |
| | | | YTELYQILNDLEACVIQGVGVTETPLMK | |
| | | | EDSILAVRKYFQRITLYLKEKKYSPCAW | |
| | | | EVVRAEIMRSFSLSTNLQESLRSKE | |
| IFNA2b_L49P_ | 331 | 2.09 | CDLPQTHSLGSRRTLMLLAQMRRISPFS | 408 |
| T131E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTEEPLMK | |
| | | | EDSILAVRKYFQRITLYLKEKKYSPCAW | |
| | | | EVVRAEIMRSFSLSTNLQESLRSKE | |
| IFNA2b_M44N_ | 329 | 2.14 | CDLPQTHSLGSRRTLMLLAQNRRISLFS | 409 |
| S183E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSTNLQEELRSKE | |
| IFNA2b_L49P_ | 328 | 2.26 | CDLPQTHSLGSRRTLMLLAQMRRISPFS | 410 |
| Y152W | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLWLKEKKYSPCAW | |
| | | | EVVRAEIMRSFSLSTNLQESLRSKE | |
| IFNA2b_L49P_ | 326 | 2.38 | CDLPQTHSLGSRRTLMLLAQMRRISPFS | 411 |
| V128E | | | CLKDRHDFGFPQEEFGNQFQKAETIPVL | |
| | | | HEMIQQIFNLFSTKDSSAAWDETLLDKF | |
| | | | YTELYQQLNDLEACVIQGVGETETPLMK | |
| | | | EDSILAVRKYFQRITLYLKEKKYSPCAW | |
| | | | EVVRAEIMRSFSLSTNLQESLRSKE | |
| IFNA2b_L49P_ | 325 | 2.14 | CDLPQTHSLGSRRTLMLLAQMRRISPFS | 412 |
| E155K | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKKKKYSPCAW | |
| | | | EVVRAEIMRSFSLSTNLQESLRSKE | |
| IFNA2b_L49P_ | 325 | 2.07 | CDLPQTHSLGSRRTLMLLAQMRRISPFS | 413 |
| S175T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFTLSTNLQESLRSKE | |
| IFNA2b_L49P_ | 323 | 2.21 | CDLPQTHSLGSRRTLMLLAQMRRISPFS | 414 |
| E155R | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKRKKYSPCAW | |
| | | | EVVRAEIMRSFSLSTNLQESLRSKE | |
| IFNA2b_L49P_ | 323 | 2.08 | CDLPQTHSLGSRRTLMLLAQMRRISPFS | 415 |
| V166T | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVTRAEIMRSFSLSTNLQESLRSKE | |
| IFNA2b_L49P_ | 319 | 2.12 | CDLPQTHSLGSRRTLMLLAQMRRISPFS | 416 |
| T178F | | | CLKDRHDFGFPQEEFGNQFQKAETIPVL | |
| | | | HEMIQQIFNLFSTKDSSAAWDETLLDKF | |
| | | | YTELYQQLNDLEACVIQGVGVTETPLMK | |
| | | | EDSILAVRKYFQRITLYLKEKKYSPCAW | |
| | | | EVVRAEIMRSFSLSFNLQESLRSKE | |

Table 4 below provides affinities between human IFNA2 detuned variants and mouse IFNAR2 as measured by AlphaSeq™. Any double mutant variant found to bind mouse IFNAR2 with half-log weaker affinity than the wild-type human IFNA2 polypeptide (407 nM for M44N double mutants and 63 nM for L49P double mutants) and found to have an average expression bin>2.0, but that did not show decreased affinity to human IFNAR2 was included in Table 4. Table 4 is sorted in order of descending Kd, with the highest values being the weakest binders. Table 4 also provides the polypeptide sequences of the 22 IFNA2 variants comprising two amino acid substitutions as SEQ ID NOs 417-438. The polypeptide sequences describe the mature IFNA2b protein, which does not include a 23-amino acid signal peptide that is cleaved from the N-terminus to produce the mature protein. The amino acid sequence of mouse IFNAR2 is provided below:

(SEQ ID NO: 3)
MRSRCTVSAVGLLSLCLVVSASLETITPSAFDGYPDEPCTINITIR
NSRLILSWELENKSGPPANYTLWYTVMSKDENLTKVKNCSDTTKSS
CDVTDKWLEGMESYVVAIVIVHRGDLTVCRCSDYIVPANAPLEPPE
FEIVGFTDHINVTMEFPPVTSKIIQEKMKTTPFVIKEQIGDSVRKK
HEPKVNNVTGNFTFVLRDLLPKTNYCVSLYFDDDPAIKSPLKCIVL
QPGQESGLSESAIVGITTSCLVVMVFVSTIVMLKRIGYICLKDNLP
NVLNFRHFLTWIIPERSPSEAIDRLEIIPTNKKKRLWNYDYEDGSD
SDEEVPTASVTGYTMHGLTGKPLQQTSDTSASPEDPLHEEDSGAEE
SDEAGAGAGAEPELPTEAGAGPSEDPTGPYERRKSVLEDSFPREDN
SSMDEPGDNIIFNVNLNSVFLRVLHDEDASETLSLEEDTILLDEGP
QRTESDLRIAGGDRTQPPLPSLPSQDLWTEDGSSEKSDTSDSDADV
GDGYIMR

TABLE 4
IFNA2 Double Mutants with Detuned Affinity to Mouse IFNAR2

| | Affinity | | | |
|---|---|---|---|---|
| | to mouse | Average | | SEQ |
| | IFNAR2 | expression | | ID |
| Variant | (nM) | bin | Polypeptide Sequence | NO |
| IFNA2b_M44N_ | 10825 | 2.21 | CDLPQTHSLGSRRTLMLLAQNRRISLFSCLKDRHD | 417 |
| A168W | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRWEIMRSFSLSTNLQESLRSKE | |
| IFNA2b_L49P_ | 3004 | 2.31 | CDLPQTHSLGSRRTLMLLAQMRRISPFSCLKDRHD | 418 |
| R167T | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVTAEIMRSFSLSTNLQESLRSKE | |
| IFNA2b_L49P_ | 1946 | 2.35 | CDLPQTHSLGSRRTLMLLAQMRRISPFSCLKDRHD | 419 |
| R167L | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVLAEIMRSFSLSTNLQESLRSKE | |
| IFNA2b_L49P_ | 1735 | 2.27 | CDLPQTHSLGSRRTLMLLAQMRRISPFSCLKDRHD | 420 |
| R167V | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVVAEIMRSFSLSTNLQESLRSKE | |
| IFNA2b_L49P_ | 1465 | 2.04 | CDLPQTHSLGSRRTLMLLAQMRRISPFSCLKDRHD | 421 |
| A168Y | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRYEIMRSFSLSTNLQESLRSKE | |
| IFNA2b_M44N_ | 1129 | 2.39 | CDLPQTHSLGSRRTLMLLAQNRRISLFSCLKDRHD | 422 |
| K135I | | | FGFPQEEFGNQFQKAETIPVLHEMIQQIFNLFSTK | |
| | | | DSSAAWDETLLDKFYTELYQQLNDLEACVIQGVGV | |
| | | | TETPLMIEDSILAVRKYFQRITLYLKEKKYSPCAW | |
| | | | EVVRAEIMRSFSLSTNLQESLRSKE | |
| IFNA2b_M44N_ | 825 | 2.03 | CDLPQTHSLGSRRTLMLLAQNRRISLFSCLKDRHD | 423 |
| E130M | | | FGFPQEEFGNQFQKAETIPVLHEMIQQIFNLFSTK | |
| | | | DSSAAWDETLLDKFYTELYQQLNDLEACVIQGVGV | |
| | | | TMTPLMKEDSILAVRKYFQRITLYLKEKKYSPCAW | |
| | | | EVVRAEIMRSFSLSTNLQESLRSKE | |
| IFNA2b_L49P_ | 769 | 2.17 | CDLPQTHSLGSRRTLMLLAQMRRISPFSCLKDRHD | 424 |
| G127D | | | FGFPQEEFGNQFQKAETIPVLHEMIQQIFNLFSTK | |
| | | | DSSAAWDETLLDKFYTELYQQLNDLEACVIQGVDV | |
| | | | TETPLMKEDSILAVRKYFQRITLYLKEKKYSPCAW | |
| | | | EVVRAEIMRSFSLSTNLQESLRSKE | |
| IFNA2b_L49P_ | 708 | 2.30 | CDLPQTHSLGSRRTLMLLAQMRRISPFSCLKDRHD | 425 |
| R167I | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVIAEIMRSFSLSTNLQESLRSKE | |
| IFNA2b_M44N_ | 591 | 2.06 | CDLPQTHSLGSRRTLMLLAQNRRISLFSCLKDRHD | 426 |
| A120T | | | FGFPQEEFGNQFQKAETIPVLHEMIQQIFNLFSTK | |
| | | | DSSAAWDETLLDKFYTELYQQLNDLETCVIQGVGV | |
| | | | TETPLMKEDSILAVRKYFQRITLYLKEKKYSPCAW | |
| | | | EVVRAEIMRSFSLSTNLQESLRSKE | |
| IFNA2b_L49P_ | 586 | 2.32 | CDLPQTHSLGSRRTLMLLAQMRRISPFSCLKDRHD | 427 |
| A168D | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRDEIMRSFSLSTNLQESLRSKE | |
| IFNA2b_M44N_ | 520 | 2.43 | CDLPQTHSLGSRRTLMLLAQNRRISLFSCLKDRHD | 428 |
| E188R | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRAEIMRSFSLSTNLQESLRSKR | |
| IFNA2b_M44N_ | 512 | 2.19 | CDLPQTHSLGSRRTLMLLAQNRRISLFSCLKDRHD | 429 |
| V126E | | | FGFPQEEFGNQFQKAETIPVLHEMIQQIFNLFSTK | |
| | | | DSSAAWDETLLDKFYTELYQQLNDLEACVIQGEGV | |
| | | | TETPLMKEDSILAVRKYFQRITLYLKEKKYSPCAW | |
| | | | EVVRAEIMRSFSLSTNLQESLRSKE | |
| IFNA2b_M44N_ | 486 | 2.02 | CDLPQTHSLGSRRTLMLLAQNRRISLFSCLKDRHD | 430 |
| K106L | | | FGFPQEEFGNQFQKAETIPVLHEMIQQIFNLFSTK | |
| | | | DSSAAWDETLLDLFYTELYQQLNDLEACVIQGVGV | |
| | | | TETPLMKEDSILAVRKYFQRITLYLKEKKYSPCAW | |
| | | | EVVRAEIMRSFSLSTNLQESLRSKE | |
| IFNA2b_L49P_ | 480 | 2.16 | CDLPQTHSLGSRRTLMLLAQMRRISPFSCLKDRHD | 431 |
| L140N | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSINAVRKYFQRITLYLKEKKYSPCAW | |
| | | | EVVRAEIMRSFSLSTNLQESLRSKE | |
| IFNA2b_M44N_ | 472 | 2.21 | CDLPQTHSLGSRRTLMLLAQNRRISLFSCLKDRHD | 432 |
| V128G | | | FGFPQEEFGNQFQKAETIPVLHEMIQQIFNLESTK | |
| | | | DSSAAWDETLLDKFYTELYQQLNDLEACVIQGVGG | |
| | | | TETPLMKEDSILAVRKYFQRITLYLKEKKYSPCAW | |
| | | | EVVRAEIMRSFSLSTNLQESLRSKE | |
| IFNA2b_M44N_ | 413 | 2.03 | CDLPQTHSLGSRRTLMLLAQNRRISLFSCLKDRHD | 433 |
| L180E | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRAEIMRSFSLSTNEQESLRSKE | |
| IFNA2b_L49P_ | 267 | 2.32 | CDLPQTHSLGSRRTLMLLAQMRRISPFSCLKDRHD | 434 |
| A168F | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRFEIMRSFSLSTNLQESLRSKE | |
| IFNA2b_L49P_ | 237 | 2.15 | CDLPQTHSLGSRRTLMLLAQMRRISPFSCLKDRHD | 435 |
| A168W | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRWEIMRSFSLSTNLQESLRSKE | |
| IFNA2b_L49P_ | 79 | 2.24 | CDLPQTHSLGSRRTLMLLAQMRRISPFSCLKDRHD | 436 |
| S175D | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRAEIMRSFDLSTNLQESLRSKE | |
| IFNA2b_L49P_ | 73 | 2.33 | CDLPQTHSLGSRRTLMLLAQMRRISPFSCLKDRHD | 437 |
| K156Q | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEQKYSPCAW | |
| | | | EVVRAEIMRSFSLSTNLQESLRSKE | |
| IFNA2b_L49P_ | 67 | 2.39 | CDLPQTHSLGSRRTLMLLAQMRRISPFSCLKDRHD | 438 |
| A168H | | | FGFPQEEFGNQFQKAETIPVLHEMIQQIFNLFSTK | |
| | | | DSSAAWDETLLDKFYTELYQQLNDLEACVIQGVGV | |
| | | | TETPLMKEDSILAVRKYFQRITLYLKEKKYSPCAW | |
| | | | EVVRHEIMRSFSLSTNLQESLRSKE | |

Table 5 below provides affinities between human IFNA2 detuned variants and both mouse IFNAR2 and human IFNAR2 as measured by AlphaSeq™. For inclusion in Table 5, a double mutant variant must have bound human IFNAR2 with an affinity weaker than 100 nM and mouse IFNAR2 with an affinity weaker than 407 nM (for M44N double mutants) or weaker than 63 nM (for L49P double mutants), and found to have an average expression bin>2.0. Table 5 is sorted in order of descending Kd for mouse IFNAR2, with the highest values representing the lowest affinity of IFNA2 for mouse IFNAR2. Table 5 also provides the polypeptide sequences of the 100 IFNA2 variants comprising two amino acid substitutions as SEQ ID NOs 439-538. The polypeptide sequences describe the mature IFNA2b protein, which does not include a 23-amino acid signal peptide that is cleaved from the N-terminus to produce the mature protein.

TABLE 5
IFNA2 Double Mutants with Detuned
Affinity to Mouse IFNAR2 and Human IFNAR2

| | Affinity | Affinity | | | |
|---|---|---|---|---|---|
| | to | to | | | |
| | human | mouse | Average | | SEQ |
| | IFNAR2 | IFNAR2 | expression | | ID |
| Variant | (nM) | (nM) | bin | Polypeptide Sequence | NO |
| IFNA2b_M44N_ | 3128 | 107006 | 2.15 | CDLPQTHSLGSRRTLMLLAQNRRISLF | 439 |
| R172N | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMNSFSLSTNLQESLR | |
| | | | | SKE | |
| IFNA2b_L49P_ | 50882 | 65847 | 2.14 | CDLPQTHSLGSRRTLMLLAQMRRISPF | 440 |
| R167S | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVSAEIMRSFSLSTNLQESLR | |
| | | | | SKE | |
| IFNA2b_L49P_ | 124080 | 52552 | 2.27 | CDLPQTHSLGSRRTLMLLAQMRRISPF | 441 |
| R167G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVGAEIMRSFSLSTNLQESLR | |
| | | | | SKE | |
| IFNA2b_L49P_ | 134097 | 47292 | 2.18 | CDLPQTHSLGSRRTLMLLAQMRRISPF | 442 |
| R172E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMESFSLSTNLQESLR | |
| | | | | SKE | |
| IFNA2b_M44N_ | 20953 | 37090 | 2.09 | CDLPQTHSLGSRRTLMLLAQNRRISLF | 443 |
| R172G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMGSFSLSTNLQESLR | |
| | | | | SKE | |
| IFNA2b_M44N_ | 101711 | 34462 | 2.13 | CDLPQTHSLGSRRTLMLLAQNRRISLF | 444 |
| S159D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | DPCAWEVVRAEIMRSFSLSTNLQESLR | |
| | | | | SKE | |
| IFNA2b_L49P_ | 41312 | 34403 | 2.25 | CDLPQTHSLGSRRTLMLLAQMRRISPF | 445 |
| V165G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEGVRAEIMRSFSLSTNLQESLR | |
| | | | | SKE | |
| IFNA2b_L49P_ | 22212 | 30329 | 2.26 | CDLPQTHSLGSRRTLMLLAQMRRISPF | 446 |
| A168E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVREEIMRSFSLSTNLQESLR | |
| | | | | SKE | |
| IFNA2b_L49P_ | 42706 | 30122 | 2.16 | CDLPQTHSLGSRRTLMLLAQMRRISPF | 447 |
| V165K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEKVRAEIMRSFSLSTNLQESLR | |
| | | | | SKE | |
| IFNA2b_M44N_ | 67968 | 26832 | 2.69 | CDLPQTHSLGSRRTLMLLAQNRRISLF | 448 |
| K187R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SRE | |
| IFNA2b_M44N_ | 93380 | 26262 | 2.12 | CDLPQTHSLGSRRTLMLLAQNRRISLF | 449 |
| R143I | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVIKYFQRITLYLKEKKY | |
| | | | | SPCAWEVVRAEIMRSFSLSTNLQESLR | |
| | | | | SKE | |
| IFNA2b_L49P_ | 52289 | 26107 | 2.26 | CDLPQTHSLGSRRTLMLLAQMRRISPF | 450 |
| V165D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEDVRAEIMRSFSLSTNLQESLR | |
| | | | | SKE | |
| IFNA2b_M44N_ | 42746 | 24351 | 2.07 | CDLPQTHSLGSRRTLMLLAQNRRISLF | 451 |
| K156E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEEKY | |
| | | | | SPCAWEVVRAEIMRSFSLSTNLQESLR | |
| | | | | SKE | |
| IFNA2b_M44N_ | 27624 | 23252 | 2.09 | CDLPQTHSLGSRRTLMLLAQNRRISLF | 452 |
| A168K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRKEIMRSFSLSTNLQESLR | |
| | | | | SKE | |
| IFNA2b_L49P_ | 38495 | 22900 | 2.19 | CDLPQTHSLGSRRTLMLLAQMRRISPF | 453 |
| V165F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEFVRAEIMRSFSLSTNLQESLR | |
| | | | | SKE | |
| IFNA2b_L49P_ | 35042 | 21137 | 2.13 | CDLPQTHSLGSRRTLMLLAQMRRISPF | 454 |
| R172D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMDSFSLSTNLQESLR | |
| | | | | SKE | |
| IFNA2b_M44N_ | 9219 | 19977 | 2.11 | CDLPQTHSLGSRRTLMLLAQNRRISLF | 455 |
| A168Q | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRQEIMRSFSLSTNLQESLR | |
| | | | | SKE | |
| IFNA2b_L49P_ | 34139 | 18640 | 2.40 | CDLPQTHSLGSRRTLMLLAQMRRISPF | 456 |
| V165Q | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEQVRAEIMRSFSLSTNLQESLR | |
| | | | | SKE | |
| IFNA2b_L49P_ | 123608 | 18095 | 2.28 | CDLPQTHSLGSRRTLMLLAQMRRISPF | 457 |
| R172N | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMNSFSLSTNLQESLR | |
| | | | | SKE | |
| IFNA2b_M44N_ | 21921 | 17998 | 2.07 | CDLPQTHSLGSRRTLMLLAQNRRISLF | 458 |
| A168D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRDEIMRSFSLSTNLQESLR | |
| | | | | SKE | |
| IFNA2b_M44N_ | 11250 | 17982 | 2.16 | CDLPQTHSLGSRRTLMLLAQNRRISLF | 459 |
| K156Q | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEQKY | |
| | | | | SPCAWEVVRAEIMRSFSLSTNLQESLR | |
| | | | | SKE | |
| IFNA2b_L49P_ | 27477 | 16839 | 2.29 | CDLPQTHSLGSRRTLMLLAQMRRISPF | 460 |
| V165S | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWESVRAEIMRSFSLSTNLQESLR | |
| | | | | SKE | |
| IFNA2b_M44N_ | 505 | 16815 | 2.48 | CDLPQTHSLGSRRTLMLLAQNRRISLF | 461 |
| S186K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | KKE | |
| IFNA2b_L49P_ | 25777 | 16712 | 2.37 | CDLPQTHSLGSRRTLMLLAQMRRISPF | 462 |
| V165Y | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEYVRAEIMRSFSLSTNLQESLR | |
| | | | | SKE | |
| IFNA2b_L49P_ | 33124 | 16435 | 2.31 | CDLPQTHSLGSRRTLMLLAQMRRISPF | 463 |
| V165E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEEVRAEIMRSFSLSTNLQESLR | |
| | | | | SKE | |
| IFNA2b_M44N_ | 2347 | 15815 | 2.26 | CDLPQTHSLGSRRTLMLLAQNRRISLF | 464 |
| E155K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKKKKY | |
| | | | | SPCAWEVVRAEIMRSFSLSTNLQESLR | |
| | | | | SKE | |
| IFNA2b_M44N_ | 32781 | 12301 | 2.01 | CDLPQTHSLGSRRTLMLLAQNRRISLF | 465 |
| S177D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLDTNLQESLR | |
| | | | | SKE | |
| IFNA2b_M44N_ | 37739 | 11682 | 2.16 | CDLPQTHSLGSRRTLMLLAQNRRISLF | 466 |
| M171E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIERSFSLSTNLQESLR | |
| | | | | SKE | |
| IFNA2b_M44N_ | 4421 | 10938 | 2.05 | CDLPQTHSLGSRRTLMLLAQNRRISLF | 467 |
| R172D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMDSFSLSTNLQESLR | |
| | | | | SKE | |
| IFNA2b_L49P_ | 26763 | 10461 | 2.20 | CDLPQTHSLGSRRTLMLLAQMRRISPF | 468 |
| V165M | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEMVRAEIMRSFSLSTNLQESLR | |
| | | | | SKE | |
| IFNA2b_L49P_ | 31708 | 9946 | 2.22 | CDLPQTHSLGSRRTLMLLAQMRRISPF | 469 |
| R172W | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMWSFSLSTNLQESLR | |
| | | | | SKE | |
| IFNA2b_L49P_ | 29326 | 8964 | 2.21 | CDLPQTHSLGSRRTLMLLAQMRRISPF | 170 |
| V165R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWERVRAEIMRSFSLSTNLQESLR | |
| | | | | SKE | |
| IFNA2b_M44N_ | 1846 | 8771 | 2.02 | CDLPQTHSLGSRRTLMLLAQNRRISLF | 471 |
| R172W | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMWSFSLSTNLQESLR | |
| | | | | SKE | |
| IFNA2b_L49P_ | 35529 | 8552 | 2.04 | CDLPQTHSLGSRRTLMLLAQMRRISPF | 472 |
| R172Q | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMQSFSLSTNLQESLR | |
| | | | | SKE | |
| IFNA2b_M44N_ | 3370 | 7665 | 2.12 | CDLPQTHSLGSRRTLMLLAQNRRISLF | 473 |
| E155R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKRKKY | |
| | | | | SPCAWEVVRAEIMRSFSLSTNLQESLR | |
| | | | | SKE | |
| IFNA2b_M44N_ | 1656 | 7013 | 2.02 | CDLPQTHSLGSRRTLMLLAQNRRISLF | 474 |
| R167T | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVTAEIMRSFSLSTNLQESLR | |
| | | | | SKE | |
| IFNA2b_L49P_ | 32411 | 6867 | 2.17 | CDLPQTHSLGSRRTLMLLAQMRRISPF | 475 |
| R172A | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMASFSLSTNLQESLR | |
| | | | | SKE | |
| IFNA2b_L49P_ | 27441 | 6436 | 2.17 | CDLPQTHSLGSRRTLMLLAQMRRISPF | 476 |
| R167W | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVWAEIMRSFSLSTNLQESLR | |
| | | | | SKE | |
| IFNA2b_L49P_ | 47048 | 6206 | 2.07 | CDLPQTHSLGSRRTLMLLAQMRRISPF | 477 |
| R172T | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMTSFSLSTNLQESLR | |
| | | | | SKE | |
| IFNA2b_L49P_ | 35911 | 6189 | 2.31 | CDLPQTHSLGSRRTLMLLAQMRRISPF | 478 |
| R172S | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMSSFSLSTNLQESLR | |
| | | | | SKE | |
| IFNA2b_L49P_ | 17034 | 5952 | 2.23 | CDLPQTHSLGSRRTLMLLAQMRRISPF | 479 |
| V165T | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWETVRAEIMRSFSLSTNLQESLR | |
| | | | | SKE | |
| IFNA2b_M44N_ | 604 | 5816 | 2.17 | CDLPQTHSLGSRRTLMLLAQNRRISLF | 480 |
| G125Y | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQYVGVTET | |
| | | | | PLMKEDSILAVRKYFQRITLYLKEKKY | |
| | | | | SPCAWEVVRAEIMRSFSLSTNLQESLR | |
| | | | | SKE | |
| IFNA2b_M44N_ | 563 | 5776 | 2.03 | CDLPQTHSLGSRRTLMLLAQNRRISLF | 48. |
| R167A | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVAAEIMRSFSLSTNLQESLR | |
| | | | | SKE | |
| IFNA2b_M44N_ | 648 | 4896 | 2.08 | CDLPQTHSLGSRRTLMLLAQNRRISLF | 482 |
| R167I | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVIAEIMRSFSLSTNLQESLR | |
| | | | | SKE | |
| IFNA2b_L49P_ | 4124 | 4462 | 2.14 | CDLPQTHSLGSRRTLMLLAQMRRISPF | 483 |
| R167F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVFAEIMRSFSLSTNLQESLR | |
| | | | | SKE | |
| IFNA2b_M44N_ | 8262 | 4412 | 2.55 | CDLPQTHSLGSRRTLMLLAQNRRISLF | 484 |
| L180G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNGQESLR | |
| | | | | SKE | |
| IFNA2b_L49P_ | 42637 | 2765 | 2.05 | CDLPQTHSLGSRRTLMLLAQMRRISPF | 485 |
| V165N | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWENVRAEIMRSFSLSTNLQESLR | |
| | | | | SKE | |
| IFNA2b_L49P_ | 1892 | 2382 | 2.36 | CDLPQTHSLGSRRTLMLLAQMRRISPF | 486 |
| R167M | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVMAEIMRSFSLSTNLQESLR | |
| | | | | SKE | |
| IFNA2b_M44N_ | 670 | 2265 | 2.02 | CDLPQTHSLGSRRTLMLLAQNRRISLF | 487 |
| R167L | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVLAEIMRSFSLSTNLQESLR | |
| | | | | SKE | |
| IFNA2b_L49P_ | 76389 | 2257 | 2.48 | CDLPQTHSLGSRRTLMLLAQMRRISPF | 488 |
| R172G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMGSFSLSTNLQESLR | |
| | | | | SKE | |
| IFNA2b_L49P_ | 1945 | 2196 | 2.16 | CDLPQTHSLGSRRTLMLLAQMRRISPF | 489 |
| R167A | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVAAEIMRSFSLSTNLQESLR | |
| | | | | SKE | |
| IFNA2b_M44N_ | 145711 | 2030 | 2.16 | CDLPQTHSLGSRRTLMLLAQNRRISLF | 490 |
| S173R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRRFSLSTNLQESLR | |
| | | | | SKE | |
| IFNA2b_M44N_ | 5759 | 1633 | 2.12 | CDLPQTHSLGSRRTLMLLAQNRRISLF | 491 |
| Q114K | | | | SCLKDRHDFGFPQEEFGNQFQKAETIP | |
| | | | | VLHEMIQQIFNLFSTKDSSAAWDETLL | |
| | | | | DKFYTELYQKLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SKE | |
| IFNA2b_M44N_ | 5644 | 1496 | 2.07 | CDLPQTHSLGSRRTLMLLAQNRRISLF | 492 |
| A168N | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRNEIMRSFSLSTNLQESLR | |
| | | | | SKE | |
| IFNA2b_L49P_ | 6476 | 1179 | 2.30 | CDLPQTHSLGSRRTLMLLAQMRRISPF | 493 |
| V165H | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEHVRAEIMRSFSLSTNLQESLR | |
| | | | | SKE | |
| IFNA2b_M44N_ | 2635 | 942 | 2.06 | CDLPQTHSLGSRRTLMLLAQNRRISLF | 494 |
| S138W | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDWILAVRKYFQRITLYLKEKKY | |
| | | | | SPCAWEVVRAEIMRSFSLSTNLQESLR | |
| | | | | SKE | |
| IFNA2b_M44N_ | 596 | 811 | 2.07 | CDLPQTHSLGSRRTLMLLAQNRRISLF | 495 |
| L184E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESER | |
| | | | | SKE | |
| IFNA2b_M44N_ | 997 | 802 | 2.35 | CDLPQTHSLGSRRTLMLLAQNRRISLF | 496 |
| A168H | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRHEIMRSFSLSTNLQESLR | |
| | | | | SKE | |
| IFNA2b_L49P_ | 6216 | 697 | 2.26 | CDLPQTHSLGSRRTLMLLAQMRRISPF | 497 |
| K135I | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMIEDSILAVRKYFQRITLYLKEKKY | |
| | | | | SPCAWEVVRAEIMRSFSLSTNLQESLR | |
| | | | | SKE | |
| IFNA2b_M44N_ | 584 | 584 | 2.09 | CDLPQTHSLGSRRTLMLLAQNRRISLF | 498 |
| T150L | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRILLYLKEKKY | |
| | | | | SPCAWEVVRAEIMRSFSLSTNLQESLR | |
| | | | | SKE | |
| IFNA2b_L49P_ | 12189 | 566 | 2.17 | CDLPQTHSLGSRRTLMLLAQMRRISPF | 499 |
| R172V | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMVSFSLSTNLQESLR | |
| | | | | SKE | |
| IFNA2b_M44N_ | 346 | 554 | 2.22 | CDLPQTHSLGSRRTLMLLAQNRRISLF | 500 |
| T178F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSENLQESLR | |
| | | | | SKE | |
| IFNA2b_M44N_ | 357 | 545 | 2.11 | CDLPQTHSLGSRRTLMLLAQNRRISLF | 501 |
| A168S | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRSEIMRSFSLSTNLQESLR | |
| | | | | SKE | |
| IFNA2b_M44N_ | 52438 | 541 | 2.08 | CDLPQTHSLGSRRTLMLLAQNRRISLF | 502 |
| L176R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSRSTNLQESLR | |
| | | | | SKE | |
| IFNA2b_M44N_ | 559 | 528 | 2.05 | CDLPQTHSLGSRRTLMLLAQNRRISLF | 503 |
| T150G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRIGLYLKEKKY | |
| | | | | SPCAWEVVRAEIMRSFSLSTNLQESLR | |
| | | | | SKE | |
| IFNA2b_L49P_ | 17279 | 518 | 2.37 | CDLPQTHSLGSRRTLMLLAQMRRISPF | 504 |
| S183W | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQEWLR | |
| | | | | SKE | |
| IFNA2b_M44N_ | 686 | 487 | 2.05 | CDLPQTHSLGSRRTLMLLAQNRRISLF | 505 |
| L176E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSESTNLQESLR | |
| | | | | SKE | |
| IFNA2b_M44N_ | 366 | 484 | 2.09 | CDLPQTHSLGSRRTLMLLAQNRRISLF | 506 |
| S175E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFELSTNLQESLR | |
| | | | | SKE | |
| IFNA2b_L49P_ | 2356 | 480 | 2.39 | CDLPQTHSLGSRRTLMLLAQMRRISPF | 507 |
| R167H | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVHAEIMRSFSLSTNLQESLR | |
| | | | | SKE | |
| IFNA2b_L49P_ | 38040 | 461 | 2.14 | CDLPQTHSLGSRRTLMLLAQMRRISPF | 508 |
| A168K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRKEIMRSFSLSTNLQESLR | |
| | | | | SKE | |
| IFNA2b_M44N_ | 602 | 420 | 2.43 | CDLPQTHSLGSRRTLMLLAQNRRISLF | 509 |
| S175D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFDLSTNLQESLR | |
| | | | | SKE | |
| IFNA2b_M44N_ | 330 | 416 | 2.18 | CDLPQTHSLGSRRTLMLLAQNRRISLF | 510 |
| K135G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMGEDSILAVRKYFQRITLYLKEKKY | |
| | | | | SPCAWEVVRAEIMRSFSLSTNLQESLR | |
| | | | | SKE | |
| IFNA2b_L49P_ | 8292 | 415 | 2.80 | CDLPQTHSLGSRRTLMLLAQMRRISPF | 511 |
| K187R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SRE | |
| IFNA2b_M44N_ | 1129 | 415 | 2.14 | CDLPQTHSLGSRRTLMLLAQNRRISLF | 512 |
| K187E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SEE | |
| IFNA2b_L49P_ | 361 | 321 | 2.31 | CDLPQTHSLGSRRTLMLLAQMRRISPF | 513 |
| A168Q | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRQEIMRSFSLSTNLQESLR | |
| | | | | SKE | |
| IFNA2b_L49P_ | 30426 | 310 | 2.40 | CDLPQTHSLGSRRTLMLLAQMRRISPF | 514 |
| R172I | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMISFSLSTNLQESLR | |
| | | | | SKE | |
| IFNA2b_L49P_ | 325 | 293 | 2.36 | CDLPQTHSLGSRRTLMLLAQMRRISPF | 515 |
| A168M | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRMEIMRSFSLSTNLQESLR | |
| | | | | SKE | |
| IFNA2b_L49P_ | 24117 | 273 | 2.14 | CDLPQTHSLGSRRTLMLLAQMRRISPF | 516 |
| R172F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMFSFSLSTNLQESLR | |
| | | | | SKE | |
| IFNA2b_L49P_ | 42476 | 227 | 2.05 | CDLPQTHSLGSRRTLMLLAQMRRISPF | 517 |
| R172M | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMMSFSLSTNLQESLR | |
| | | | | SKE | |
| IFNA2b_L49P_ | 16777 | 206 | 2.28 | CDLPQTHSLGSRRTLMLLAQMRRISPF | 518 |
| L176D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSDSTNLQESLR | |
| | | | | SKE | |
| IFNA2b_L49P_ | 37221 | 203 | 2.16 | CDLPQTHSLGSRRTLMLLAQMRRISPF | 519 |
| R172Y | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMYSFSLSTNLQESLR | |
| | | | | SKE | |
| IFNA2b_L49P_ | 32692 | 173 | 2.49 | CDLPQTHSLGSRRTLMLLAQMRRISPF | 520 |
| M171E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIERSFSLSTNLQESLR | |
| | | | | SKE | |
| IFNA2b_L49P_ | 1134 | 167 | 2.04 | CDLPQTHSLGSRRTLMLLAQMRRISPF | 521 |
| K156D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEDKY | |
| | | | | SPCAWEVVRAEIMRSFSLSTNLQESLR | |
| | | | | SKE | |
| IFNA2b_L49P_ | 3491 | 163 | 2.27 | CDLPQTHSLGSRRTLMLLAQMRRISPF | 522 |
| A168G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRGEIMRSFSLSTNLQESLR | |
| | | | | SKE | |
| IFNA2b_L49P_ | 41957 | 146 | 2.07 | CDLPQTHSLGSRRTLMLLAQMRRISPF | 523 |
| A168R | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRREIMRSFSLSTNLQESLR | |
| | | | | SKE | |
| IFNA2b_L49P_ | 39292 | 142 | 2.19 | CDLPQTHSLGSRRTLMLLAQMRRISPF | 524 |
| R172L | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMLSFSLSTNLQESLR | |
| | | | | SKE | |
| IFNA2b_L49P_ | 6913 | 114 | 2.56 | CDLPQTHSLGSRRTLMLLAQMRRISPF | 525 |
| S183F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQEFLR | |
| | | | | SKE | |
| IFNA2b_L49P_ | 5317 | 106 | 2.46 | CDLPQTHSLGSRRTLMLLAQMRRISPF | 526 |
| K187I | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SIE | |
| IFNA2b_L49P_ | 17237 | 106 | 2.01 | CDLPQTHSLGSRRTLMLLAQMRRISPF | 527 |
| M171D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIDRSFSLSTNLQESLR | |
| | | | | SKE | |
| IFNA2b_L49P_ | 2369 | 90 | 2.08 | CDLPQTHSLGSRRTLMLLAQMRRISPF | 528 |
| K187Y | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SYE | |
| IFNA2b_L49P_ | 2780 | 85 | 2.47 | CDLPQTHSLGSRRTLMLLAQMRRISPF | 529 |
| K187L | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SLE | |
| IFNA2b_L49P_ | 12068 | 84 | 2.25 | CDLPQTHSLGSRRTLMLLAQMRRISPF | 530 |
| N179G | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTGLQESLR | |
| | | | | SKE | |
| IFNA2b_L49P_ | 532 | 83 | 2.44 | CDLPQTHSLGSRRTLMLLAQMRRISPF | 531 |
| K156E | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEEKY | |
| | | | | SPCAWEVVRAEIMRSFSLSTNLQESLR | |
| | | | | SKE | |
| IFNA2b_L49P_ | 553 | 77 | 2.11 | CDLPQTHSLGSRRTLMLLAQMRRISPF | 532 |
| R167K | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVKAEIMRSFSLSTNLQESLR | |
| | | | | SKE | |
| IFNA2b_L49P_ | 2673 | 75 | 2.19 | CDLPQTHSLGSRRTLMLLAQMRRISPF | 533 |
| S183Y | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQEYLR | |
| | | | | SKE | |
| IFNA2b_L49P_ | 14447 | 75 | 2.31 | CDLPQTHSLGSRRTLMLLAQMRRISPF | 534 |
| E164D | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWDVVRAEIMRSFSLSTNLQESLR | |
| | | | | SKE | |
| IFNA2b_L49P_ | 14207 | 69 | 2.05 | CDLPQTHSLGSRRTLMLLAQMRRISPF | 535 |
| L176P | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSPSTNLQESLR | |
| | | | | SKE | |
| IFNA2b_L49P_ | 1115 | 67 | 2.27 | CDLPQTHSLGSRRTLMLLAQMRRISPF | 536 |
| K187F | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | SPCAWEVVRAEIMRSFSLSTNLQESLR | |
| | | | | SFE | |
| IFNA2b_L49P_ | 437 | 65 | 2.35 | CDLPQTHSLGSRRTLMLLAQMRRISPF | 537 |
| K156T | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKETKY | |
| | | | | SPCAWEVVRAEIMRSFSLSTNLQESLR | |
| | | | | SKE | |
| IFNA2b_L49P_ | 445 | 65 | 2.28 | CDLPQTHSLGSRRTLMLLAQMRRISPF | 538 |
| S159H | | | | SCLKDRHDFGFPQEEFGNQFQKAETIP | |
| | | | | VLHEMIQQIFNLFSTKDSSAAWDETLL | |
| | | | | DKFYTELYQQLNDLEACVIQGVGVTET | |
| | | | | PLMKEDSILAVRKYFQRITLYLKEKKY | |
| | | | | HPCAWEVVRAEIMRSFSLSTNLQESLR | |
| | | | | SKE | |

In some embodiments, the IFNA2 variant includes amino acid substitutions having one or more specific substitutions at one or more of positions H30, S31, L32, S34, R35, R36, L38, L40, L41, A42, Q43, M44, R45, R46, I47, S48, L49, F50, S51, L53, K54, D55, R56, H57, D58, F59, F61, P62, Q63, Q69, K72, V78, M82, Q84, I86, K93, A98, L103, K106, Y108, T109, E110, Q113, N116, N116, E119, A120, G125, V126, T129, P132, M134, I139, A141, R143, Y145, Q147, R148, E155, K156, K157, P160, V165, R167, A168, E169, I170, M171, R172, S173, S175, L176, S177, N179, S183, R185, S186, K187, and E188 in the wild-type IFNA2 amino acid sequence (SEQ ID NO: 1).

In some embodiments, the IFNA2 variant includes amino acid substitutions having one or more specific substitutions selected from the group consisting of: H30A, H30D, S31D, L32D, L32E, S34A, R35N, R36G, L38H, L38Y, L38G, L38P, L38S, L38T, L38Q, L38N, L40R, L40G, L40Q, L40D, L41H, L41D, L41K, L41G, L41A, A42G, A42M, Q43P, M44Q, R45P, R46P, R46D, R46F, R46Y, R46N, R46S, R46I, R46G, R46A, R46H, R46T, 147P, 147D, 147S, 147E, S48Y, S48H, L49H, L49I, L49K, L49V, L49Y, L49F, L49G, L49E, L49P, F50D, F50N, F50G, F50Q, F50S, F50M, F50H, F50A, S51D, S51E, L53D, L53E, L53G, L53A, L53N, L53V, L53S, K54L, K54I, K54M, D55T, D55N, D55Q, R56D, R56G, R56K, R56N, R56V, R56T, R56A, R56L, R56H, H57P, D58Q, F59Q, F59E, F591, F59N, F59A, F59G, F59T, F59Y, F61G, F61I, F61V, F61P, F61Q, F61A, F61S, P62I, Q63E, Q69S, K72D, V78E, V78G, M82W, M82K, M82R, M82G, M82S, 186L, K93E, K93V, A98E, L103E, L103D, K106L, K106D, Y108K, T109W, E110P, E110S, Q113W, N116W, N116M, N116F, N116Q, E119Y, E119F, A120M, G125V, V126W, V126Y, T129G, P132W, M134Y, I139Y, I139F, A141E, A141I, R143F, Q147S, R148Y, E155H, K156D, K156L, K156W, K157Y, P160E, P160F, P160W, P160Y, P160T, V165Y, V165E, V165H, V165K, V165W, V165F, V165Q, V165L, V165M, V165S, V165R, V165N, V165D, V165I, R167W, R167I, R167M, R167S, R167E, R167L, R167V, R167A, R167G, R167H, A168R, A168H, A168K, A168Y, A168G, A168F, A168D, A168M, A168Q, E169T, E169S, E169G, 1170L, M171R, M171S, M171T, M171N, M171A, M171Y, M171W, M171F, M171K, M171E, M171G, M171L, M171I, M171V, R172D, R172W, R172N, R172A, R172V, R172G, R172T, R172S, R172Y, R172L, R172M, R172K, S173K, S173R, S173H, S173E, S173N, S173W, S173Y, S175R, S175K, S175M, S175L, S175P, S175I, S175V, S175W, S175Y, S175G, S175E, S175Q, S175T, L176N, L176G, L176H, L176A, L176P, L176D, L176R, L176Q, L176E, L176V, S177D, S177R, N179G, S183W, S183F, S183M, S183E, S183Y, S183K, S183L, R185D, R185E, R185Q, S186L, S186I, S186D, K187R, K187L, K187E, K187Y, K187V, K187M, K187F, K187I, K187W, K187G, and E1881 in the wild-type IFNA2 amino acid sequence (SEQ ID NO: 1).

In some embodiments, the IFNA2 variant includes a combination of two specific amino acid substitutions, i.e., a double amino acid substitution, selected from the group consisting of: M44N+A168R, L49P+S175P, L49P+S173H, L49P+M171W, L49P+M171G, L49P+S173R, M44N+S175P, L49P+M171V, L49P+M171K, L49P+M171S, L49P+S175R, L49P+M171R, L49P+L176G, L49P+M171Y, L49P+L176N, L49P+S173Q, L49P+L176H, L49P+L176R, L49P+M171A, L49P+M171T, L49P+L176A, L49P+M171F, M44N+S175K, L49P+S175K, L49P+L176E, L49P+E169T, L49P+M171L, L49P+L176Q, L49P+K187E, L49P+S173N, M44N+L176H, M44N+S175R, L49P+S177N, L49P+S173W, L49P+S177D, L49P+R185D, L49P+E169S, L49P+S175G, L49P+S177L, L49P+R172K, L49P+R185E, L49P+K187M, L49P+K187W, L49P+S159Q, M44N+L176Q, M44N+R185E, L49P+S175Q, L49P+S175M, L49P+E155V, L49P+K187D, L49P+S175W, L49P+L176V, L49P+S173L, M44N+S175G, L49P+R185G, L49P+R143W, L49P+I149T, L49P+R185L, L49P+R185S, L49P+S177H, L49P+K106D, M44N+R185N, L49P+K187V, L49P+K106W, L49P+K135T, M44N+V128I, M44N+R185D, L49P+K106P, L49P+S183M, L49P+S183D, L49P+K156A, L49P+S183L, M44N+K187D, L49P+R185M, L49P+R185N, M44N+A168L, L49P+S175L, L49P+R185Q, L49P+K187G, M44N+S177E, L49P+W163L, L49P+R185W, L49P+K187T, L49P+T109W, M44N+P160E, M44N+K106T, L49P+R185T, L49P+S175I, L49P+S173F, M44N+R185L, L49P+S183E, L49P+K187N, M44N+K187F, L49P+S159D, M44N+K106I, L49P+S175Y, L49P+L184N, L49P+I170L, L49P+V166S, L49P+K187A, L49P+S186D, L49P+L111F, L49P+K154Q, L49P+K106T, L49P+E136D, L49P+K106M, L49P+F107W, M44N+R185Y, M44N+V165I, M44N+K187W, L49P+R185I, M44N+R185T, M44N+R185V, M44N+Q113A, M44N+T178K, L49P+K156V, L49P+K187S, M44N+Q114I, L49P+T131E, M44N+S183E, L49P+Y152W, L49P+V128E, L49P+E155K, L49P+S175T, L49P+E155R, L49P+V166T, and L49P+T178F in the wild-type IFNA2 amino acid sequence (SEQ ID NO: 1).

In some embodiments, the IFNA2 variant has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

### IFNA2 Fusion Proteins and Methods of Making

This disclosure provides isolated IFNA2 fusion proteins (e.g., antibody-IFNA2 fusion proteins) that have decreased or no detectable binding to the interferon alpha/beta receptor 2 (IFNAR2). Such IFNA2 fusion proteins can deliver cytokines to a desired cell type while minimizing peripheral exposure and thus reducing overall potential adverse effects.

The isolated fusion proteins include: 1) an antibody comprising an Fc domain; and b) a human IFNA2 protein variant, wherein the IFNA2 is covalently linked to the Fc domain of the antibody.

In some embodiments, one or more polypeptides (e.g., heterologous or homologous sequence) can be inserted between the antibody and the IFNA2 variant of the IFNA2 fusion proteins as described herein. In some embodiments, the polypeptide can be inserted or conjugated at the amino terminus, at the carboxyl terminus, or both the amino and carboxyl termini of the antibody or domain thereof. In some embodiments, the polypeptide includes a polypeptide linker conjugating the antibody and the IFNA2 variant. In some embodiments, the polypeptide comprises one or more linker(s) and tag(s). Examples of a polypeptide tag include, but not are not limited to, a FLAG tag, a 6His tag, an 8His tag, or an AVI tag.

The antibodies useful in the IFNA2 fusion proteins of the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies.

In some embodiments, an antibody constant region can be modified to avoid interaction with Fc gamma receptors and/or the complement system. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In still other embodiments, the constant region is a glycosylated for N-linked glycosylation, e.g., the antibodies are engineered to bypass glycosylation. In some embodiments, the constant region is a glycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is enzymatically aglycosylated for N-linked glycosylation (such as removing carbohydrate by the enzyme PNGase), or by expression in a glycosylation deficient host cell.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding to the target molecule without triggering significant complement dependent lysis, or cell-mediated destruction of the target. In some embodiments, the effector domain is capable of specifically binding to FcRn and/or FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

The antibodies used in the IFNA2 fusion proteins of the compositions and methods disclosed herein, listed by International Nonproprietary Name, can include but are not limited to abagovomab, abatacept, abciximababelacimab, abituzumab, abrezekimab, abrilumab, acapatamab, acasunlimab, acazicolcept, acrixolimab, actoxumab, adalimumab, adebrelimab, adecatumumab, adintrevimab, aducanumab, afasevikumab, afelimomab, aflibercept, alacizumab, alefacept, alemtuzumab, alirocumab, alnuctamab, alomfilimab, alsevalimab, altumomab, amatuximab, amivantamab, amlitelimab, amubarvimab, anatumomab, anbenitamab, andecaliximab, anetumab, anifrolumab, anivovetmab, anrukinzumab, anselamimab, ansuvimab, anumigilimab, apamistamab, apitegromab, apolizumab, aprutumab, arcitumomab, ascrinvacumab, aselizumab, asfotase, astegolimab, asunercept, atacicept, atezolizumab, atibuclimab, atidortoxumab, atinumab, atoltivimab, atorolimumab, avdoralimab, avelumab, avizakimab, axatilimab, azintuxizumab, bafisontamab, balstilimab, baminercept, bamlanivimab, bapincuzumab, bapotulimab, barecetamab, barzolvolimab, basiliximab, batiraxcept, batoclimab, bavituximab, bavunalimab, bebtelovimab, bectumomab, bedinvetmab, befovacimab, begelomab, belantamab, belatacept, belimumab, beludavimab, bemarituzumab, benralizumab, bentracimab, benufutamab, bepranemab, berlimatoxumab, bermekimab, bersanlimab, bertilimumab, besilesomab, betifisolimab, bevacizumab, bexmarilimab, bezlotoxumab, biciromab, bifikafusp, bimagrumab, bimekizumab, bintrafusp, birtamimab, bivatuzumab, bleselumab, blinatumomab, blisibimod, blontuvetmab, blosozumab, bococizumab, botensilimab, brazikumab, brentuximab, briakinumab, briobacept, briquilimab, brodalumab, brolucizumab, brontictuzumab, budigalimab, burfiralimab, burosumab, cabiralizumab, cadonilimab, camidanlumab, camoteskimab, camrelizumab, canakinumab, cantuzumab, caplacizumab, capromab, carlumab, carotuximab, casirivimab, catumaxomab, cedelizumab, cemiplimab, cendakimab, cergutuzumab, certolizumab, cetrelimab, cetuximab, cevostamab, cibisatamab, cifurtilimab, cilgavimab, cinpanemab, cinrebafusp, cirevetmab, citatuzumab, cixutumumab, clazakizumab, clenoliximab, clervonafusp, clesrovimab, clivatuzumab, cobolimab, codrituzumab, cofetuzumab, coltuximab, conatumumab, conbercept, concizumab, coprelotamab, cosfroviximab, cosibelimab, crefmirlimab, crenezumab, crexavibart, crizanlizumab, crotedumab, crovalimab, cudarolimab, cusatuzumab, dacetuzumab, daclizumab, dafsolimab, dalantercept, dalotuzumab, dalutrafusp, dapirolizumab, daratumumab, datopotamab, davoceticept, daxdilimab, dectrekumab, demcizumab, demupitamab, denintuzumab, denosumab, depatuxizumab, depemokimab, derlotuximab, detumomab, dezamizumab, dilpacimab, dinutuximab, diridavumab, disitamab, divozilimab, docaravimab, domagrozumab, domvanalimab, donanemab, dorlimomab, dostarlimab, dovanvetmab, dresbuxclimab, drozitumab, dulaglutide, duligotuzumab, dupilumab, durvalumab, dusigitumab, duvortuxizumab, ebdarokimab, eblasakimab, ebronucimab, ecleralimab, ecromeximab, eculizumab, edobacomab, edrecolomab, cfalizumab, efanesoctocog, efaprinermin, efavaleukin, efbemalenograstim, efdamrofusp, efepoetin, cfgartigimod, efgivanermin, efineptakin, efinopegdutide, efizonerimod, eflapegrastim, eflenograstim, eflepedocokin, eflimrufusp, efmarodocokin, efmitermant, efmoroctocog, efocipegtrutide, efpeglenatid, efpegsomatropin, efprezimod, efrilacedase, efruxifermin, eftansomatropin, eftilagimod, eftozanermin, eftrenonacog, efungumab, efzofitimod, eldelumab, elezanumab, elgemtumab, clipovimab, clotuzumab, elranatamab, elsilimomab, cluvixtamab, emactuzumab, emapalumab, emerfetamab, emfizatamab, emibetuzumab, emicizumab, emirodatamab, enapotamab, enavatuzumab, encelimab, enfortumab, enibarcimab, enlimomab, enoblituzumab, enokizumab, enoticumab, ensituximab, ensomafusp, enuzovimab, envafolimab, epcoritamab, epitumomab, epratuzumab, eptinezumab, eramkafusp, erenumab, erfonrilimab, erlizumab, ertumaxomab, etanercept, etaracizumab, etesevimab, etevritamab, etigilimab, ctokimab, ctrolizumab, evinacumab, evolocumab, evorpacept, exbivirumab, exidavnemab, czabenlimab, fanolesomab, faralimomab, faricimab, farletuzumab, fasinumab, favezelimab, fazpilodemab, feladilimab, felvizumab, felzartamab, fezakinumab, fianlimab, ficlatuzumab, fidasimtamab, figitumumab, finotonlimab, firivumab, fiztasovimab, flanvotumab, fletikumab, flotetuzumab, fontolizumab, foralumab, foravirumab, fremanezumab, fresolimumab, frexalimab, frovocimab, frunevetmab, fulranumab, futuximab, galcanezumab, galegenimab, galiximab, gancotamab, ganitumab, gantenerumab, garadacimab, garetosmab, garivulimab, gatipotuzumab, gatralimab, gavilimomab, gedivumab, gefurulimab, gemtuzumab, geptanolimab, gevokizumab, giloralimab, gilvetmab, gimsilumab, ginisortamab, girentuximab, glembatumumab, glenzocimab, glofitamab, goflikicept, golimumab, golocdacimab, gontivimab, gosuranemab, gremubamab, gresonitamab, grisnilimab, gumokimab, guselkumab, ianalumab, ibalizumab, ibritumomab, icrucumab, idactamab, idarucizumab, icramilimab, ifabotuzumab, ifinatamab, igovomab, iladatuzumab, imalumab, imaprelimab, imciromab, imdevimab, imgatuzumab, imsidolimab, imvotamab, inbakicept, inclacumab, indatuximab, indusatumab, inebilizumab, inczetamab, infliximab, inolimomab, inotuzumab, intetumumab, ipafricept, iparomlimab, ipilimumab, iratumumab, isatuximab, iscalimab, isecarosmab, ispectamab, istiratumab, itepekimab, itolizumab, ivicentamab, ivonescimab, ivuxolimab, ixekizumab, izalontamab, izenivetmab, izuralimab, keliximab, labetuzumab, lacnotuzumab, lacutamab, ladiratuzumab, lampalizumab, lanadelumab, landogrozumab, laprituximab, larcaviximab, latikafusp, latozinemab, lebrikizumab, lecanemab, lemalesomab, lemzoparlimab, lenercept, lenvervimab, lenzilumab, lepunafusp, lerdelimumab, leronlimab, lesabelimab, lesofavumab, letaplimab, letolizumab, levilimab, lexatumumab, libivirumab, licaminlimab, lifastuzumab, ligelizumab, ligufalimab, lilotomab, lintuzumab, linvoseltamab, lirentelimab, lirilumab, litifilimab, livmoniplimab, lodapolimab, lodelcizumab, lokivetmab, lomtegovimab, loncastuximab, lonigutamab, lorigerlimab, lorukafusp, lorvotuzumab, losatuxizumab, lucatumumab, lulizumab, lumiliximab, lumretuzumab, lupartumab, luspatercept, lusvertikimab, lutikizumab, luveltamab, maftivimab, magrolimab, manclimab, manfidokimab, mapatumumab, margetuximab, marstacimab, masavibart, maslimomab, matuzumab, mavrilimumab, mazorelvimab, mecbotamab, melredableukin, melrilimab, mepolizumab, metelimumab, mezagitamab, mibavademab, milatuzumab, minretumomab, mipasetamab, miptenalimab, mirikizumab, miromavimab, mirvetuximab, mirzotamab, mitazalimab, mitumomab, modakafusp, modotuximab, mogamulizumab, monalizumab, morolimumab, mosunetuzumab, motavizumab, moxetumomab, mupadolimab, murlentamab, muromonab, nacolomab, nadecnemab, nadunolimab, namilumab, naptumomab, naratuximab, narnatumab, narsoplimab, natalizumab, navicixizumab, navivumab, naxitamab, nebacumab, necitumumab, nemolizumab, nepuvibart, nerelimomab, nesvacumab, netakimab, nimacimab, nimotuzumab, nipocalimab, nirsevimab, nivatrotamab, nivolumab, nofazinlimab, nofetumomab, nurulimab, obexelimab, obiltoxaximab, obinutuzumab, obrindatamab, ocaratuzumab, ociperlimab, ocrelizumab, odesivimab, odronextamab, odulimomab, ofatumumab, ogalvibart, olamkicept, olaratumab, oleclumab, olendalizumab, olinvacimab, olokizumab, omalizumab, omburtamab, omodenbamab, onartuzumab, onfekafusp, ongericimab, ontamalimab, ontorpacept, ontuxizumab, onvatilimab, opicinumab, opinercept, oportuzumab, opucolimab, ordesekimab, oregovomab, orilanolimab, ormutivimab, orticumab, osemitamab, osocimab, otelixizumab, otilimab, otlertuzumab, oxclumab, ozanezumab, ozoralizumab, ozuriftamab, pabinafusp, pacanalotamab, pacmilimab, pagibaximab, palivizumab, pamrevlumab, panitumumab, panobacumab, paridiprubart, parsatuzumab, pascolizumab, pasotuxizumab, pateclizumab, patritumab, pavurutamab, pelgifatamab, pembrolizumab, penpulimab, pepinemab, perakizumab, peresolimab, pertuzumab, petosemtamab, pexelizumab, pidilizumab, pimivalimab, pimurutamab, pinatuzumab, pintumomab, pivekimab, placulumab, plamotamab, plonmarlimab, plozalizumab, plutavimab, polatuzumab, ponezumab, ponsegromab, porgaviximab, posdinemab, pozelimab, praluzatamab, prasinezumab, prezalumab, priliximab, pritoxaximab, pritumumab, prolgolimab, pucotenlimab, pulocimab, quavonlimab, quetmolimab, quilizumab, quisovalimab, racotumomab, radretumab, rafivirumab, ragifilimab, ralpancizumab, ramatercept, ramucirumab, ranevetmab, ranibizumab, ravagalimab, ravulizumab, raxibacumab, recaticimab, refanczumab, regavirumab, regdanvimab, relatlimab, relfovetmab, remtolumab, reozalimab, reslizumab, retifanlimab, retlirafusp, revdofilimab, rilonacept, rilotumumab, rimteravimab, rinucumab, ripertamab, risankizumab, rituximab, rivabazumab, robatumumab, rocatinlimab, roledumab, rolinsatamab, romilkimab, romlusevimab, romosozumab, rontalizumab, rosmantuzumab, rosnilimab, rosopatamab, rovalpituzumab, rovelizumab, rozanolixizumab, rozibafusp, rulonilimab, runimotamab, ruplizumab, sabatolimab, sacituzumab, samalizumab, samrotamab, sarilumab, sasanlimab, satralizumab, satumomab, secukinumab, selicrelumab, semorinemab, semzuvolimab, serclutamab, seribantumab, serplulimab, setoxaximab, setrusumab, sevirumab, sibeprenlimab, sibrotuzumab, sifalimumab, siltuximab, simlukafusp, simridarlimab, simtuzumab, sintilimab, siplizumab, sirexatamab, sirtratumab, sirukumab, socazolimab, sofituzumab, solanezumab, solitomab, sonelokimab, sontuzumab, sotatercept, sotevtamab, sotigalimab, sotrovimab, sozinibercept, spartalizumab, spesolimab, stamulumab, suciraslimab, sudubrilimab, sugemalimab, sulesomab, suptavumab, surzebiclimab, sutimlimab, suvizumab, suvratoxumab, tabalumab, tabituximab, tacatuzumab, tadocizumab, tafasitamab, tafolecimab, tagitanlimab, talacotuzumab, talizumab, talquetamab, tamgiblimab, tamrintamab, tamtuvetmab, tanezumab, taplitumomab, tarcocimab, tarextumab, tarlatamab, tavolimab, tebentafusp, tebotelimab, tecaginlimab, teclistamab, tefibazumab, telazorlimab, telimomab, telisotuzumab, telitacicept, temelimab, tenatumomab, teneliximab, teplizumab, tepoditamab, teprotumumab, teropavimab, tesidolumab, tesnatilimab, tezepelumab, tibulizumab, tidutamab, tifcemalimab, tigatuzumab, tilavonemab, tildrakizumab, tilogotamab, tilvestamab, timigutuzumab, timolumab, tinurilimab, tiragolumab, tirnovetmab, tislelizumab, tisotumab, tixagevimab, tocilizumab, tomaralimab, tomuzotuximab, toralizumab, toripalimab, torudokimab, tosatoxumab, tositumomab, tovetumab, tozorakimab, tralokinumab, trastuzumab, trebananib, tregalizumab, tremelimumab, trinbelimab, trontinemab, tucotuzumab, tulinercept, tusamitamab, tuvirumab, tuvonralimab, ubamatamab, ublituximab, ulenistamab, uliledlimab, ulocuplumab, unasnemab, upanovimab, upifitamab, urabrelimab, urelumab, urtoxazumab, ustekinumab, utomilumab, vadastuximab, valanafusp, vandortuzumab, vantictumab, vanucizumab, vapaliximab, varisacumab, varlilumab, vatelizumab, vedolizumab, veltuzumab, vepalimomab, vepsitamab, vesencumab, vibecotamab, vibostolimab, vilobelimab, visilizumab, visugromab, vixarelimab, vixtimotamab, vobarilizumab, vobramitamab, vofatamab, volagidemab, volociximab, vonlerolizumab, vopratelimab, vorsetuzumab, votumumab, voxalatamab, vudalimab, vulinacimab, vunakizumab, xeligekimab, xentuzumab, zagotenemab, zalifrelimab, zalutumumab, zamerovimab, zampilimab, zanidatamab, zanolimumab, zansecimab, zelminemab, zeluvalimab, zenocutuzumab, zilovertamab, ziltivekimab, zimberelimab, zinlirvimab, ziralimumab, zolbetuximab, zolimomab, and zuberitamab.

In some embodiments, the antibody or binding fragment thereof used in the IFNA2 fusion protein of the compositions and methods disclosed herein is an effectorless antibody or binding fragment thereof that has an isotype selected from the group consisting of NG, DANG, LALA, and LALA-PG.

In some embodiments, the antibody or binding fragment thereof used in the IFNA2 fusion protein of the compositions and methods disclosed herein, listed by International Nonproprietary Name, is an antibody selected from the group consisting of crefmirlimab, vibostolimab, and tifcemalumab, or a binding fragment thereof.

In some embodiments, the antibody or binding fragment thereof used in the IFNA2 fusion protein of the compositions and methods disclosed herein, is an antibody or binding fragment thereof that binds an antigen selected from the group consisting of human CD8A, human TIGIT, and human BTLA.

The antibody or binding fragment thereof used in the IFNA2 fusion protein of the compositions and methods disclosed herein can comprise an amino acid sequence 80%, 90%, 95%, 96%, 97%, 98%, 99%, or 100% identical to amino acid sequences selected from the group consisting of SEQ ID NO: 4, 5, 6, 7, 8, or 9:

(SEQ ID NO: 4)
EVQLVESGGGLVQPGGSLRLSCAASGFNIKDTYIHFVRQAPGKGLEWIGR
IDPANDNTLYASKFQGKATISADTSKNTAYLQMNSLRAEDTAVYYCGRGY
GYYVFDHWGQGTLVTVSS
(SEQ ID NO: 5)
DVQITQSPSSLSASVGDRVTITCRTSRSISQYLAWYQQKPGKVPKLLIYS
GSTLQSGVPSRFSGSGSGTDFTLTISSLQPEDVATYYCQQHNENPLTFGG
GTKVEIK
(SEQ ID NO: 6)
EVQLVQSGAEVKKPGSSVKVSCKASGYTFSSYVMHWVRQAPGQGLEWIGY
IDPYNDGAKYAQKFQGRVTLTSDKSTSTAYMELSSLRSEDTAVYYCARGG
PYGWYFDVWGQGTTVTVSS
(SEQ ID NO: 7)
DIQMTQSPSSLSASVGDRVTITCRASEHIYSYLSWYQQKPGKVPKLLIYN
AKTLAEGVPSRFSGSGSGTDFTLTISSLQPEDVATYYCQHHFGSPLTFGQ
GTRLEIK
(SEQ ID NO: 8)
QVQLVQSGAEVKKPGASVKLSCKASGYNFKHTYAHWVRQAPGQGLEWIGR
IDPANGNTKYDPKFQGRATMTADTASNTAYLELSSLRSEDTAVYYCVADH
YGSSLLDYWGQGTLVTVSS
(SEQ ID NO: 9)
DVVMTQTPLSLSVTPGQPASISCKSSQSLLDSDGKTYLNWFQQRPGQSPR
RLIYLVSKLDSGVPDRFSGSGSGTDFTLKISRVEAEDVGVYYCWQGTYFP
YTFGQGTKLEIK

The IFNA2 fusion proteins as described herein can be created by methods known in the art, for example, synthetically or recombinantly. Typically, the fusion proteins are made by preparing and expressing a polynucleotide encoding them using recombinant methods described herein, although they may also be prepared by other means known in the art, including, for example, chemical synthesis.

The antibodies as described herein can be made by any method known in the art. For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including, but not limited to, X63-Ag8.653 and those from the Salk Institute, Cell Distribution Center, San Diego, Calif., USA, can be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBY immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that can be used as a source of antibodies include all derivatives, and progeny cells, of the parent hybridomas that produce monoclonal antibodies.

Hybridomas that produce antibodies used for the present invention may be grown in vitro or in vivo using known procedures. The monoclonal antibodies may be isolated from the culture media or body fluids by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target, a human target protein, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N═C═NR, where R and R1 are different alkyl groups, can yield a population of antibodies (e.g., monoclonal antibodies).

If desired, the antibody (monoclonal or polyclonal) of interest can be sequenced and the polynucleotide sequence may then be cloned into a vector for expression or propagation. The sequence encoding the antibody of interest can be maintained in a vector in a host cell and the host cell can then be expanded and frozen for future use. Production of recombinant monoclonal antibodies in cell culture can be carried out through cloning of antibody genes from B cells by means known in the art. Sec, e.g. Tiller et al., J. Immunol. Methods 329, 112, 2008; U.S. Pat. No. 7,314,622.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416).

Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracyclineregulated expression of ˜(1,4)-N-acetylglucosaminyltransferase III (GnTIII), a glycosyltransferase catalyzing formation of bisecting GicNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except praline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the tripeptide sequences (for N-linked glycosylation sites) described above. The alteration may also be made by the addition of, or substitution by, one or more serine or threonine residues to the sequence of the original antibody (for O-linked glycosylation sites).

The glycosylation pattern of antibodies can also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like.

Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F 1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

The IFNA2 fusion proteins and/or the IFNA2 variants of this invention may be linked to a labeling agent such as a fluorescent molecule, a radioactive molecule, or any other labels known in the art. Labels are known in the art which generally provide (either directly or indirectly) a signal.

In some embodiments, the IFNA2 fusion protein includes an antibody including or consisting of an Fc domain and an IFNA2 variant having one or more specific substitutions at one or more of positions H30, S31, L32, S34, R35, R36, L38, L40, L41, A42, Q43, M44, R45, R46, I47, S48, L49, F50, S51, L53, K54, D55, R56, H57, D58, F59, F61, P62, Q63, Q69, K72, V78, M82, Q84, I86, K93, A98, L103, K106, Y108, T109, E110, Q113, N116, N116, E119, A120, G125, V126, T129, P132, M134, I139, A141, R143, Y145, Q147, R148, E155, K156, K157, P160, V165, R167, A168, E169, 1170, M171, R172, S173, S175, L176, S177, N179, S183, R185, S186, K187, and E188 in the wild-type IFNA2 amino acid sequence (SEQ ID NO: 1).

In some embodiments, the IFNA2 fusion protein includes an antibody comprising a Fc domain and an IFNA2 variant comprising one or more specific substitutions selected from the group consisting of: H30A, H30D, S31D, L32D, L32E, S34A, R35N, R36G, L38H, L38Y, L38G, L38P, L38S, L38T, L38Q, L38N, L40R, L40G, L40Q, L40D, L41H, L41D, L41K, L41G, L41A, A42G, A42M, Q43P, M44Q, R45P, R46P, R46D, R46F, R46Y, R46N, R46S, R461, R46G, R46A, R46H, R46T, 147P, 147D, 147S, 147E, S48Y, S48H, L49H, L491, L49K, L49V, L49Y, L49F, L49G, L49E, L49P, F50D, F50N, F50G, F50Q, F50S, F50M, F50H, F50A, S51D, S51E, L53D, L53E, L53G, L53A, L53N, L53V, L53S, K54L, K54I, K54M, D55T, D55N, D55Q, R56D, R56G, R56K, R56N, R56V, R56T, R56A, R56L, R56H, H57P, D58Q, F59Q, F59E, F591, F59N, F59A, F59G, F59T, F59Y, F61G, F611, F61V, F61P, F61Q, F61A, F61S, P621, Q63E, Q69S, K72D, V78E, V78G, M82W, M82K, M82R, M82G, M82S, 186L, K93E, K93V, A98E, L103E, L103D, K106L, K106D, Y108K, T109W, E110P, E110S, Q113W, N116W, N116M, N116F, N116Q, E119Y, E119F, A120M, G125V, V126W, V126Y, T129G, P132W, M134Y, I139Y, I139F, A141E, A141I, R143F, Q147S, R148Y, E155H, K156D, K156L, K156W, K157Y, P160E, P160F, P160W, P160Y, P160T, V165Y, V165E, V165H, V165K, V165W, V165F, V165Q, V165L, V165M, V165S, V165R, V165N, V165D, V165I, R167W, R167I, R167M, R167S, R167E, R167L, R167V, R167A, R167G, R167H, A168R, A168H, A168K, A168Y, A168G, A168F, A168D, A168M, A168Q, E169T, E169S, E169G, 1170L, M171R, M171S, M171T, M171N, M171A, M171Y, M171W, M171F, M171K, M171E, M171G, M171L, M171I, M171V, R172D, R172W, R172N, R172A, R172V, R172G, R172T, R172S, R172Y, R172L, R172M, R172K, S173K, S173R, S173H, S173E, S173N, S173W, S173Y, S175R, S175K, S175M, S175L, S175P, S175I, S175V, S175W, S175Y, S175G, S175E, S175Q, S175T, L176N, L176G, L176H, L176A, L176P, L176D, L176R, L176Q, L176E, L176V, S177D, S177R, N179G, S183W, S183F, S183M, S183E, S183Y, S183K, S183L, R185D, R185E, R185Q, S186L, S186I, S186D, K187R, K187L, K187E, K187Y, K187V, K187M, K187F, K187I, K187W, K187G, and E1881 in the wild-type IFNA2 amino acid sequence (SEQ ID NO: 1).

In some embodiments, the IFNA2 fusion protein includes an antibody comprising an Fc domain and an IFNA2 variant including a combination of two specific amino acid substitutions, i.e., a double amino acid substitution, selected from the group consisting of: M44N+A168R, L49P+S175P, L49P+S173H, L49P+M171W, L49P+M171G, L49P+S173R, M44N+S175P, L49P+M171V, L49P+M171K, L49P+M171S, L49P+S175R, L49P+M171R, L49P+L176G, L49P+M171Y, L49P+L176N, L49P+S173Q, L49P+L176H, L49P+L176R, L49P+M171A, L49P+M171T, L49P+L176A, L49P+M171F, M44N+S175K, L49P+S175K, L49P+L176E, L49P+E169T, L49P+M171L, L49P+L176Q, L49P+K187E, L49P+S173N, M44N+L176H, M44N+S175R, L49P+S177N, L49P+S173W, L49P+S177D, L49P+R185D, L49P+E169S, L49P+S175G, L49P+S177L, L49P+R172K, L49P+R185E, L49P+K187M, L49P+K187W, L49P+S159Q, M44N+L176Q, M44N+R185E, L49P+S175Q, L49P+S175M, L49P+E155V, L49P+K187D, L49P+S175W, L49P+L176V, L49P+S173L, M44N+S175G, L49P+R185G, L49P+R143W, L49P+1149T, L49P+R185L, L49P+R185S, L49P+S177H, L49P+K106D, M44N+R185N, L49P+K187V, L49P+K106W, L49P+K135T, M44N+V1281, M44N+R185D, L49P+K106P, L49P+S183M, L49P+S183D, L49P+K156A, L49P+S183L, M44N+K187D, L49P+R185M, L49P+R185N, M44N+A168L, L49P+S175L, L49P+R185Q, L49P+K187G, M44N+S177E, L49P+W163L, L49P+R185W, L49P+K187T, L49P+T109W, M44N+P160E, M44N+K106T, L49P+R185T, L49P+S175I, L49P+S173F, M44N+R185L, L49P+S183E, L49P+K187N, M44N+K187F, L49P+S159D, M44N+K106I, L49P+S175Y, L49P+L184N, L49P+I170L, L49P+V166S, L49P+K187A, L49P+S186D, L49P+L111F, L49P+K154Q, L49P+K106T, L49P+E136D, L49P+K106M, L49P+F107W, M44N+R185Y, M44N+V165I, M44N+K187W, L49P+R185I, M44N+R185T, M44N+R185V, M44N+Q113A, M44N+T178K, L49P+K156V, L49P+K187S, M44N+Q114I, L49P+T131E, M44N+S183E, L49P+Y152W, L49P+V128E, L49P+E155K, L49P+S175T, L49P+E155R, L49P+V166T, and L49P+T178F in the wild-type IFNA2 amino acid sequence (SEQ ID NO: 1).

In some embodiments, the IFNA2 fusion protein has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

### Polynucleotides, Vectors, Host Cells

The disclosure also provides polynucleotides encoding any of the IFNA2 variants and IFNA2 fusion proteins as described herein. In one aspect, the disclosure provides methods of making any of the polynucleotides described herein. Polynucleotides can be made and expressed by procedures known in the art.

In another aspect, the disclosure provides compositions (such as pharmaceutical compositions) including any of the polynucleotides described herein. In some embodiments, the composition includes an expression vector having a polynucleotide encoding any of the IFNA2 variants and IFNA2 fusion proteins described herein.

In another aspect, the disclosure provides isolated cell lines that produce the IFNA2 variants and the IFNA2 fusion proteins as described herein. In some embodiments, the cell line is an engineered immune cell, wherein the engineered immune cell includes a chimeric antigen receptor (CAR). In some embodiments, the IFNA2 variants and the IFNA2 fusion proteins, when expressed as polynucleotides in CAR T cells, either as secreted or membrane-tethered versions, are used to enhance CAR T function, including activity and proliferation.

Immune cells producing the IFNA2 variants and the IFNA2 fusion proteins as described herein may be made by introducing a CAR into immune cells, and expanding the cells. For example, the immune cells can be engineered by providing a cell and expressing at the surface of the cell at least one CAR and at least one IFNA2 variant or IFNA2 fusion protein as described herein. Methods for engineering immune cells are described in, for example, PCT Patent Application Publication Nos. WO/2014/039523, WO/2014/184741, WO/2014/191128, WO/2014/184744, and WO/2014/184143, each of which is incorporated herein by reference in its entirety. In some embodiments, the cell can be transformed with at least one polynucleotide encoding a CAR, one polynucleotide encoding the IFNA2 variant or IFNA2 fusion protein as described herein, followed by expressing the polynucleotides in the cell.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide described herein, and a polynucleotide may, but need not, be linked to other molecules and/or support materials. Polynucleotides can include a native sequence (e.g., an endogenous sequence that encodes an antibody or a fragment thereof) or may include a variant of such a sequence. Polynucleotide variants of antibodies or fragments thereof contain one or more substitutions, additions, deletions, and/or insertions such that the immunoreactivity of the encoded polypeptide is not diminished, relative to a native immunoreactive molecule. The effect on the immunoreactivity of the encoded polypeptide may generally be assessed as described herein and by methods known in the relevant field. Variants of antibodies or fragments thereof can exhibit at least about 70% identity, at least about 80% identity, at least about 90% identity, or at least about 95, 96, 97, 98, or 99% identity to a polynucleotide sequence that encodes a native antibody or a fragment thereof.

Variants are substantially homologous to a native gene, or a portion or complement thereof. It will be appreciated by those of ordinary skill in the art that, as a result of the degeneracy of the genetic code, there are many nucleotide sequences that encode polypeptides as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide having a desired sequence can be inserted into a suitable vector, and the vector in turn can be introduced into a suitable host cell for replication and amplification, as further discussed herein. Polynucleotides can be inserted into host cells by any means known in the art. Cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

As is well known, PCR allows reproduction of DNA sequences. See, e.g. U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors can be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp 18, mp 19, pBR322, pMB9, ColEl, pCRI, RP4, phage DNAs, and shuttle vectors such as pSA3 and pAT28. These and many other cloning vectors are available from commercial vendors such as BioRad, Strategene, and Invitrogen.

Expression vectors are also provided herein. Expression vectors generally are replicable polynucleotide constructs that contain a polynucleotide as described herein. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. The disclosure also provides host cells including any of the polynucleotides described herein. Any host cells capable of over-expressing heterologous DNAs can be used for the purpose of isolating the genes encoding the antibody, polypeptide, or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisae, S. pombe; or K. lactis). Preferably, the host cells express the cDNAs at a level of about 5 fold higher, 10 fold higher, or 20 fold higher than that of the corresponding endogenous antibody or protein of interest, if present, in the host cells. Screening the host cells for a specific binding to IFNA2 or a IFNA2 domain can be effected by an immunoassay or FACS. A cell overexpressing the antibody or protein of interest can be identified.

An expression vector can be used to direct expression of an IFNA2 variant or an IFNA2 fusion protein. One skilled in the art is familiar with administration of expression vectors to obtain expression of an exogenous protein in vivo. Sec, e.g., U.S. Pat. Nos. 6,436,908; 6,413,942; and 6,376,471. Administration of expression vectors includes local or systemic administration, including injection, oral administration, particle gun, catheterized administration, and topical administration. In another embodiment, the expression vector is administered directly to the sympathetic trunk or ganglion, or into a coronary artery, atrium, ventricle, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J. A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338.

Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 pg to about 500 pg, and about 20 pg to about 100 pg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura, Human Gene Therapy, 1994, 5:845; Connelly, Human Gene Therapy, 1995, 1:185; and Kaplitt, Nature Genetics, 1994, 6:148). Expression of such coding sequences can be induced using endogenous mammalian or heterologous promoters. Expression of the coding sequence can be either constitutive or regulated. Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936: WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3: 147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264: 16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581.

### Compositions

The disclosure also provides pharmaceutical compositions including an effective amount of an IFNA2 variant or an IFNA2 fusion protein as described herein. Examples of such compositions, as well as how to formulate, are also described herein. In some embodiments, the composition includes one or more IFNA2 variants, combinations with other variant detuned cytokines or other IFNA2 fusion proteins.

In some embodiments, the compositions include an IFNA2 fusion protein including an antibody and a human IFNA2 variant having one or more, e.g., two specific, or more, specific substitutions in SEQ ID NO: 1 as described herein.

It is understood that the compositions can include more than one IFNA2 variant or IFNA2 fusion protein (e.g., a mixture of IFNA2 variants or IFNA2 fusion proteins comprising different IFNA2 variants and/or different antibodies).

The compositions disclosed herein can further include pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at known dosages and concentrations, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG). Pharmaceutically acceptable excipients are further described herein.

The IFNA2 variants, IFNA2 fusion proteins, and compositions thereof can also be used in conjunction with, or administered separately, simultaneously, or sequentially with other agents that serve to enhance and/or complement the effectiveness of the agents.

The disclosure also provides compositions, including pharmaceutical compositions, including any of the polynucleotides described herein. In some embodiments, the compositions include an expression vector including a polynucleotide encoding the IFNA2 variants and IFNA2 fusion proteins as described herein. In other embodiments, the compositions include an expression vector having a polynucleotide encoding any of the IFNA2 variants and IFNA2 fusion proteins described herein.

### Methods of Treatment

The IFNA2 variants and the IFNA2 fusion proteins described herein are useful in various applications including, but are not limited to, therapeutic treatment methods and diagnostic treatment methods.

In one aspect, the disclosure provides methods for treating cancer. In some embodiments, the methods of treating cancer in a subject include administering to the subject in need thereof an effective amount of a composition (e.g., a pharmaceutical composition) including any of the IFNA2 variants and the IFNA2 fusion proteins as described herein. As used herein, a cancer can be a solid cancer or a blood or bone marrow cancer. Solid cancers include, but are not limited to, gastric cancer, small intestine cancer, sarcoma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer, mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colorectal cancer, oral cancer, skin cancer, Merkel cell carcinoma, glioblastoma, brain tumor, bone cancer, eye cancer, and melanoma.

Blood and bone marrow cancers include, but are not limited to, multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis, T cell histiocyte-rich large B-cell lymphoma, primary central nervous system lymphoma, primary cutaneous diffuse large B-cell lymphoma (leg type), EBY positive diffuse large B-cell lymphoma of the elderly, diffuse large B-cell lymphoma associated with inflammation, intravascular large B-cell lymphoma, ALK-positive large B-cell lymphoma, plasmablastic lymphoma, large B-cell lymphoma arising in HHVS-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other hematopoietic cell related cancer. In some embodiments, the cancer is relapsed, refractory, or metastatic.

In some embodiments, the methods of inhibiting tumor growth or progression in a subject include administering to the subject in need thereof an effective amount of a composition including the IFNA2 variants or IFNA2 fusion proteins as described herein. In some embodiments, the disclosure includes methods of inhibiting metastasis of cancer cells in a subject, which include administering to a subject in need thereof an effective amount of a composition including any of the IFNA2 variants or IL-fusion proteins as described herein. In other embodiments, the disclosure includes methods of inducing regression of a tumor in a subject, which include administering to the subject in need thereof an effective amount of a composition including any of the IFNA2 variants or IFNA2 fusion proteins as described herein. In another aspect, the disclosure provides methods of detecting, diagnosing, and/or monitoring a cancer. For example, the IFNA2 variants or IFNA2 fusion proteins as described herein can be labeled with a detectable moiety such as an imaging agent and an enzyme-substrate label. The IFNA2 variants or IFNA2 fusion proteins as described herein can also be used for in vivo diagnostic assays, such as in vivo imaging (e.g., PET or SPECT), or a staining reagent.

In some embodiments, the methods described herein further include a step of treating a subject with an additional form of therapy. In some embodiments, the additional form of therapy is an additional anti-cancer therapy including, but not limited to, chemotherapy, radiation, surgery, hormone therapy, checkpoint inhibitor and/or additional immunotherapy.

With respect to all methods described herein, reference to IFNA2 variants or IFNA2 fusion proteins also includes compositions comprising one or more additional agents. These compositions may further comprise suitable excipients, such as pharmaceutically acceptable excipients including buffers, which are well known in the art. The methods and compositions described herein can be used alone or in combination with other methods of treatment.

The IFNA2 variants or IFNA2 fusion proteins as described herein can be administered to a subject via any suitable route. It should be apparent to a person skilled in the art that the examples described herein are not intended to be limiting, but to be illustrative of the techniques available. Accordingly, in some embodiments, the IL-variant or IFNA2 fusion protein is administered to a subject in accord with known methods, such as intravenous administration, e.g., as a bolus or by continuous infusion over a period of time, by intramuscular, intraperitoneal, intracerebrospinal, transdermal, subcutaneous, intra-articular, sublingually, or intrasynovial administration, or via insufflation, intrathecal, oral, inhalation, or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, the IFNA2 variants or IFNA2 fusion proteins can be aerosolized using a fluorocarbon formulation and a metered dose inhaler, or inhaled as a lyophilized and milled powder.

In some embodiments, an IFNA2 variant or IFNA2 fusion protein is administered via site-specific or targeted local delivery techniques. Examples of site-specific or targeted local delivery techniques include direct injection into patient tumor or various implantable depot sources of the IL-variants or IFNA2 fusion proteins or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of an IFNA2 variant or IFNA2 fusion protein can be used for administration. In some embodiments, the IFNA2 variant or IFNA2 fusion protein can be administered neat. In some embodiments, the IFNA2 variant or IFNA2 fusion protein and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000. In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

Generally, for administration of IFNA2 variants or IFNA2 fusion proteins, the candidate dosage can be administered daily, every other day, every third day, every week, every other week, every three weeks, every four weeks, every five weeks, every six weeks, every seven weeks, every eight weeks, every ten weeks, every twelve weeks, or more than every twelve weeks. For repeated administrations over several days or longer, depending on the condition, the treatment is sustained until a desired suppression of symptoms occurs or until sufficient therapeutic levels are achieved, for example, to reduce symptoms associated with cancer. The progress of this therapy is easily monitored by conventional techniques and assays. The dosing regimen (including the specific IFNA2 variants or IFNA2 fusion proteins used) can vary over time.

In some embodiments, the candidate dosage is administered daily with the dosage ranging from about any of 1 μg/kg to 30 pg/kg to 300 μg/kg to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg to 30 pg/kg to 300 μg/kg to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg to 30 pg/kg to 300 μg/kg to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg to 30 pg/kg to 300 μg/kg to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In some embodiments, the candidate dosage is administered every month or every four weeks with the dosage ranging from about any of 1 μg/kg to 30 pg/kg to 300 pg/kg to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used.

In some embodiments, a therapeutic of the present invention is administered at a dose ranging from about 1 μg/kg to about 600 μg/kg or more, about 6 μg/kg to about 600 μg/kg, about 6 μg/kg to about 300 μg/kg, about 30 μg/kg to about 600 μg/kg or about 30 μg/kg to about 300 μg/kg. For example, the dose is administered at about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 7 5 μg/kg, about 80 μg/kg, about 85 μg/kg, about 90 μg/kg, about 95 μg/kg, about 100 μg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 350 μg/kg, about 400 μg/kg, about 450 μg/kg, about 500 μg/kg, about 550 μg/kg or about 600 μg/kg may be used.

For the purposes of the present disclosure, the appropriate dosage of an IFNA2 variant or an IFNA2 fusion protein will depend on the IFNA2 variant or an IFNA2 fusion protein (or compositions thereof) employed, the type and severity of symptoms to be treated, whether the agent is administered for preventive or therapeutic purposes, previous therapy, the patient's clinical history and response to the agent, the patient's clearance rate for the administered agent, and the discretion of the attending physician. Typically the clinician will administer an IFNA2 variant or an IFNA2 fusion protein until a dosage is reached that achieves the desired result. Dose and/or frequency can vary over the course of treatment. In some embodiments, step dosing is performed where the initial dose or doses are lower than incrementally higher doses administered as the regiment continues. Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of symptoms. Alternatively, sustained continuous release formulations of IFNA2 variants or IFNA2 fusion proteins may be appropriate. Various formulations and devices for achieving sustained release are known in the art.

In one embodiment, dosages for an IFNA2 variant or an IFNA2 fusion protein may be determined empirically in individuals who have been given one or more administration (s) of an IFNA2 variant or an IFNA2 fusion protein. For example, individuals are given incremental dosages of an IFNA2 variant or an IFNA2 fusion protein. To assess efficacy, an indicator of the disease can be followed. Administration of an IFNA2 variant or an IFNA2 fusion protein as described herein in accordance with the method in the present invention can be continuous or intermittent, depending, for example, upon the recipient's physiological condition, whether the purpose of the administration is therapeutic or prophylactic, and other factors known to skilled practitioners. The administration of an IFNA2 variant or an IFNA2 fusion protein may be essentially continuous over a preselected period of time or may be in a series of spaced doses.

In some embodiments, more than one IFNA2 variant or IFNA2 fusion protein may be present. At least one, at least two, at least three, at least four, at least five, or more different IFNA2 variants or IFNA2 fusion proteins can be present. Generally, those IL-variants or IFNA2 fusion proteins may have complementary activities that do not adversely affect each other.

In some embodiments, the IFNA2 variant or the IFNA2 fusion protein may be administered in combination with one or more additional therapeutic agents. These include, but are not limited to, a biotherapeutic agent, a chemotherapeutic agent, a vaccine, a CAR-T cell-based therapy, radiotherapy, another cytokine therapy (e.g., immunostimulatory cytokines including various signaling proteins that stimulate immune response, such as interferons, interleukins, and hematopoietic growth factors), a vaccine, an inhibitor of other immunosuppressive pathways, e.g., anti-PD-1 checkpoint inhibitors, an inhibitors of angiogenesis, a T cell activator, an inhibitor of a metabolic pathway, an mTOR (mechanistic target of rapamycin) inhibitor (e.g., rapamycin, rapamycin derivatives, sirolimus, temsirolimus, everolimus, and deforolimus), an inhibitor of an adenosine pathway, a tyrosine kinase inhibitor including but not limited to inlyta, ALK (anaplastic lymphoma kinase) inhibitors (e.g., crizotinib, ceritinib, alectinib, and sunitinib), a BRAF inhibitor (e.g., vemurafenib and dabrafenib), an epigenetic modifier, an inhibitors or depletor of Treg cells and/or of myeloid-derived suppressor cells, a JAK (Janus Kinase) inhibitor (e.g., ruxolitinib and tofacitinb, varicitinib, filgotinib, gandotinib, lestaurtinib, momelotinib, pacritinib, and upadacitinib), a STAT (Signal Transducers and Activators of Transcription) inhibitor (e.g., STATI, STAT3, and STATS inhibitors such as fludarabine), a cyclin-dependent kinase inhibitor, an immunogenic agent (for example, attenuated cancerous cells, tumor antigens, antigen presenting cells such as dendritic cells pulsed with tumor derived antigen or nucleic acids, a MEK inhibitor (e.g., tramctinib, cobimetinib, binimetinib, and selumetinib), a GLSI inhibitor, a PAP inhibitor, an oncolytic virus, an IDO (Indoleamine-pyrrole 2,3-dioxygenase) inhibitor, a PRR (Pattern Recognition Receptors) agonist, and cells transfected with genes encoding immune stimulating cytokines such as but not limited to GM-CSF).

In some embodiments, examples of immunostimulatory cytokines include, but are not limited to, GM-CSF, G-CSF, IFNy, IFNa; IL-2 (e.g. denileukin difitox), IL-6, IL-7, IL-10, IL-11, IL-12, IFNA2, IL-18, IL-21, and TNFa. In some embodiments, the cytokines are pegylated (e.g., pegylated IL-2, IL-10, IFNy, and IFNa).

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein. The terms “TLR” and “toll-like receptor” refer to any toll-like receptor. Toll-like receptors are receptors involved in activating immune responses. TLRs recognize, for example, pathogen-associated molecular patterns (PAMPs) expressed in microbes, as well as endogenous damage-associated molecular patterns (DAMPs), which are released from dead or dying cells.

Molecules that activate TLRs (and thereby activate immune responses) are referred to herein as “TLR agonists.” TLR agonists can include, for example, small molecules (e.g. organic molecule having a molecular weight under about 1000 Daltons), as well as large molecules (e.g. oligonucleotides and proteins). Some TLR agonists are specific for a single type ofTLR (e.g. TLR3 or TLR9), while some TLR agonists activate two or more types of TLR (e.g. both TLR7 and TLRS).

Examples of TLR agonists provided herein include agonists of TLR2, TLR3, TLR4, TLR5, TLR6, TLR7, TLRS, and TLR9.

Examples of small molecule TLR agonists include those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; 6,818,650; and 7,7091, 214; U.S. Patent Publication Nos. 2004/0091491, 2004/0176367, and 2006/0100229; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, WO 2005/079195, WO 2005/094531, WO 2005/123079, WO 2005/123080, WO 2006/009826, WO 2006/009832, WO 2006/026760, WO 2006/028451, WO 2006/028545, WO 2006/028962, WO 2006/029115, WO 2006/038923, WO 2006/065280, WO 2006/074003, WO 2006/083440, WO 2006/086449, WO 2006/091394, WO 2006/086633, WO 2006/086634, WO 2006/091567, WO 2006/091568, WO 2006/091647, WO 2006/093514, and WO 2006/098852.

Additional examples of small molecule TLR agonists include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329, 381; and in WO 02/08905), and certain 3-.beta.-D-ribofuranosylthiazolo[4,5-d]pyrimidine derivatives (such as those described in U.S. Publication No. 2003/0199461), and certain small molecule immuno-potentiator compounds such as those described, for example, in U.S. Patent Publication No. 2005/0136065.

Examples of large molecule TLR agonists include TLR agonist oligonucleotide sequences. Some TLR agonist oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other TLR agonist nucleotide sequences lack CpG sequences and are described, for example, in International Patent Publication No. WO 00/75304. Still other TLR agonist nucleotide sequences include guanosine- and uridine-rich single-stranded RNA (ssRNA) such as those described, for example, in Heil et ah, Science, vol. 303, pp. 1526-1529 Mar. 5, 2004.

Other TLR agonists include biological molecules such as aminoalkyl glucosaminide phosphates (AGPs) and are described, for example, in U.S. Pat. Nos. 6,113,918; 6,303,347; 6,525,028; and 6,649,172.

TLR agonists also include inactivated pathogens or fractions thereof, which may activate multiple different types of TLR receptor. Exemplary pathogen-derived TLR agonists include BCG, Mycobacterium obuense extract, Talimogene laherparepvec (T-Vec) (derived from HSV-1), and Pexa-Vec (derived from vaccina virus).

In some embodiments, a TLR agonist may be an agonist antibody that binds specifically to the TLR.

In some embodiments, the biotherapeutic agent is an antibody, including but not limited to, an anti-CTLA-4 antibody, an anti-CD3 antibody, an anti-CD4 antibody, an anti-CD8 antibody, an anti-4-1BB antibody, an anti-PD-1 antibody, an anti-PD-Llantibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSFIR antibody, an anti-CSFI antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFRI antibody, an antiTNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCRS antibody, an anti-CD200R antibody, an antiVISG4 antibody, an anti-CCR2 antibody, an anti-LILRb2 antibody, an anti-CXCR4 antibody, an anti-CD206 antibody, an anti-CD163 antibody, an anti-KLRGI antibody, an antiFLT3 antibody, an anti-B7-H4 antibody, an anti-B7-H3 antibody, an KLRGI antibody, a BTNIAl antibody, a BCMA antibody, a CLEC9A antibody, a LILRB4 antibody, or an anti-GITR antibody.

In some embodiments, an IFNA2 variant or an IFNA2 fusion protein is used in combination with an immunocytokine. In some embodiments, the immunocytokine includes an antibody, or fragment thereof, conjugated or fused to a cytokine (e.g., fusion protein). In some embodiments, the antibody, or fragment thereof, binds to the Extra Domain-A (EDA) isoform of fibronectin (e.g. anti-EDA antibody). Accordingly, in some embodiments, an IFNA2 variant or an IFNA2 fusion protein is used in conjunction with, for example, an anti-PD-LI antagonist antibody; an anti-PD-I antagonist antibody such as for example, nivolumab (OPDIVO®), pembrolizumab (KEYTRUDA®), mAb7 (e.g., as described in US Pub. No. US20160i59905, hereby incorporated by reference), and pidilizumab; an anti-CTLA-4 antagonist antibody such as for example ipilimumab (YERVOY®); an anti-LAG-3 antagonist antibody such as BMS-9860i6 and IMP70i; an anti-TIM-3 antagonist antibody; an anti-17-1H3 antagonist antibody such as for example MGA27i; an-anti-VISTA antagonist antibody; an anti-TIGIT antagonist antibody; an anti-CD28 antagonist antibody; an anti-CD8 antibody; an anti-CD86 antibody; an anti-B7-H4 antagonist antibody; an anti-ICOS agonist antibody; an anti-CD28 agonist antibody; an innate immune response modulator (e.g., TLRs, KIR, NKG2A), and an IDO inhibitor. In some embodiments, an IFNA2 variant or an IFNA2 fusion protein is used in conjunction with a 4-18B (CD137) agonist such as, for example, PF-05082566 or urclumab (BMS-663513). In some embodiments, an IFNA2 variant or an IFNA2 fusion protein is used in conjunction with an OX40 agonist such as, for example, an anti-OX-40 agonist antibody. In some embodiments, an IFNA2 variant or an IFNA2 fusion protein is used in conjunction with a GITR agonist such as, for example, TRX518. In some embodiments, an IFNA2 variant or an IFNA2 fusion protein is used in conjunction with an IDO inhibitor. In some embodiments, an IFNA2 variant or an IFNA2 fusion protein is used in conjunction with a cytokine therapy such as, for example without limitation, (pegylated or non-pegylated) IL-2, IL-10, IL-12, IL-7, IL-15, IL-21, IL-33, CSF-1, MCSF-1, etc.

In some embodiments, other examples of the antibody for the combination use with the IFNA2 variant or the IFNA2 fusion protein of the present invention can be directed to, 5T4; A33; alpha-folate receptor 1 (e.g. mirvetuximab soravtansine); Alk-1; CA-125 (e.g. abagovomab); Carboanhydrase IX; CCR2; CCR4 (e.g. mogamulizumab); CCR5 (e.g. Ieronlimab); CCR8; CD3 [e.g. blinatumomab (CD3/CD19 bispecific), PF-06671008 (CD3/P-cadherin bispecific), PF-06863135 (CD3/BCMA bispecific), CD25; CD28; CD30 (e.g. brentuximab vedotin); CD33 (e.g. gemtuzumab ozogamicin); CD38 (e.g. daratumumab, isatuximab), CD44v6; CD63; CD79 (e.g. polatuzumab vedotin); CD80; CD123; CD276/B7-H3 (e.g. omburtamab); CDH17; CEA; ClhCG; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); GD2 (e.g. dinutuximab, 3F8); GD3; GloboH; GMI; GM2; HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; ITG-AvB6; LAG-3 (e.g. relatlimab); Lewis-Y; LG; Ly-6; M-CSF [e.g. PD-0360324 (see U.S. Pat. No. 7,326,414)]; MCSP; mesothelin; MUCI; MUC2; MUC3; MUC4; MUCSAC; MUCSB; MUC7; MUC16; Notchl; Notch3; Nectin-4 (e.g. enfortumab vedotin); P-Cadherein [e.g. PF-06671008 (see WO2016/001810)]; PCDHB2; PDGFRA (e.g. olaratumab); Plasma Cell Antigen; PolySA; PSCA; PSMA; PTK7 [e.g. PF-06647020 (see U.S. Pat. No. 9,409,995)]; Rorl; SAS; SCRx6; SLAMF7 (e.g. clotuzumab); SHH; SIRPa (e.g. ED9, Effi-DEM); STEAP; TGF-beta; TIGIT; TMPRSS3; TNF-alpha precursor; TROP-2 (e.g., sacituzumab govitecan); TSPAN8; and Wue-1. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurca; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; ctoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are antihormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LYIl 7018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and antiandrogens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase inhibitors such as sunitinib, axitinib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857; EGFR T790M inhibitors such as PF-06747775; EZH2 inhibitors such as PF-06821497; PRMT5 inhibitors such as PF-06939999; TGFR˜rl inhibitors such as PF-06952229; and pharmaceutically acceptable salts, acids or derivatives of any of the above.

In some embodiments, an IFNA2 variant or an IFNA2 fusion protein is used in conjunction with one or more other therapeutic agents targeting an immune checkpoint modulator, such as, for example without limitation, an agent targeting PD-1, PDL-1, CTLA-4, LAG-3, B7-H3, B7-H4, B7-DC (PD-L2), B7-H5, B7-H6, B7-H8, B7-H2, B7-1, B7-2, ICOS, ICOS-L, TIGIT, CD2, CD47, CD80, CD86, CD48, CD58, CD226, CD155, CD112, LAIR1, 2B4, BTLA, CD160, TIMI, TIM-3, TIM4, VISTA (PD-HI), OX40, OX40L, GITRL, CD70, CD27, 4-18B, 4-BBL, DR3, TLIA, CD40, CD40L, CD30, CD30L, LIGHT, HVEM, SLAM (SLAMFI, CD150), SLAMF2 (CD48), SLAMF3 (CD229), SLAMF4 (2B4, CD244), SLAMF5 (CD84), SLAMF6 (NTB-A), SLAMCF7 (CS!), SLAMF8 (BLAME), SLAMF9 (CD2F), CD28, CEACAM1 (CD66a), CEACAM3, CEACAM4, CEACAM5, CEACAM6, CEACAM7, CEACAM8, CEACAM1-3AS CEACAM3C2, CEACAM1-15, PSG1-11, CEACAM1-4CI, CEACAM1-4S, CEACAM1-4L, IDO, TDO, CCR2, CD39-CD73-adenosine pathway (A2AR), BTKs, TIKs, CXCR2, CCR4, CCR8, CCR5, VEGF pathway, CSF-1, or an innate immune response modulator.

In some embodiments, an IFNA2 variant or an IFNA2 fusion protein is used in conjunction with a biotherapeutic agent and a chemotherapeutic agent. For example, provided is a method for treating cancer in a subject in need thereof including administering to the subject an effective amount of the IFNA2 variant or IFNA2 fusion protein as described herein, a therapeutic antibody, and a chemotherapeutic agent (e.g., gemcitabine, methotrexate, or a platinum analog). In some embodiments, provided is a method for treating cancer in a subject in need thereof comprising administering to the subject an effective amount of the IFNA2 variant or IFNA2 fusion protein as described wherein, a therapeutic antibody (e.g., nivolumab (OPDIVO®), mAb7 (e.g., as described in US Pub. No. US20160159905, hereby incorporated by reference), or pembrolizumab (KEYTRUDA®), and a chemotherapeutic agent (e.g., gemcitabine, methotrexate, or a platinum analog). In some embodiments, provided is a method for treating cancer in a subject in need thereof comprising administering to the subject an effective amount of the IFNA2 variant or IFNA2 fusion protein as described wherein, an anti-CTLA-4 antagonist antibody (e.g., ipilimumab (YERVOY®)), and a chemotherapeutic agent (e.g., gemcitabine, methotrexate, or a platinum analog).

In some embodiments, the IFNA2 variant or IFNA2 fusion protein therapy may precede or follow the other agent treatment by intervals ranging from minutes to weeks. In embodiments where the other agents and/or a proteins or polynucleotides are administered separately, one would generally ensure that a significant period of time did not expire between each delivery, such that the agent and the composition of the present invention would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

In some embodiments, an IFNA2 variant or an IFNA2 fusion protein composition comprises a second agent selected from crizotinib, palbociclib, gemcitabine, cyclophosphamide, fluorouracil, FOLFOX, folinic acid, oxaliplatin, axitinib, sunitinib malate, tofacitinib, bevacizumab, rituximab, and trastuzumab.

In some embodiments, an IFNA2 variant or IFNA2 fusion protein composition is combined with a treatment regimen further comprising a traditional therapy selected from the group consisting of: surgery, radiation therapy, chemotherapy, targeted therapy, immunotherapy, hormonal therapy, angiogenesis inhibition and palliative care.

### Formulations

Therapeutic formulations of the IFNA2 variant or IFNA2 fusion protein used in accordance with the present disclosure are prepared for storage by mixing the protein having the desired degree of purity with optional pharmaceutically acceptable carriers, excipients, or stabilizers (Remington, The Science and Practice of Pharmacy 20th Ed. Mack Publishing, 2000), e.g., in the form of lyophilized formulations or aqueous solutions. Acceptable carriers, excipients, or stabilizers are nontoxic to recipients at the dosages and concentrations employed, and may include buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEWM, PLURONICS™ or polyethylene glycol (PEG).

Liposomes containing the IFNA2 variant or IFNA2 fusion protein are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The active ingredients may also be entrapped in microcapsules prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20^{th} Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, nondegradable ethylene-vinyl acetate, degradable lactic acidglycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic IFNA2 variant or IFNA2 fusion protein compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g. Span™ 20, 40, 60, 80 or 85). Compositions with a surfaceactive agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an IFNA2 variant or IFNA2 fusion protein with Intralipid™ or the components thereof (soybean oil, egg phospholipids, glycerol and water). Compositions for inhalation or insufllation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulizing device or the nebulising device may be attached to a face mask, tent or intermittent positive pressure breathing machine. Solution, suspension or powder compositions may be administered, preferably orally or nasally, from devices which deliver the formulation in an appropriate manner. Kits

The invention also provides kits comprising any or all of the IFNA2 variants or IFNA2-fusion proteins described herein. Kits of the invention include one or more containers comprising an IFNA2 variant or IFNA2 fusion protein described herein and instructions for use in accordance with any of the methods of the invention described herein. Generally, these instructions comprise a description of administration of the IFNA2 variant or IFNA2 fusion protein for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an IFNA2 variant or an IFNA2 fusion protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an IFNA2 variant or an IFNA2 fusion protein. The container may further comprise a second pharmaceutically active agent.

Kits may optionally provide additional components such as buffers and interpretive information. Normally, the kit comprises a container and a label or package insert(s) on or associated with the container.

### EXAMPLES

The invention is further described in the following examples, which do not limit the scope of the invention described in the claims.

### Example 1: Validating IFNA2/IFNAR2 Interaction Network Using AlphaSeq™

Prior to measuring binding affinities of IFNA2 variants against the IFNAR2 receptor, the wild-type IFNA2 and IFNAR2 interactions were validated using the AlphaSeq™ assay for PPI screening. Various construct designs for human, cynomolgus monkey, mouse, and rat IFNA2 and IFNAR2 were designed and constructed into AlphaSeq™ yeast strains. An AlphaSeq™ assay was performed to measure all the pairwise interactions between IFNA2 and IFNAR2 construct designs.

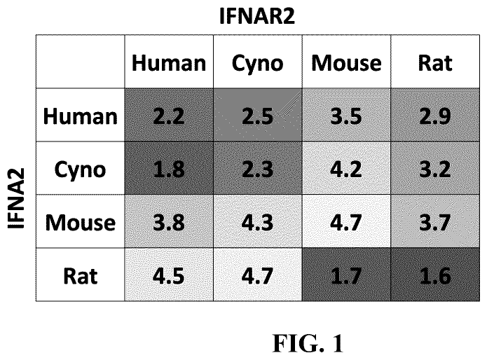

AlphaSeq™ predicted affinities (log 10 Kd nM) for the IFNA2/IFNAR2 validation network are shown in FIG. 1. Strong binding is considered below 3 (dark gray), weak binding is considered between 3 and 4 (medium gray), no detectable binding is considered above 4 (lightest gray).

Strong binding was observed between human IFNA2 to human, cynomolgus, and rat IFNAR2 (AlphaSeq™ Kd<3) and weak binding of human IFNA2 to mouse IFNAR2 (AlphaSeq™ Kd 3.5). For cynomolgus IFNA2 strong binding to human and cynomolgus IFNAR2 (AlphaSeq™ Kd<3), weak binding to rat IFNAR2 (AlphaSeq™ Kd 3.7) and no detectable binding to mouse IFNAR2 (AlphaSeq™ Kd 4.2) was observed. Weak binding of mouse IFNA2 was observed to human and mouse IFNAR2 (AlphaSeq™ Kd<4) and no detectable binding to cynomolgus or rat IFNAR2 (AlphaSeq™ Kd>4). Lastly, rat IFNA2b bound strongly to mouse and rat IFNAR2 (AlphaSeq™ Kd<3) and did not bind human or cynomolgus IFNAR2 (AlphaSeq™ Kd>4).

### Example 2: Measuring Binding Affinities of IFNA2 Variants Using AlphaSeq

To identify IFNA2 variants with decreased affinity for human IFNAR2 using AlphaSeq™, a site saturation mutagenesis (SSM) library of IFNA2 was constructed, such that each of the 161 amino acid residues in the displayed protein was mutated to every other amino acid, excluding cysteine. The variant library consisted of 2898 total variant IFNA2 proteins, which was combined with 161 copies of the wild-type IFNA2 sequence, such that the total library consisted of 3059 proteins. These 3059 proteins were synthesized and cloned into yeast display libraries and associated with nucleotide barcodes as described above. Each protein was displayed as a synthetic adhesion protein (SAP) fusion protein to the yeast agglutination factor AGA2 and the c-myc epitope tag.

Using AlphaSeq™, affinity predictions were obtained for interactions between each IFNA2 SSM variant and human, cynomolgus monkey (cyno), mouse, and rat IFNAR2. Variants of interest were those with decreased affinity (higher Kd values) than the wild-type IFNA2/IFNAR2 interaction. In the AlphaSeq™ assay, if a given variant has apparently decreased affinity, this could be due to true detuning—i.e., the affinity between IFNA2 and IFNAR2 is actually decreased—or from a decrease in expression or stability of the particular IFNA2 variant relative to wild-type. For therapeutic applications, the former is preferred. Thus, a parallel assay was performed to measure the surface expression of each displayed IFNA2 variant.

To measure surface expression, the library of variants was labeled with a FITC-conjugated anti-c-myc antibody. Labeled cells were then sorted by FACS into one of three bins—low (bin 1), medium (bin 2), or high (bin 3)—based on fluorescence intensity. Cells expressing higher levels of IFNA2 were expected to be enriched in the high bin and vice versa for those cells expressing lower levels of IFNA2. DNA was extracted from each of the three sorted samples and subjected to deep Illumina™ sequencing at the barcode locus. The number of reads aligning to each barcode was counted for each bin and normalized to the total number of reads in that bin and to the number of reads in the unsorted library, which results in an enrichment score for each barcode in each bin.

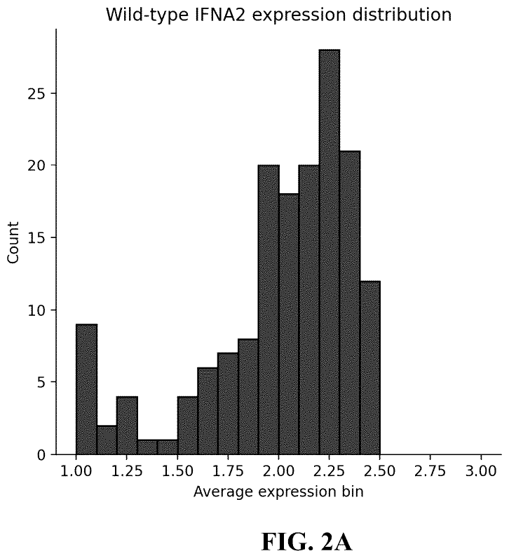

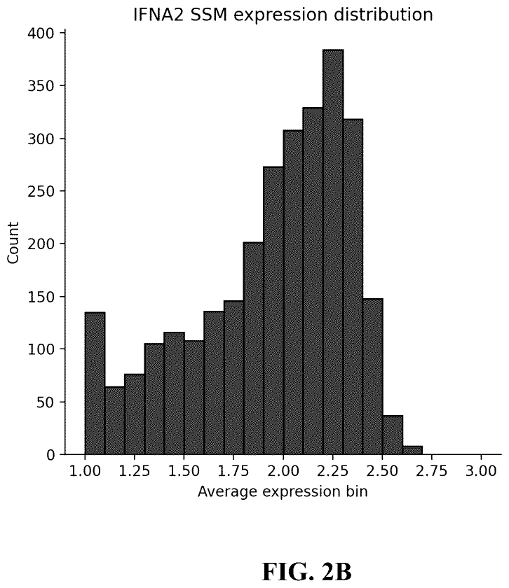

The enrichment scores for the 3 bins were combined into an expression value using the formula (1*bin1_enrichment+2*bin2_enrichment+3*bin3_enrichment)/(bin1_enrichment+bin2_enrichment+bin3_enrichment). This expression metric is termed the average expression bin for each barcode and returns a value between 1 and 3. Very highly expressing variants (i.e., those with most barcode reads in bin 3) have an average expression bin value approaching 3, while very lowly expressing variants (i.e., those with most reads in bin 1) have a value approaching 1. The average expression bin values for the various barcodes associated with each single IFNA2 variant were combined as a weighted average based on frequency in the unsorted population to obtain a value for each protein. The distribution of average expression bin values for the wild-type copies of IFNA2 are shown in FIG. 2A and the values for the members of the IFNA2 SSM library are shown in FIG. 2B.

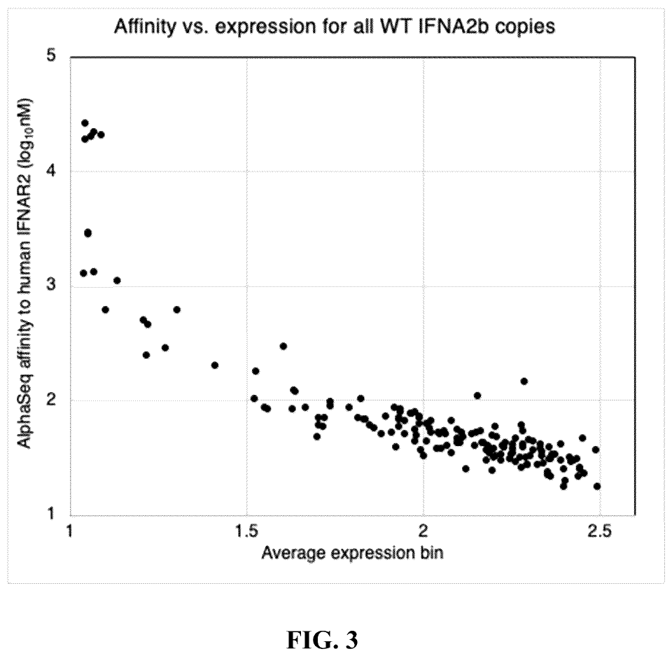

Using the 161 library-encoded copies of wild-type IFNA2, the relationship between expression and AlphaSeq™ affinity to human IFNAR2 was measured. As shown in FIG. 3, this analysis revealed a clear correlation where, as expected, poorly expressing proteins also show weaker binding. Based on this analysis, an average expression bin cutoff of 2.0 was set for selecting detuned IFNA2 variants, since below that expression level, weaker affinity was likely to be dependent on poor expression as opposed to bona fide detuning.

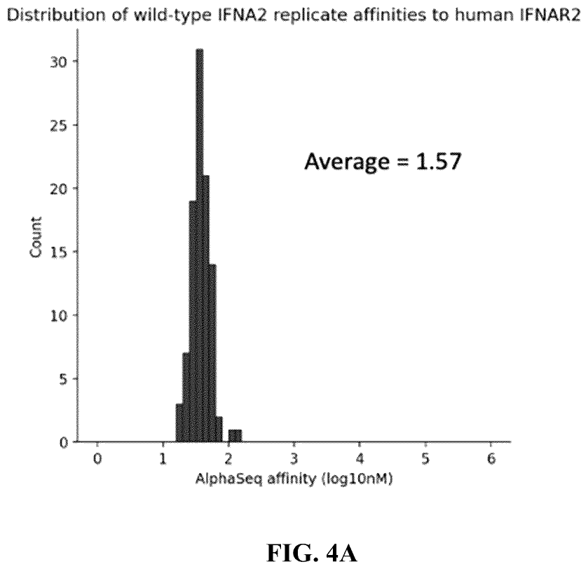

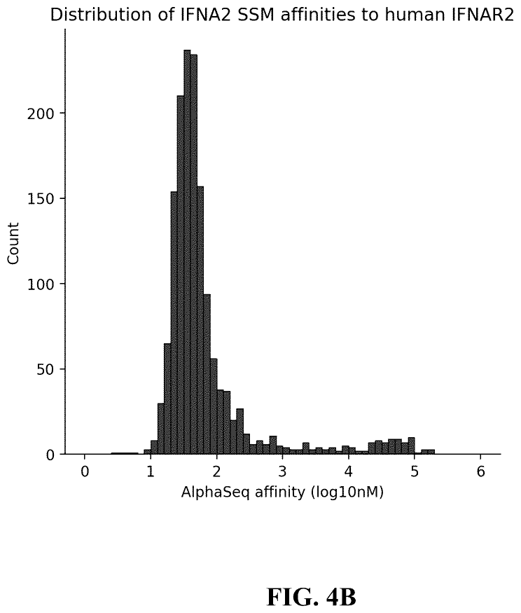

After filtering AlphaSeq™ results to include only variants with the average expression bin>2.0, the average affinity between the 161 wild-type IFNA2 replicates and human IFNAR2 was found to be 37 nM (log₁₀ nM=1.57 in FIG. 4A). While the 161 wild-type copies of IFNA2 had a tight affinity range centered at 37 nM, the IFNA2 SSM displayed a wide range of affinities to human IFNAR2: most variants bind with wild-type or near-wild-type affinity to IFNAR2, but a range of affinities was observed ranging from minimally detuned (˜100 nM Kd/log₁₀ nM=2) to background-level binding (˜100 μM Kd or weaker/log₁₀ nM=4 or greater) (FIG. 4B). Any variant found to bind human IFNAR2 with an affinity weaker than 100 nM (log₁₀ nM=2) was designated as “detuned.” These detuned IFNA2 variants are listed in Table 1, including affinity to human IFNAR2 (nM) and average expression bin.

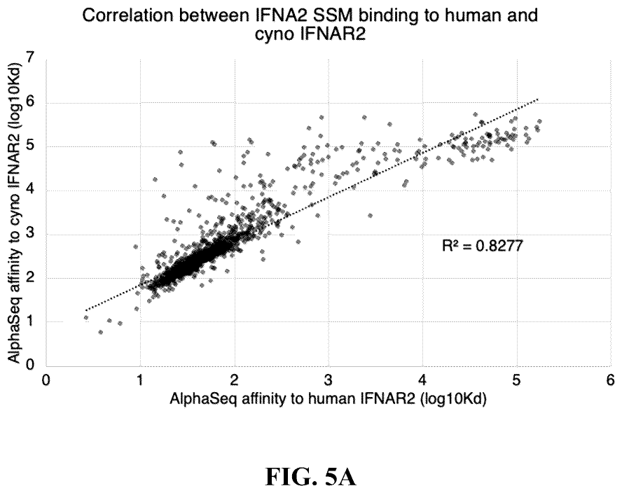

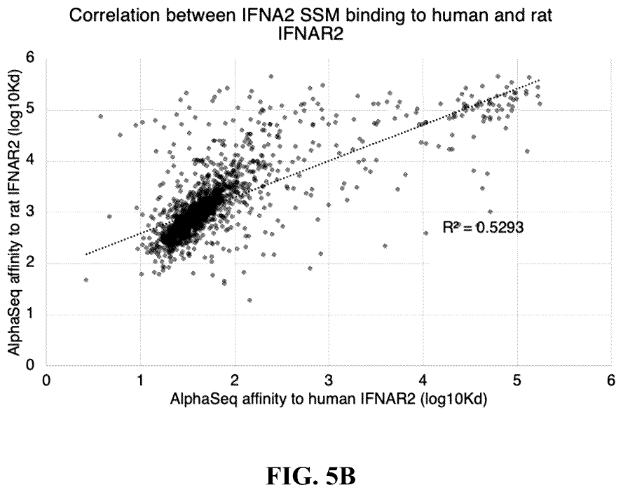

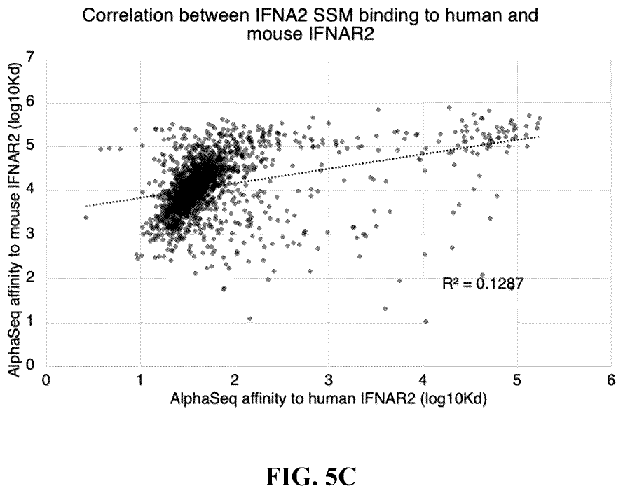

The AlphaSeq™ platform allowed simultaneous measurement of IFNA2 variant affinities to the cynomolgus, rat, and mouse orthologs of IFNAR2. IFNA2 variants showed highly correlated degrees of detuning to human and cynomolgus IFNAR2 (FIG. 5A). Detuning to rat IFNAR2 was moderately correlated with human (FIG. 5B). IFNA2 bound only weakly to mouse IFNAR2, which led to minimal correlation between human detuning and mouse detuning (FIG. 5C).

In addition to the above criteria for identifying all detuned variants, a more selective set of criteria were used for identifying and ranking high-confidence detuned variants for prioritization and further characterization. To qualify for prioritization and further characterization first, >50 diploids must have formed between the IFNA2 variant and human IFNAR2 during the AlphaSeq™ assay. In addition, >2 different substitutions at the specific position in IFNA2 must have given rise to detuning. For a given position, the number of substitutions that led to detuning was termed the “multiplicity” for that position. Preference was also given to variants at positions conserved in cynomolgus, mouse, and rat, as well as variants with similar degrees of detuning in cynomolgus and rat. Identifying variants with similar degrees of detuning in mouse was not possible given the weak interaction between human IFNA2 and mouse IFNAR2. Using these criteria, 12 initial variants were identified that spanned 4 affinity bins ranging from ˜300 nM (˜10-fold weaker than wild-type) to ˜10 μM (˜300-fold weaker than wild-type). These variants are described in Table 2.

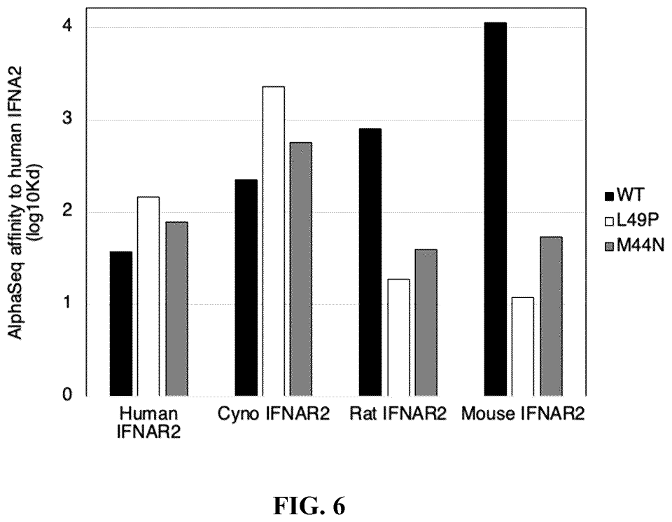

While wild-type IFNA2 bound only weakly to mouse IFNAR2 (FIG. 5C), two IFNA2 variants, L49P and M44N, were identified that bound to mouse IFNAR2 with substantially increased affinity, i.e., substantially decreased Kd values (FIG. 6). To identify IFNA2 variants with detuned affinities to both human and mouse IFNAR2, two further SSM sub-libraries were constructed in which either the L49P or M44N substitution was held constant. These libraries contained SSM mutations only in the second half of the IFNA2 protein, beginning with residue K106. These libraries were subjected to the same expression analysis and filtering as described for the wild-type library above, and IFNA2 variants were identified based on having an average expression bin>2.0 and demonstrating detuning against human IFNAR2, mouse IFNAR2 or both.

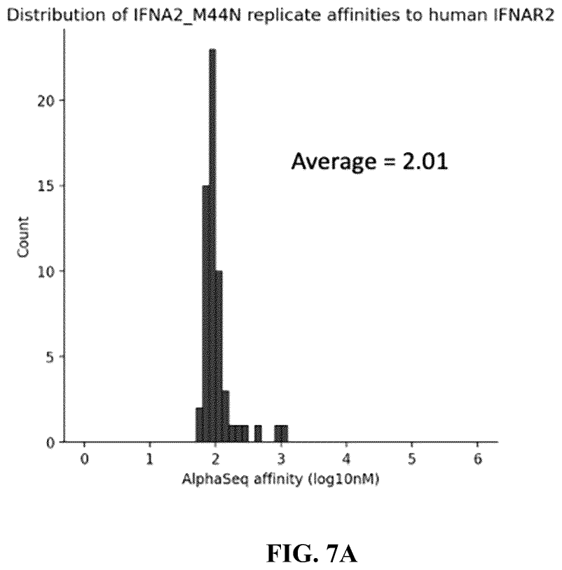

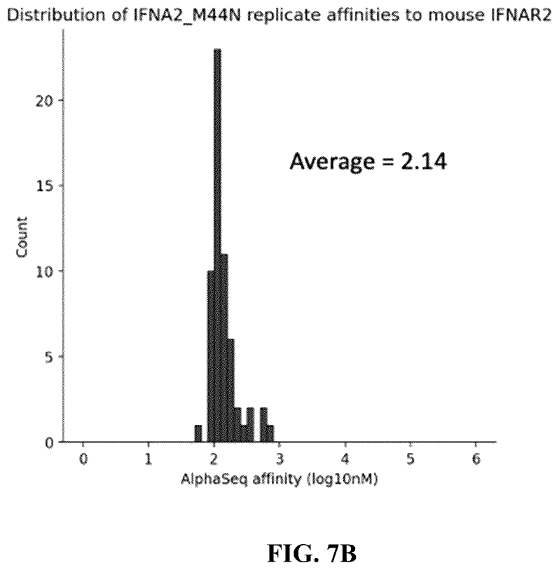

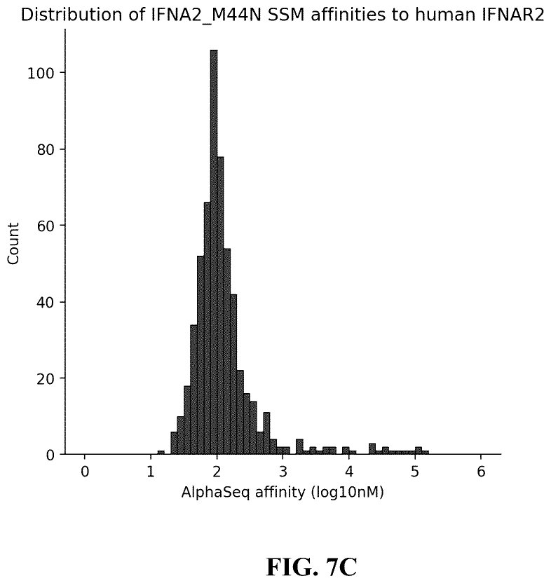

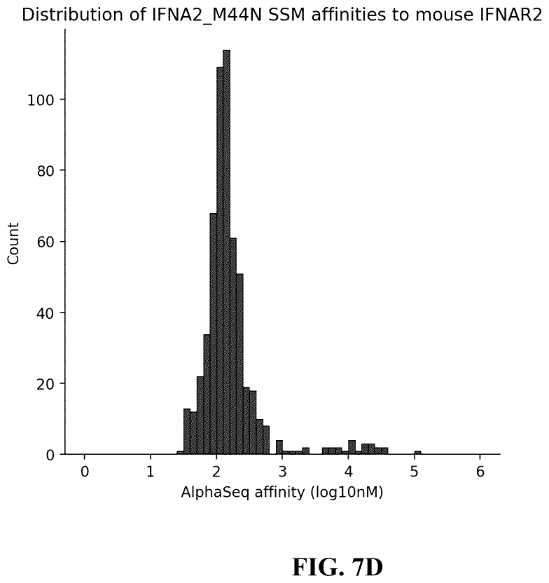

IFNA2_M44N bound to human IFNAR2 with an average replicate Kd of 102 nM (log₁₀ nM=2.01 in FIG. 7A) and to mouse IFNAR2 with an average replicate Kd of 138 nM (log₁₀ nM=2.14 in FIG. 7B). The M44N SSM library demonstrated a range of binding affinities against human and mouse receptors (FIG. 7C, FIG. 7D).

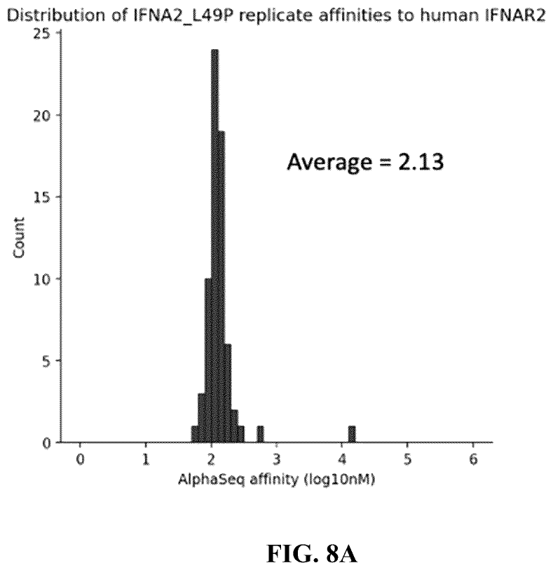

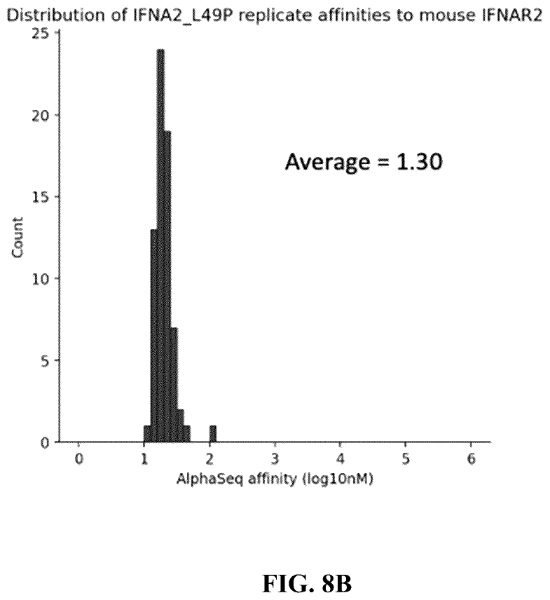

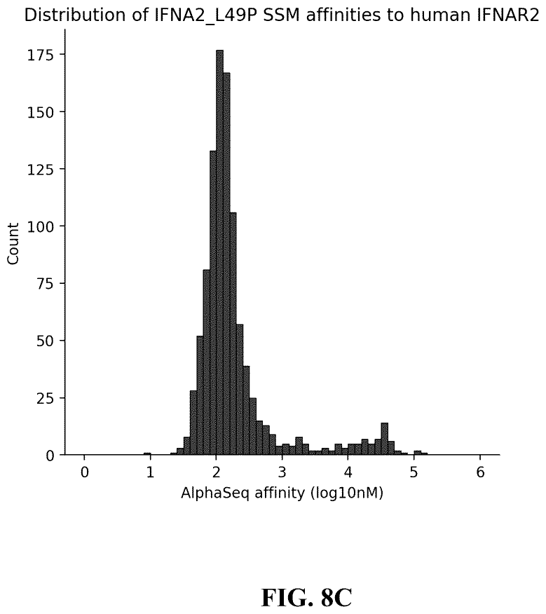

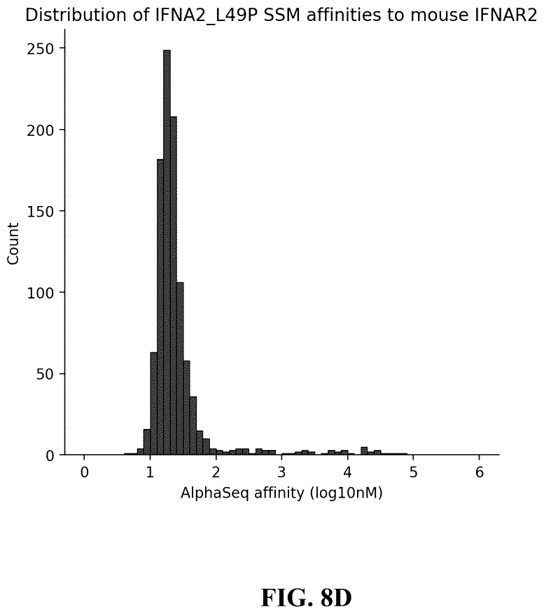

IFNA2_L49P bound to human IFNAR2 with an affinity of 135 nM (log₁₀ nM=2.13 in FIG. 8A) and to mouse IFNAR2 with an affinity of 20 nM (log₁₀ nM=1.30 in FIG. 8B). The L49P SSM library demonstrated a range of binding affinities against human and mouse receptors (FIG. 8C, FIG. 8D).

For both the M44N and L49P SSM libraries, any variant binding to human IFNAR2 with an affinity weaker than 316 nM (˜0.5-log weaker than the L49P and M44N single mutants bind to human IFNAR2) was deemed a detuned variant. Because L49P and M44N bound mouse IFNAR2 with significantly different affinities, a different affinity cutoff was used for each sub-library in determining detuned variants: 407 nM in the case of M44N and 63 nM in the case of L49P (˜0.5-log weaker than IFNA2_M44N and IFNA2_L49P bound mouse IFNAR2 respectively). From the M44N and L49P libraries, 127 variants were identified with detuned affinity against human IFNAR2 (Table 3), 22 variants were identified with detuned affinity against mouse IFNAR2 (Table 4), and 100 variants were identified with detuned affinity against both mouse and human IFNAR2 (Table 5).

### Example 3: Mammalian Protein Expression and Purification of Detuned IFNA2 Variant Fc Fusion Proteins

Cytokine variant sequences identified with appropriate AlphaSeq™ expression and affinity bins of interest were nominated for recombinant protein expression. Double stranded DNA fragments coding the identified variant sequence were synthesized by Twist Biosciences which included flanking sequences to facilitate cloning into the pCDNA3.1(+) mammalian cell expression vector. The commercial vector had been modified to encode the human IgG1 Fc sequence containing effectorless function mutations. Variant cytokine insert fragments were cloned into the modified expression vector using Gibson Assembly and transformed into host TOP10 E. coli competent cells. Cytokine sequence of interest was verified by Sanger sequencing. Confirmed sequence plasmid DNA was isolated from bacteria cultures using commercial DNA miniprep kits. The ExpiCHO mammalian cell line was thawed and cultured according to manufacturer instructions. Transient transfection of ExpiCHO cells was performed with detuned cytokine Fc construct DNA according to ExpiCHO transfection kit specifications and cultured supernatants were harvested 7 days later. Cultured supernatants containing proteins of interest were passed through a Protein A column using an AKTA FPLC instrument and eluted with low pH buffer. Purified protein was formulated in 20 mM histidine at pH 6.0 and quantitated using Nanodrop A280 measurements with yields of expression titers reported in Table 6. Table 6 also provides the polypeptide sequences of the IFNA2-Fc fusion proteins as SEQ ID NOs 539-550.

TABLE 6
Protein Expression Yields for IFNA2 Fc Fusion Proteins

| | Expression | | SEQ |
|---|---|---|---|
| | titers | | ID |
| Variant | (ug/ml) | Polypeptide Sequence | NO |
| IFNA2b_ | 45 | DIEPKSSDKTHTCPPCPAPEAAGGPSVFLFPPKPKDTLMIS | 539 |
| I47P | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRPSLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLRSKE | |
| IFNA2b_ | 72 | DIEPKSSDKTHTCPPCPAPEAAGGPSVFLFPPKPKDTLMIS | 540 |
| A168G | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRGEIMRSFSLSTNLQESLRSKE | |
| IFNA2b_ | 30 | DIEPKSSDKTHTCPPCPAPEAAGGPSVFLFPPKPKDTLMIS | 541 |
| R172T | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMTSFSLSTNLQESLRSKE | |
| IFNA2b_ | 16 | DIEPKSSDKTHTCPPCPAPEAAGGPSVFLFPPKPKDTLMIS | 542 |
| R46F | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRFISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLRSKE | |
| IFNA2b_ | 54 | DIEPKSSDKTHTCPPCPAPEAAGGPSVFLFPPKPKDTLMIS | 543 |
| S175K | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFKLSTNLQESLRSKE | |
| IFNA2b_ | 62 | DIEPKSSDKTHTCPPCPAPEAAGGPSVFLFPPKPKDTLMIS | 544 |
| M171L | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEILRSFSLSTNLQESLRSKE | |
| IFNA2b_ | 78 | DIEPKSSDKTHTCPPCPAPEAAGGPSVFLFPPKPKDTLMIS | 545 |
| S183W | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQEWLRSKE | |
| IFNA2b_ | 40 | DIEPKSSDKTHTCPPCPAPEAAGGPSVFLFPPKPKDTLMIS | 546 |
| N116W | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLWDLEACVIQGVGVTETPLMKEDSILAVRKYFQR | |
| | | ITLYLKEKKYSPCAWEVVRAEIMRSFSLSTNLQESLRSKE | |
| IFNA2b_ | 54 | DIEPKSSDKTHTCPPCPAPEAAGGPSVFLFPPKPKDTLMIS | 547 |
| F50G | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLGSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLRSKE | |
| IFNA2b_ | 4 | DIEPKSSDKTHTCPPCPAPEAAGGPSVFLFPPKPKDTLMIS | 548 |
| M82G | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEGIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLRSKE | |
| IFNA2b_ | 104 | DIEPKSSDKTHTCPPCPAPEAAGGPSVFLFPPKPKDTLMIS | 549 |
| F61G | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGGPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLRSKE | |
| IFNA2b_ | 51 | DIEPKSSDKTHTCPPCPAPEAAGGPSVFLFPPKPKDTLMIS | 550 |
| R185D | | RTPEVTCVVVDVSHEDPEVKFNWYVDGVEVHNAKTKPREEQ | |
| | | YNSTYRVVSVLTVLHQDWLNGKEYKCKVSNKALGAPIEKTI | |
| | | SKAKGQPREPQVYTLPPSRDELTKNQVSLTCLVKGFYPSDI | |
| | | AVEWESNGQPENNYKTTPPVLDSDGSFFLYSKLTVDKSRWQ | |
| | | QGNVFSCSVMHEALHNHYTQKSLSLSPGKGGGGSGGGGSCD | |
| | | LPQTHSLGSRRTLMLLAQMRRISLFSCLKDRHDFGFPQEEF | |
| | | GNQFQKAETIPVLHEMIQQIFNLFSTKDSSAAWDETLLDKF | |
| | | YTELYQQLNDLEACVIQGVGVTETPLMKEDSILAVRKYFQR | |
| | | ITLYLKEKKYSPCAWEVVRAEIMRSFSLSTNLQESLDSKE | |

### Example 4: Biolayer Interferometry Affinity Determination of Detuned IFNA2 Variant Fc Fusion Proteins

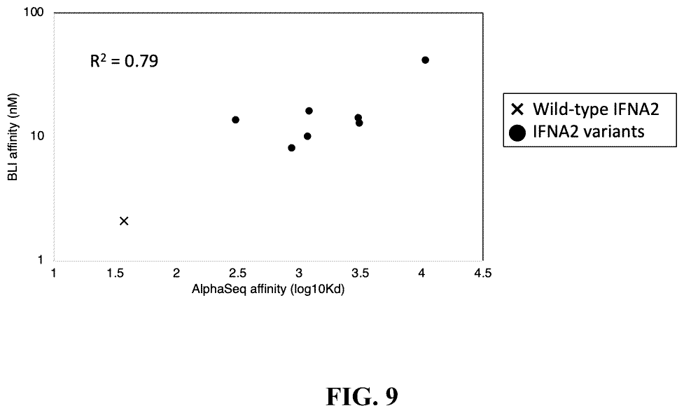

The binding kinetics for IFNA2 variant Fc fusion proteins was determined by bio-layer interferometry analysis on a Gator Prime instrument. This assay was performed by immobilizing commercially available recombinant human IFNalpha-beta R2, his tag (Acro Biosystems) to anti-His biosensors (Gator). Protein A purified IFNA2 variant Fc fusion proteins association to and dissociation from the immobilized IFNalpha-beta R2 was observed at the following dilution range (164 nM, 54 nM, 18 nM, 6 nM, 2 nM, 0.6 nM, and 0.2 nM) for all variants observed. Specifically, anti-his probes were hydrated in kinetics buffer (1x PBS with 0.2% BSA and 0.02% Tween-20) for 10 minutes and IFNalpha-beta R2, his tag antigen was immobilized to the anti-his probe for 180 seconds. Association was observed by placing probes with immobilized antigen into wells containing IFNA2 variant Fc fusion proteins for 60 seconds. Dissociation was measured after transferring the biosensors into wells containing only kinetics buffer for 60 seconds. All assay steps were performed with shaking at 1000 rpm at 30° C. Affinity constant (KD) analysis for this assay were determined using the Gator software provided from the manufacturer using data points collected from 60 seconds of the association step and the first 20 seconds of dissociation step, using a 1:1 global binding fit. Results of the kinetic studies are presented in Table 7. The bio-layer interferometry affinity measurements were highly correlated with AlphaSeq™ affinity measurements (FIG. 9).

TABLE 7
Affinity Determination of IFNA2 Fc Fusion Proteins

| Variant | KD affinity (nM) |
|---|---|
| IFNA2b_I47P | na |
| IFNA2b_A168G | 41.9 |
| IFNA2b_R172T | na |
| IFNA2b_R46F | na |
| IFNA2b_S175K | 12.9 |
| IFNA2b_M171L | 14.3 |
| IFNA2b_S183W | 16.2 |
| IFNA2b_N116W | 10.1 |
| IFNA2b_F50G | 8.13 |
| IFNA2b_M82G | na |
| IFNA2b_F61G | 13.7 |
| IFNA2b_R185D | na |
| IFNA2b_WT | 2.11 |

### Example 5: pSTAT1 T Cell Signaling Potency Determination of Detuned IFNA2 Variant Fc Fusion Proteins

Successfully expressed IFNA2 Fc fusion variant signaling potency in primary human PBMCs was determined using a phosphorylated STAT1 flow cytometry assay (pSTAT1 phosflow). Previously frozen human PBMCs (BloodworksBio) were thawed and counted to determine cell number and viability. Cell concentration was adjusted to 2 million viable human PBMC/ml and 100 μl was plated in round-well bottom 96 well tissue culture plates for a total of 200,000 cells per well. A 2X working stock of wild-type and variant IFNA2 Fc fusion proteins was prepared at a concentration of 2 μM before performing an five point serial dilution using 1:10 dilutions. 100 μl of the 2X titrated cytokines were added to 100 μl of human PBMC and the cells were cultured for 30 minutes. Cytokine signaling was stopped by the addition of 50 μl of 4% paraformaldehyde for an additional 30 minutes.

Cells were centrifuged and washed with PBS/1% BSA followed by resuspension in ice cold methanol to permeabilize cells. After 30 minutes, cells were washed twice in PBS/1% BSA before resuspending in 50 μl PBS/1% BSA containing human Fc block (ThermoFisher) at recommended concentration. Cells were incubated on ice for 20 minutes and staining antibody panel was prepared.

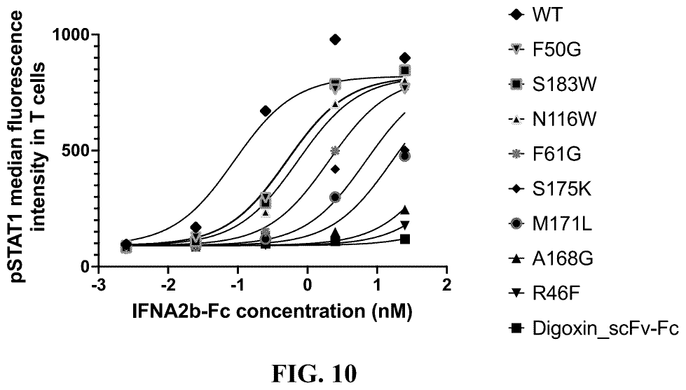

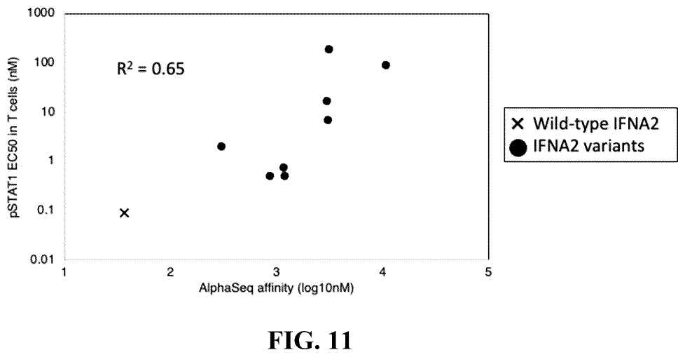

Anti-human CD14-BV421, anti-human CD20-PE, anti-human CD3-PECy5, and anti-human CD4-e450 were added to cells at recommended concentrations in an addition 50 μl volume. Cells were stained with antibodies for 30 minutes before washing twice with PBS/1% BSA and analyzing on a ZE5 (Biorad) flow cytometer. Data was analyzed using Flowjo software and EC50 potency curves determined using Graphpad Prism and reported in Table 8 and FIG. 10. The potency determined by STAT1 phosphorylation flow cytometry showed a strong correlation with affinity measurements between IFNA2 variants and IFNAR2 in AlphaSeq™. (FIG. 11)

TABLE 8
Signaling Potency Determination of IFNA2
Fc Fusion Proteins in Primary Human PBMCs

| Variant | EC50 in T cells (nM) |
|---|---|
| IFNA2b_WT | 0.08891 |
| IFNA2b_A168G | 88.01 |
| IFNA2b_R46F | 185.8 |
| IFNA2b_S175K | 6.923 |
| IFNA2b_M171L | 16.5 |
| IFNA2b_S183W | 0.5114 |
| IFNA2b_N116W | 0.7271 |
| IFNA2b_F50G | 0.5013 |
| IFNA2b_F61G | 2.035 |

Table 8 shows EC50 values for a subset of IFNA2-Fc fusion proteins as measured by phosphorylation of STATI in a phospho-specific flow cytometry assay. EC50 was measured in T cells (CD3+). IFNA2 variants showed a range of EC50 values, with the weakest variant (R46F) being ˜2000-fold less potent than wild-type (185.8 nM vs. 0.0889 nM).

### Example 6: IFNA2 Detuned Variant Candidate Immunocytokine Generation and Characterization

Lead IFNA2 variants displaying a range of correlated affinity and potency are selected for antibody fusion to generate immunocytokine candidates by cloning into modified pCDNA3.4 vector encoding scFv sequence that binds to CD8 and is fused to the same effectorless Fc described previously. IFNA2 immunocytokines are produced in ExpiCHO and are purified as described in Example 3. IFNA2 immunocytokine affinity and potency are determined as shown in Example 4 and Example 5, respectively. Detuned IFNA2 immunocytokine candidates that demonstrate cell specific signaling in CD8 cells with greatly reduced signaling in CD4 and other immune subsets are selected for further in vivo characterization.

### Example 7: Mouse Syngeneic Tumor Modeling Efficacy Study with IFNA2 Immunocytokines

Lead immunocytokine candidates are produced in ExpiCHO cells as before and purified proteins formulated in physiological buffer. Balb/c mice are implanted with 1 million CT26 colorectal carcinoma cells subcutaneously in the flank of the mice and tumor volume growth is measured with hand calipers. Dosing with IFNA2 detuned immunoctyokine commences when tumor volume reaches 100 mm3. 1 ug/kg immunocytokine is administered intravenously weekly for 3 weeks and tumor volumes are measured every three days. Mice are euthanized when tumor volumes exceed 2000 mm3. Percent tumor growth inhibition is calculated by dividing experimental tumor volumes by the average tumor volume of saline treated mice and multiplying by 100.

### Example 8: Detuned IFNA2 Immunocytokines Exhibit Increased Potency on CD8 T Cells

Lead IFNA2 variants displaying a range of correlated affinity as measured by AlphaSeq™ and potency were selected for antibody fusion to generate immunocytokine candidates by cloning into a modified pCDNA3.4 vector encoding scFv sequence that binds to CD8 and is fused to a human IgG1 effectorless Fc region. The expression vector therefore encoded IFNA2-anti-CD8 fusion proteins comprising an anti-CD8 antibody and each lead IFNA2 variant. Immunocytokines were produced in ExpiCHO cells and purified as described above in Example 3. Affinity constants (KD) for IFNA2-anti-CD8 (IFNA2-aCD8) fusion proteins binding to IFNalpha-beta R2 (IFNAR2) were determined using BLI as described above in Example 4. Results of the kinetic studies are presented in Table 9. As was the case for IFNA2-Fc fusion proteins, the variant IFNA2 proteins in the context of IFNA2-anti-CD8 fusion proteins demonstrated a range of affinities to IFNAR2. Table 9 also provides the polypeptide sequences of the IFNA2-aCD8 fusion proteins as SEQ ID NOs 551-559.

TABLE 9
Affinity Determination of IFNA2-aCD8 Fusion proteins

| | K_{D} | | SEQ |
|---|---|---|---|
| | affinity | | ID |
| Variant | (nM) | Polypeptide Sequence | NO |
| IFNA2b_ | K_{off} below | EVQLVESGGGLVQPGGSLRLSCAASGFNIKDTYIHFVRQAP | 551 |
| WT | limit of | GKGLEWIGRIDPANDNTLYASKFQGKATISADTSKNTAYLQ | |
| | detection | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQESLRSKE | |
| IFNA2b_ | 5.16 | EVQLVESGGGLVQPGGSLRLSCAASGFNIKDTYIHFVRQAP | 552 |
| I47P | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKENWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVESCS | |
| | | VMHEALHNHYTQKSLSLSPGKGGGGSGGGGSCDLPQTHSLG | |
| | | SRRTLMLLAQMRRPSLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQESLRSKE | |
| IFNA2b_ | 4.56 | EVQLVESGGGLVQPGGSLRLSCAASGFNIKDTYIHFVRQAP | 553 |
| R172T | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMTSFSLSTNLQESLRSKE | |
| IFNA2b_ | 0.93 | EVQLVESGGGLVQPGGSLRLSCAASGFNIKDTYIHFVRQAP | 554 |
| S175K | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRESGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFKLSTNLQESLRSKE | |
| IFNA2b_ | 5.93 | EVQLVESGGGLVQPGGSLRLSCAASGFNIKDTYIHFVRQAP | 555 |
| M171L | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKENWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEILRSFSLSTNLQESLRSKE | |
| IFNA2b_ | K_{off} below | EVQLVESGGGLVQPGGSLRLSCAASGFNIKDTYIHFVRQAP | 556 |
| S183W | limit of | GKGLEWIGRIDPANDNTLYASKFQGKATISADTSKNTAYLQ | |
| | detection | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQEWLRSKE | |
| IFNA2b_ | 0.40 | EVQLVESGGGLVQPGGSLRLSCAASGFNIKDTYIHFVRQAP | 557 |
| F50G | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVESCS | |
| | | VMHEALHNHYTQKSLSLSPGKGGGGSGGGGSCDLPQTHSLG | |
| | | SRRTLMLLAQMRRISLGSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQESLRSKE | |
| IFNA2b_ | 1.96 | EVQLVESGGGLVQPGGSLRLSCAASGFNIKDTYIHFVRQAP | 558 |
| F61G | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKFNWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGGPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQESLRSKE | |
| IFNA2b_ | 0.50 | EVQLVESGGGLVQPGGSLRLSCAASGENIKDTYIHFVRQAP | 559 |
| R185D | | GKGLEWIGRIDPANDNTLYASKFQGKATISADTSKNTAYLQ | |
| | | MNSLRAEDTAVYYCGRGYGYYVFDHWGQGTLVTVSSGGGGS | |
| | | GGGGSGGGGSDVQITQSPSSLSASVGDRVTITCRTSRSISQ | |
| | | YLAWYQQKPGKVPKLLIYSGSTLQSGVPSRFSGSGSGTDFT | |
| | | LTISSLQPEDVATYYCQQHNENPLTFGGGTKVEIKEPKSSD | |
| | | KTHTCPPCPAPEAAGGPSVFLFPPKPKDTLMISRTPEVTCV | |
| | | VVDVSHEDPEVKENWYVDGVEVHNAKTKPREEQYNSTYRVV | |
| | | SVLTVLHQDWLNGKEYKCKVSNKALGAPIEKTISKAKGQPR | |
| | | EPQVYTLPPSRDELTKNQVSLTCLVKGFYPSDIAVEWESNG | |
| | | QPENNYKTTPPVLDSDGSFFLYSKLTVDKSRWQQGNVFSCS | |
| | | VMHEALHNHYTQKSLSLSPGKGGGGSGGGGSCDLPQTHSLG | |
| | | SRRTLMLLAQMRRISLFSCLKDRHDFGFPQEEFGNQFQKAE | |
| | | TIPVLHEMIQQIFNLFSTKDSSAAWDETLLDKFYTELYQQL | |
| | | NDLEACVIQGVGVTETPLMKEDSILAVRKYFQRITLYLKEK | |
| | | KYSPCAWEVVRAEIMRSFSLSTNLQESLDSKE | |

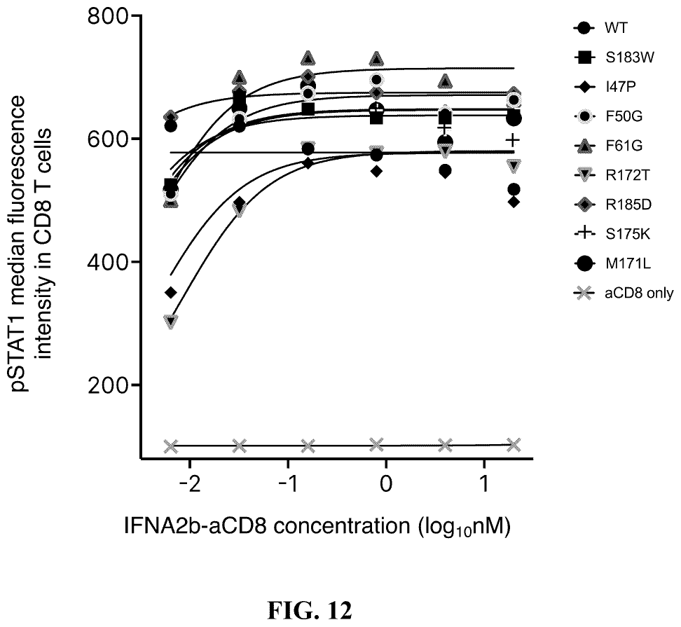

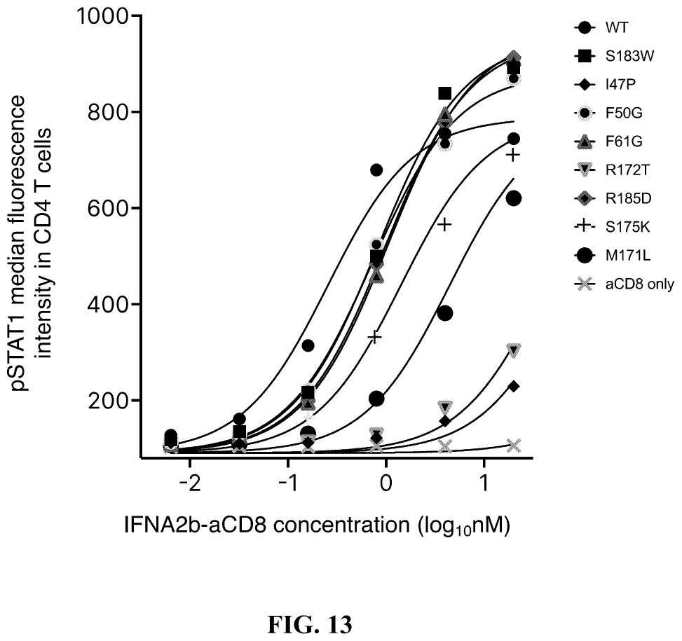

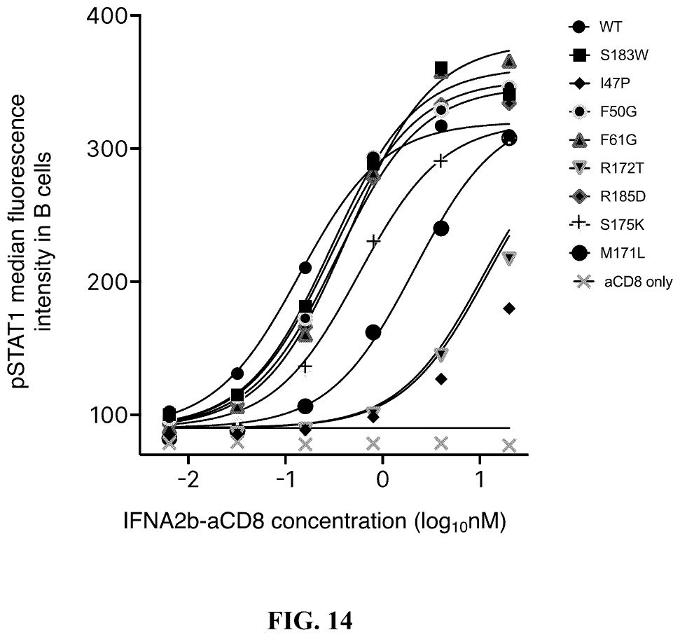

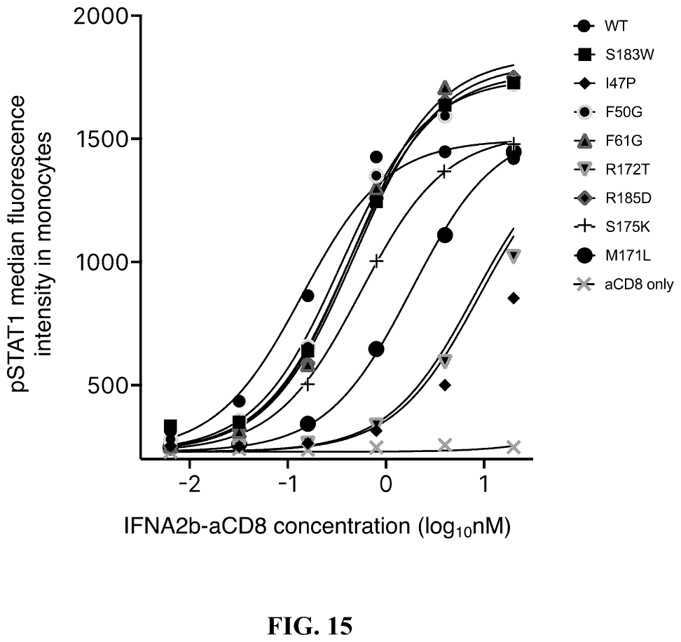

Cell-type-specific signaling potency was identified for each IFNA2-aCD8 fusion using the same PBMC pSTAT1 assay described in Example 5. Signaling was measured in the targeted CD8+ T cell population, as well as for three non-targeted cell populations (CD4+ T cells, B cells, and monocytes). Dose-response curves for STAT1 phosphorylation are shown in CD8 T cells (FIG. 12), CD4 T cells (FIG. 13), B cells (FIG. 14), and monocytes (FIG. 15). EC50 values are shown in Table 10. The concentrations of protein added in this assay ranged from 6.4 pM to 20 nM and those values are set as the minimum and maximum measurable EC50s, respectively, in Table 10. IFNA2-aCD8 fusions all showed higher potency in the targeted CD8 T cells than the non-targeted cell populations. For example, IFNA2_R172T had an EC50 of ˜8 pM in CD8 cells, but ˜8 nM, ˜11 nM, and >20 nM respectively in monocytes, B cells, and CD4 cells, a >1000-fold potency difference in targeted vs. non-targeted cells. These results indicate that detuned IFNA2-anti-CD8 immunocytokines comprising the IFNA2 variants disclosed herein mediated cis-dependent signaling in CD8 cells, with reduced affinity to IFNAR2 relative to wild-type IFNA2.

TABLE 10
Signaling Potency Determination of IFNA2-aCD8
Fusion Proteins in Primary Human PBMCs

| | EC50 in | EC50 in | EC50 in | EC50 in |
|---|---|---|---|---|
| | CD8 cells | CD4 cells | B cells | monocytes |
| | (nM) | (nM) | (nM) | (nM) |
| WT | <0.0064 | 0.2555 | 0.1388 | 0.1392 |
| S183W | <0.0064 | 0.8285 | 0.2942 | 0.4177 |
| I47P | <0.0064 | >20 | 11.88 | 8.987 |
| F50G | <0.0064 | 0.7211 | 0.298 | 0.3474 |
| F61G | <0.0064 | 1.06 | 0.4401 | 0.4533 |
| R172T | 0.008421 | >20 | 10.93 | 7.952 |
| R185D | <0.0064 | 0.9832 | 0.3375 | 0.4687 |
| S175K | <0.0064 | 1.417 | 0.5614 | 0.5493 |
| M171L | <0.0064 | 4.458 | 2.109 | 1.825 |

### Example 9: Selection of Further Detuned IFNA2 Variants

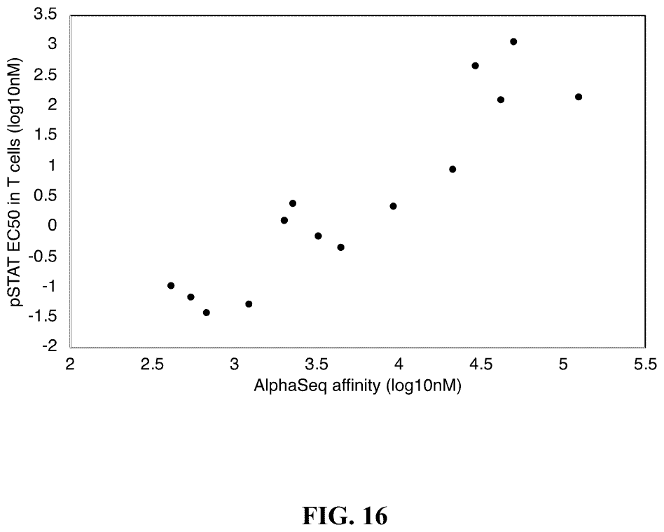

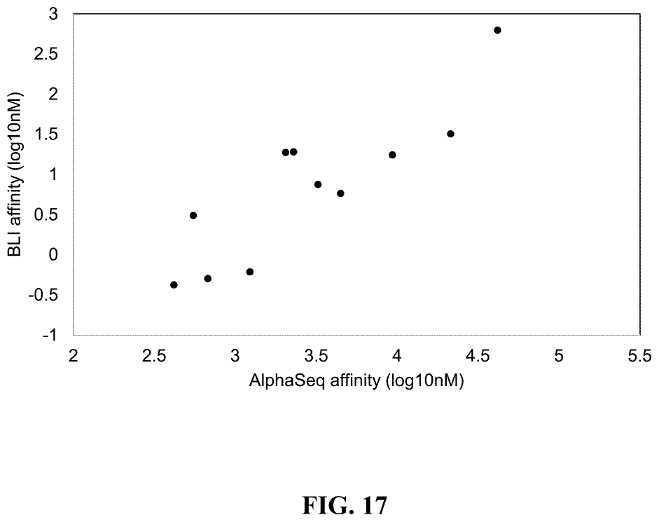

After characterizing the first batch of detuned IFNA2 variants described in Table 2, a further set of 15 IFNA2 variants was selected based on criteria described in Example 2 for selecting top detuned variants, but with a wider range of affinities, ranging from ˜400 nM to >100 M (affinities shown in Table 11). These proteins were produced as described in Example 8 as Fc-fusion proteins and tested for binding to IFNAR2 by BLI and for signaling in a human PBMC Phosflow assay using the same protocol as described in Example 8. Affinities of IFNA2 Fc fusion proteins to IFNAR2 as measured by BLI are shown in Table 12, with variants showing binding weaker than the limit of detection marked as “na.” Signaling potencies in human T cells are shown in Table 13. As in Examples 4 and 5, there was a strong correlation between AlphaSeq™ predicted affinity for IFNA2 variants and both BLI-measured affinity and Phosflow-measured signaling potency. FIG. 16 is a scatter plot showing the relation between the AlphaSeq™ affinity values and BLI-measured Kd for a second set of IFNA2 Fc fusion proteins comprising IFNA2 variants. FIG. 17 is a scatter plot showing the correlation between the AlphaSeq™ affinity values and Phosflow-measured pSTAT1 EC50 in T cells for a second set of IFNA2 Fc fusion proteins comprising IFNA2 variants. Table 11 also provides the polypeptide sequences of the IFNA2-Fc fusion proteins as SEQ ID NOs 560-574.

TABLE 11
Detuned IFNA2 variants produced as soluble Fc fusions

| | | | Position | | | |
|---|---|---|---|---|---|---|
| | | | conserved | | | |
| Affinity | | IFNAR2 | in | | | SEQ |
| bin | | affinity | mouse/cyno/ | | Polypeptide | ID |
| (log₁₀nM) | Variant | (log₁₀nM) | rat? | Multiplicity | Sequence | NO |
| >4.5 | A168R | 5.10 | yes | 9 | DIEPKSSDKTHTCPPCP | 560 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRREI | |
| | | | | | MRSFSLSTNLQESLRSK | |
| | | | | | E | |
| | R172D | 4.70 | yes | 12 | DIEPKSSDKTHTCPPCP | 561 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MDSFSLSTNLQESLRSK | |
| | | | | | E | |
| | R172N | 4.62 | yes | 12 | DIEPKSSDKTHTCPPCP | 562 |
| | | | | | APEAAGGPSVFLFPPKP | |
| | | | | | KDTLMISRTPEVTCVVV | |
| | | | | | DVSHEDPEVKENWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MNSFSLSTNLQESLRSK | |
| | | | | | E | |
| 4-4.5 | R56L | 4.47 | yes | 9 | DIEPKSSDKTHTCPPCP | 563 |
| | | | | | APEAAGGPSVFLFPPKP | |
| | | | | | KDTLMISRTPEVTCVVV | |
| | | | | | DVSHEDPEVKENWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDLHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSK | |
| | | | | | E | |
| | R167E | 4.35 | yes | 10 | DIEPKSSDKTHTCPPCP | 564 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVEAEI | |
| | | | | | MRSFSLSTNLQESLRSK | |
| | | | | | E | |
| | M171Y | 4.33 | cyno only | 14 | DIEPKSSDKTHTCPPCP | 565 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | YRSFSLSTNLQESLRSK | |
| | | | | | E | |
| 3.5-4 | R46D | 3.97 | yes | 11 | DIEPKSSDKTHTCPPCP | 566 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRDISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSK | |
| | | | | | E | |
| | S173H | 3.65 | cyno only | 7 | DIEPKSSDKTHTCPPCP | 567 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRHFSLSTNLQESLRSK | |
| | | | | | E | |
| | I47D | 3.51 | cyno only | 4 | DIEPKSSDKTHTCPPCP | 568 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRDSLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSK | |
| | | | | | E | |
| 3-3.5 | A168F | 3.36 | yes | 9 | DIEPKSSDKTHTCPPCP | 569 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRFEI | |
| | | | | | MRSFSLSTNLQESLRSK | |
| | | | | | E | |
| | V165N | 3.31 | yes | 14 | DIEPKSSDKTHTCPPCP | 570 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWENVRAEI | |
| | | | | | MRSFSLSTNLQESLRSK | |
| | | | | | E | |
| | S34A | 3.09 | no | 1 | DIEPKSSDKTHTCPPCP | 571 |
| | | | | | APEAAGGPSVFLFPPKP | |
| | | | | | KDTLMISRTPEVTCVVV | |
| | | | | | DVSHEDPEVKENWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGA | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSK | |
| | | | | | E | |
| 2.5-3 | K187R | 2.83 | cyno only | 10 | DIEPKSSDKTHTCPPCP | 572 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSR | |
| | | | | | E | |
| | F50S | 2.74 | cyno only | 8 | DIEPKSSDKTHTCPPCP | 573 |
| | | | | | APEAAGGPSVFLFPPKP | |
| | | | | | KDTLMISRTPEVTCVVV | |
| | | | | | DVSHEDPEVKFNWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLSS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSPCAWEVVRAEI | |
| | | | | | MRSFSLSTNLQESLRSK | |
| | | | | | E | |
| | P160E | 2.62 | mouse/rat | 5 | DIEPKSSDKTHTCPPCP | 574 |
| | | | | | APEAAGGPSVFLFPPKP | |
| | | | | | KDTLMISRTPEVTCVVV | |
| | | | | | DVSHEDPEVKENWYVDG | |
| | | | | | VEVHNAKTKPREEQYNS | |
| | | | | | TYRVVSVLTVLHQDWLN | |
| | | | | | GKEYKCKVSNKALGAPI | |
| | | | | | EKTISKAKGQPREPQVY | |
| | | | | | TLPPSRDELTKNQVSLT | |
| | | | | | CLVKGFYPSDIAVEWES | |
| | | | | | NGQPENNYKTTPPVLDS | |
| | | | | | DGSFFLYSKLTVDKSRW | |
| | | | | | QQGNVFSCSVMHEALHN | |
| | | | | | HYTQKSLSLSPGKGGGG | |
| | | | | | SGGGGSCDLPQTHSLGS | |
| | | | | | RRTLMLLAQMRRISLFS | |
| | | | | | CLKDRHDFGFPQEEFGN | |
| | | | | | QFQKAETIPVLHEMIQQ | |
| | | | | | IFNLFSTKDSSAAWDET | |
| | | | | | LLDKFYTELYQQLNDLE | |
| | | | | | ACVIQGVGVTETPLMKE | |
| | | | | | DSILAVRKYFQRITLYL | |
| | | | | | KEKKYSECAWEVVRAEI | |
| | | | | | MRSFSLSTNLQESLRSK | |
| | | | | | E | |

TABLE 12
Affinity Determination of IFNA2 Fc Fusion Proteins

| Variant | IFNAR2 KD (nM) |
|---|---|
| A168R | na |
| R172D | na |
| R172N | 625 |
| R56L | na |
| R167E | na |
| M171Y | 32.0 |
| R46D | 17.5 |
| S173H | 5.79 |
| I47D | 7.51 |
| A168F | 19.1 |
| V165N | 18.8 |
| S34A | 0.612 |
| K187R | 0.505 |
| F50S | 3.08 |
| P160E | 0.422 |

TABLE 13
Signaling Potency Determination of IFNA2
Fc Fusion Proteins in Primary Human PBMCs

| Variant | EC50 in T cells (nM) |
|---|---|
| A168R | 139.2 |
| R172D | 1181 |
| R172N | 124.2 |
| R56L | 459.5 |
| R167E | 8.978 |
| M171Y | 2.175 |
| R46D | 0.464 |
| S173H | 0.697 |
| I47D | 2.46 |
| A168F | 1.278 |
| V165N | 0.05168 |
| S34A | 0.03695 |
| K187R | 0.06664 |
| F50S | 0.1069 |
| P160E | 139.2 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated human interferon alpha-2 (IFNA2) variant, wherein the IFNA2 variant has decreased or no detectable binding to the human interferon-alpha/beta receptor beta 2 (IFNAR2) as compared to the wild-type human IFNA2 polypeptide, and wherein the IFNA2 variant comprises a polypeptide sequence of any one of SEQ ID NOS 10-416 and 439-538.

2. The isolated IFNA2 variant of claim 1, wherein the IFNA2 variant has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

3. An isolated nucleic acid encoding the IFNA2 variant of claim 1.

4. A recombinant expression vector comprising the nucleic acid of claim 3.

5. A host cell comprising the recombinant expression vector of claim 4.

6. The isolated IFNA2 variant of claim 1, wherein the IFNA2 variant comprises a polypeptide sequence of any one of SEQ ID NOs 67, 79, 85, 99-101, 117, 118, 121, 159, 161, and 165.

7. An isolated nucleic acid encoding the IFNA2 variant of claim 6.

8. An isolated fusion protein comprising:
an antibody or binding fragment thereof; and
an isolated human IFNA2 variant, wherein the IFNA2 variant is covalently linked to the antibody or binding fragment thereof, and wherein the IFNA2 variant has decreased or no detectable binding to the human interferon-alpha/beta receptor beta (IFNAR2) as compared to the wild-type human IFNA2 polypeptide, and wherein the IFNA2 variant comprises a polypeptide sequence of any one of SEQ ID NOs 10-416 and 439-538.

9. The isolated fusion protein of claim 8, wherein the IFNA2 variant has a binding affinity to the human interferon-alpha/beta receptor beta 2 (IFNAR2) that is decreased by 1.5-fold, 2-fold, 3-fold, 5-fold, 10-fold, 50-fold, 100-fold, 500-fold, 1000-fold, 2000-fold, or 3000-fold or more compared to the binding affinity between the wild-type human IFNA2 polypeptide and the human IFNAR2.

10. The isolated fusion protein of claim 8, wherein the IFNA2 variant is covalently linked to the antibody by a polypeptide linker.

11. The isolated fusion protein of claim 9, wherein the polypeptide linker is selected from the group consisting of (G4S)2 and (G4S)3.

12. The isolated fusion protein of claim 10, wherein the polypeptide linker is a polypeptide tag selected from the group consisting of FLAG, MYC, HA, and 6×his.

13. An isolated cell line that produces the fusion protein of claim 9.

14. An isolated nucleic acid encoding the fusion protein of claim 9.

15. A recombinant expression vector comprising the nucleic acid of claim 14.

16. A host cell comprising the recombinant expression vector of claim 15.

17. The isolated fusion protein of claim 9, wherein the antibody or binding fragment thereof comprises an Fc domain.

18. The isolated fusion protein of claim 10, wherein the isolated fusion protein comprises a polypeptide sequence of any one of SEQ ID NOs 539-550 and 560-574.

19. The isolated fusion protein of claim 9, wherein the antibody is an anti-CD8 antibody.

20. The isolated fusion protein of claim 10, wherein the isolated fusion protein comprises a polypeptide sequence of any one of SEQ ID NOs 552-559.
